(19) United States
(12) Patent Application Publication
(10) Pub. No.: US 2019/0233824 A1
(43) Pub. Date: Aug. 1, 2019

(54) PD-1 SPECIFIC APTAMERS

(71) Applicant: THE METHODIST HOSPITAL SYSTEM, Houston, TX (US)

(72) Inventors: Santhana Gowri THANGAVELU DEVARAJ, Houston, TX (US); Swaminathan P. IYER, Houston, TX (US)

(21) Appl. No.: 16/330,813

(22) PCT Filed: Sep. 6, 2017
(86) PCT No.: PCT/US17/50260

§ 371 Date: Mar. 6, 2019

Related U.S. Application Data
(60) Provisional application No. 62/383,952, filed on Sep. 6, 2016.

(51) Int. Cl. C12N 15/115; A61P 35/02; A61K 9/50; C07K 16/18; A61K 47/54; A61K 47/69

(52) U.S. Cl. CPC C12N 15/115; A61P 35/02; A61K 47/6925; C07K 16/18; A61K 47/549; A61K 9/50

(57) ABSTRACT

The present disclosure relates to PD1-specific aptamers and methods of treating cancer.

### CROSS REFERENCE TO RELATED APPLICATIONS

This application claims the benefit of U.S. Provisional Patent Application Ser. No. 62/383,952 filed Sep. 6, 2016, the disclosure of which is expressly incorporated herein by reference.

### FIELD

The present disclosure relates to PD1-specific aptamers and methods of treating cancer.

### BACKGROUND

A majority of human tumors have significant genetic and epigenetic changes that lead to the expression of tumor-associated antigens that trigger cytotoxic T-cell response, a favorable step for clearance of tumor cells. However, many check point mechanisms are in place to prevent autoimmunity which turns out to be beneficial for cancer cell proliferation. Cancer cells have been shown to secrete PD-L1 and PD-L2 and utilize PD-1 mediated inhibition of T-cell function for escape from immune surveillance leading to cancer progression (Pardoll, D M (2012) Nature Reviews 12: 252-264). Recently, several therapeutic antibodies have been developed that target PD-1/PD-L1 axis to activate T-cell response against tumors in mice and humans and have been FDA approved. However, therapeutic antibodies are not suitable for all the patients because of other immunological side effects.

### SUMMARY

Disclosed herein are DNA aptamers that mimic a therapeutic antibody that targets the PD-1/PD-L1 axis. High affinity DNA aptamers that selectively bind to important proteins involved in cancer progression are emerging as a new class of drugs and diagnostic agents. DNA aptamers are alternatives to small-molecule and antibody-based drugs due to their high specificity to therapeutic protein targets and their amenability for easy modification to carry a toxic cargo to kill cancer cells in the body.

In some aspects, disclosed herein is a single-stranded DNA aptamer, comprising the formula

n-L-n-Z-n-R-n,

wherein “L” comprises the nucleic acid sequence TACCTGATAGCGTATGCGA (SEQ ID NO:2), or a nucleic acid sequence having at least 80% sequence identity to SEQ ID NO:2, or a fragment thereof at least 15 nucleotides in length,

wherein “Z” comprises the nucleic acid sequence GGGxTTGGTGTGGTGGG (SEQ ID NO:1), wherein “x” is A, T, or C,

wherein “R” comprises the nucleic acid sequence CTCTCAGTAGGTGCATAAGCG (SEQ ID NO:3), or a nucleic acid sequence having at least 80% sequence identity to SEQ ID NO:3, or a fragment thereof at least 15 nucleotides in length, and

wherein each “n” is independently any 0 to 10 nucleotides,

wherein the DNA aptamer specifically binds to PD-1 and disrupts the interaction between Programmed Death-1 (PD-1) and Programmed Death Ligand-1 (PD-L1) under physiological conditions.

In some embodiments, the DNA aptamer comprises a nucleic acid sequence selected from the group consisting of SEQ ID NO:6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, and 63.

In some embodiments, the DNA aptamer comprises a nucleic acid sequence having at least 80% sequence identity to a nucleic acid sequence selected from the group consisting of SEQ ID NO:6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, and 63.

Also disclosed is a therapeutic composition comprising a DNA aptamer disclosed herein in a pharmaceutically acceptable carrier. In some embodiments, the DNA aptamer is encapsulated in a nanoparticle. For example, the DNA aptamer can be PEGylated.

In some embodiments, the DNA aptamers described herein act as a modular portion of a compound to which additional modular units can be added. In some embodiments, the aptamer is conjugated to an additional aptamer. In some embodiments, the aptamer is conjugated to an antibody or antibody fragment. In some embodiments, the DNA aptamer conjugated to an additional aptamer acts as a bispecific aptamer. In some embodiments, the DNA aptamer conjugated to an antibody acts as a bispecific aptamer/antibody molecule. In some embodiments, the DNA aptamers described herein are conjugated with at least one (for example, at least one, at least two, at least three, at least four, at least five) additional aptamer(s). In some embodiments, the DNA aptamers described herein are conjugated with at least one (for example, at least one, at least two, at least three, at least four, at least five) antibody or antibody fragment.

In some embodiments, the aptamer is conjugated to a therapeutic agent, such as an antineoplastic agent and/or a radiosensitizer. In some embodiments, the aptamer is administered in combination with a therapeutic agent, such as an antineoplastic agent and/or a radiosensitizer.

Anti-neoplastic agents (antineoplastic drugs) include, but are not limited to, abarelix, abiraterone, ado-trastuzumab emtansine, afatinib, aflibercept, aldesleukin, alectinib, alemtuzumab, alitretinoin, altretamine, amsacrine, anagrelide, anastrozole, antithymocyte globulin, arsenic trioxide, asparaginase, atezolizumab, axitinib, azacitidine, BCG, belinostat, bendamustine, bevacizumab, bexarotene, bicalutamide, bleomycin, blinatumomab, bortezomib, bosutinib, brentuximab vedotin, busulfan, cabazitaxel, cabozantinib, capecitabine, carboplatin, carfilzomib, carmofur, carmustine, chlorambucil, chlormethine, ceritinib, cetuximab, chidamide, cisplatin, cladribine, clofarabine, cobimetinib, crizotinib, cyclophosphamide, cyproterone, cytarabine, dabrafenib, dacarbazine, dactinomycin, daratumumab, daunorubicin, dasatinib, decitabine, degarelix, denileukin diftitox, dinutuximab, docetaxel, doxorubicin, elotuzumab, enzalutamide, epirubicin, etoposide, eribulin, erlotinib, estramustine, everolimus, exemestane, floxuridine, fludarabine, fluoxymesterone, flutamide, fotemustine, fulvestrant, gefitinib, gemtuzumab ozogamicin, goserelin, histrelin, hydroxycarbamide, hydroxyurea, ibritumomab tiuxetan, ibrutinib, ibritumomab tiuxetan, idarubicin, idelalisib, ifosfamide, imatinib, imiquimod, interferon, iobenguane, ipilimumab, irinotecan, isotretinoin, leuprolide, lomustine, melphalan, methotrexate, mercaptopurine, mifepristone, mitomycin, mitotane, mitoxantrone, nab-paclitaxel, necitumumab, nedaplatin, nelarabine, nilotinib, nilutamide, nivolumab, obinutuzumab, octreotide, ofatumumab, olaparib, omacetaxine, osimertinib, oxaliplatin, palbociclib, paclitaxel, panitumumab, panobinostat, pazopanib, pembrolizumab, pemetrexed, pertuzumab, pentostatin, plicamycin, pomalidomide, ponatinib, pralatrexate, procarbazine, radium Ra 223 dichloride, raltitrexed, ramucirumab, regorafenib, rituximab, romidepsin, ruxolitinib, samarium (153Sm) lexidronam, siltuximab, sipuleucel-T, sonidegib, sorafenib, strontium-89, sunitinib, talimogene laherparepvec, tamibarotene, tamoxifen, tegafur, tegafur/uracil, temozolomide, temsirolimus, teniposide, thalidomide, tioguanine, topotecan, toremifene, tocilizumab, tositumomab, trabectedin, trametinib, trastuzumab, trastuzumab emtansin, tretinoin, trifluridine/tipiracil combination, triptorelin, valproate, valrubicin, vandetanib, vemurafenib, venetoclax, vinblastine, vincristine, vindesine, vinflunine, vismodegib, vorinostat, ziv-aflibercept. In some embodiments, the antineoplastic agent comprises hydroxyurea. In some embodiments, the antineoplastic agent comprises interferon. In some embodiments, the antineoplastic agent comprises venetoclax.

Examples of known radiosensitizers include gemcitabine, 5-fluorouracil, pentoxifylline, and vinorelbine.

In some embodiments, the aptamer is tethered DNA “boxcars” to form a “DNA nanotrain” for targeted transport of molecular drugs. DNA “boxcars” are tandem dsDNA sequences that are targets for chemotherapeutic drug intercalation. It is well known that several widely-used anthracycline anticancer drugs, including doxorubicin (Dox), daunorubicin (DNR), and epirubicin (EPR), can preferentially intercalate into double-stranded 5′-GC-3′ or 5′-CG-3′. Therefore, the boxcars can contain drug intercalation sites, such as ACG/CGT.

Also disclosed are polyvalent aptamers comprising two or more of the disclosed aptamers that mimic therapeutic antibody that target PD-1/PD-L1 axis.

In some embodiments, the DNA aptamer is present at a concentration from 10 nM to 200 μM.

In some embodiments, the methods disclosed herein are used to treat a cancer in a subject, for example, a hematologic malignancy. In some embodiments, the hematologic malignancy includes, but is not limited to, leukemia, lymphoma, myeloid disorder, lymphoid disorder, myelodysplastic syndrome (MDS), myeloproliferative disease (MPD), mast cell disorder, and myelonia (e.g., multiple myelonia), among others. In some embodiments, the cancer is a leukemia. In some embodiments, the cancer is acute myeloid leukemia. In some embodiments, the cancer is acute lymphoblastic leukemia (ALL). In some embodiments, the cancer is a solid tumor.

Also disclosed is a method for treating an infectious disease in a subject, comprising administering to the subject an effective amount of a composition disclosed herein that contains a PD-1 specific aptamer. For example, the infectious disease can be selected from the group consisting of tuberculosis, human immunodeficiency virus (HIV), and Acute Hepatitis C Virus (HCV).

The details of one or more embodiments of the invention are set forth in the accompanying drawings and the description below. Other features, objects, and advantages of the invention will be apparent from the description and drawings, and from the claims.

### BRIEF DESCRIPTION OF DRAWINGS

FIG. 1 shows an immunoblot analysis after immunoprecipitation of PD-1 from the lysates of human leukemic cell lines using anti-PD1 antibody. The cells were treated with 50 IU of interferon for 24 hrs.

FIG. 2 shows PAGE analysis for PCR amplified PCR from representative iterative rounds of Selection.

FIG. 3 shows Clustal X multiple sequence analysis of converged sequences from round 6 and 7. A high repetitive element TTGGTGTGGTGGG (SEQ ID NO:64) is present in all converged sequences.

FIG. 4 shows a phylogenetic tree representing proximity relations among the converged sequences after 7th round of selection.

FIG. 5 shows five hundred pmoles each of six selected aptamers were separately mixed with streptavidin magnetic beads and incubated for an hour. Unbound aptamers were remove through washes and such beads were further incubated with above HEL cell lysate and unbound protein were thoroughly washed and bound protein on beads was subjected to SDS-PAGE followed by western analysis using PD-1 specific antibodies.

FIG. 6A shows the binding of PD-1 fluoroscein aptamer to HEL cells by flow cytometry. FIG. 6B shows the fluorescence microscopy of PD-1 aptamer in HEL cells.

FIG. 7 is a schematic of the predicted secondary structure of PD-1 specific aptamers and G-quadruplex in conserved G-rich repeat.

FIG. 8 shows the activation of allogeneic MLR stimulated by PD-1 antibody and PD-1 aptamers at day 7.

FIG. 9A shows aptamer fpf2 (10-150-373) binding to PD1. FIG. 9B shows aptamer fpf2 binding to PD1 (lysate).

FIG. 10 is a schematic representation SELEX methodology to screen PD-1 specific aptamers.

FIG. 11 shows the isolation and preparation of PD-1 protein for SELEX.

FIG. 12 shows the competitive binding assay between selected DNA aptamers to PD-1 in the presence and absence of PD-1 antibody.

FIGS. 13A-13D show the validation of binding specificity of selected DNA aptamers to PD-1 protein.

FIGS. 14A-14B show the filter binding assay to validate PD-1 aptamer binding to PD-1 protein.

FIGS. 15A-15C show the validation of binding specificity of selected DNA aptamers to PD-1 protein in leukemic patient samples by western and immunoprecipitation analysis.

FIG. 16 shows the screening and characterization of biological activity of PD-1 and its ligand interaction using a cell-based assay in the context of anti PD-1 DNA aptamer.

FIG. 17 shows the aptamer stability that was tested in 96% of humans AB serum at different time points.

FIG. 18 shows the aptamer stability that was tested in 80% of humans AB serum at different time points.

FIG. 19 shows the activation of allogeneic MLR stimulated by PD-1 antibody and PD-1 aptamers at day 1.

FIG. 20 shows the activation of allogeneic MLR stimulated by PD-1 antibody and PD-1 aptamers at day 3.

FIGS. 21A-21C shows the functional activity of PD-1 aptamer was characterized in comparison with PD-1 antibody by ELISA for IFNγ cytokine analysis.

FIGS. 22A-22C shows the functional activity of PD-1 aptamer was characterized in comparison with PD-1 antibody by ELISA for IL-2 cytokine analysis.

FIG. 23 shows the activation of mixed lymphocyte reaction (MLR) with CD4, stimulated by PD-1 antibody and PD-1 aptamers at days 1, 3, 5, and 7.

FIG. 24 shows confocal microscopy analysis of CD4+ T cells (MLR assay) for PD1 and PD-L1 expression control, PD-1 and PD-L1 antibodies.

FIG. 25 shows confocal microscopy analysis of CD4+ T cells (MLR assay) for PD1 and PD-L1 expression control, PD1 aptamers and PD-L1 antibodies.

FIG. 26 shows the activation of mixed lymphocyte reaction (MLR) with CD4, stimulated by PD-1 antibody and PD-1 aptamers at days 1, 3, and 5.

FIG. 27 shows the functional activity of PD-1 aptamer was characterized in comparison with PD-1 antibody by ELISA for IFNγ cytokine analysis at day 1 (MLR with CD4).

FIG. 28 shows the functional activity of PD-1 aptamer was characterized in comparison with PD-1 antibody by ELISA for IFNγ cytokine analysis at day 3 (MLR with CD4).

FIG. 29 shows the functional activity of PD-1 aptamer was characterized in comparison with PD-1 antibody by ELISA for IFNγ cytokine analysis at day 5 (MLR with CD4).

FIG. 30 shows the functional activity of PD-1 aptamer was characterized in comparison with PD-1 antibody by ELISA for IL-2 cytokine analysis at day 1 (MLR with CD4).

FIG. 31 shows the functional activity of PD-1 aptamer was characterized in comparison with PD-1 antibody by ELISA for IL-2 cytokine analysis at day 3 (MLR with CD4).

FIG. 32 shows the functional activity of PD-1 aptamer was characterized in comparison with PD-1 antibody by ELISA for IL-2 cytokine analysis at day 5 (MLR with CD4).

FIG. 33 shows the activation of mixed lymphocyte reaction (MLR) with CD8, stimulated by PD-1 antibody and PD-1 aptamers at days 1, 3, and 5.

FIG. 34 shows the functional activity of PD-1 aptamer was characterized in comparison with PD-1 antibody by ELISA for IFNγ cytokine analysis at day 1 (MLR with CD8).

FIG. 35 shows the functional activity of PD-1 aptamer was characterized in comparison with PD-1 antibody by ELISA for IFNγ cytokine analysis at day 3 (MLR with CD8).

FIG. 36 shows the functional activity of PD-1 aptamer was characterized in comparison with PD-1 antibody by ELISA for IFNγ cytokine analysis at day 5 (MLR with CD8).

FIG. 37 shows the functional activity of PD-1 aptamer was characterized in comparison with PD-1 antibody by ELISA for IL-2 cytokine analysis at day 1 (MLR with CD8).

FIG. 38 shows the functional activity of PD-1 aptamer was characterized in comparison with PD-1 antibody by ELISA for IL-2 cytokine analysis at day 3 (MLR with CD8).

FIG. 39 shows the functional activity of PD-1 aptamer was characterized in comparison with PD-1 antibody by ELISA for IL-2 cytokine analysis at day 5 (MLR with CD8).

FIG. 40 shows PD1 NFATc luciferase reporter in Jurkat with TCR/PD-L1 HEK.

FIG. 41 shows the Incucyte target cell lysis activity.

FIG. 42 shows a schematic for improving stability of the aptamer with pegylation.

FIG. 43 shows the mean plasma concentrations of Cy3 pegylated apatamer compared to control.

FIG. 44 shows a schematic representation of in vivo experiment using HNSG-SGM3mice.

FIG. 45 shows the mean tumor volume in the H-NSG HEL92.1.7 xenograft model.

FIG. 46 shows the median tumor volume in the H-NSG HEL92.1.7 xenograft model.

FIG. 47 shows the mean tumor volume in the H-NSG HEL92.1.7 xenograft model out to 13 days.

### DETAILED DESCRIPTION

Acute myeloid leukemia is characterized by an abnormally high number of immature white blood cells of myeloid origin. It affects 20,000 people annually and accounts for 10,000 deaths, mostly in adults. It is generally a disease seen in an aged population (65+) but is the second most common form of cancer in children after acute lymphoblastic leukemia. Recognition of tumor antigens on the cancer cells by the immune system is one of the most important steps in the clearing of tumor cells. However, cancer cells can develop an extraordinary ability to escape the immune response by expressing some proteins that activate immune checkpoints. Such molecules include PD-1, CTLA-4 and their cognate ligands that effectively inhibit T-cell activation and subsequently escape immune surveillance. Immune checkpoints are a built-in mechanism to prevent autoimmunity. Also, proteolytic processing during antigen presentation by macrophages and dendritic cells has an important implication in the regulation of the immune response, as well as auto immunity. This highly orchestrated biological phenomenon is critical for the establishment of adaptive immunity. Deregulation in any step of antigen processing could lead to the development of self-reactive T cells or immune evasion of tumors. Recently, several therapeutic antibodies have been developed that target PD-1/PD-L1 axis to activate the T-cell response against tumors in humans and have been FDA approved.

Disclosed herein are PD-1 specific DNA aptamers (Anti-PD1-Apt) that mimic therapeutic antibodies targeting the PD-1/PD-L1 axis, which can elicit an active host immune response by restoration of T-cell activation in cancer patients. Aptamers are small molecule ligands composed of short, single-stranded oligonucleotides ranging from 30 to 60 bases in length. Similar to protein antibodies, oligonucleotide aptamers specifically recognize and bind to their targets with high affinity on the basis of their unique 3-dimensional structures. Disclosed herein are DNA aptamers that specifically block interaction of human PD1 and its ligand PDL1. The inventors have demonstrated robust anti-PD1 blockade in Mixed Lymphocyte Reaction (MLR) along with the induction of Th1 cytokines Interferon-gamma and IL-2 in CD8+ T cells.

Reference will now be made in detail to representative embodiments of the invention. While the invention will be described in conjunction with the enumerated embodiments, it will be understood that the invention is not intended to be limited to those embodiments. On the contrary, the invention is intended to cover all alternatives, modifications, and equivalents that may be included within the scope of the present invention as defined by the claims.

One skilled in the art will recognize many methods and materials similar or equivalent to those described herein, which could be used in and are within the scope of the practice of the present invention. The present invention is in no way limited to the methods and materials described.

Unless defined otherwise, technical and scientific terms used herein have the same meaning as commonly understood by one of ordinary skill in the art(s) to which this invention belongs. Although any methods, devices, and materials similar or equivalent to those described herein can be used in the practice or testing of the invention, the preferred methods, devices and materials are now described.

All publications, published patent documents, and patent applications cited in this disclosure are indicative of the level of skill in the art(s) to which the disclosure pertains. All publications, published patent documents, and patent applications cited herein are hereby incorporated by reference to the same extent as though each individual publication, published patent document, or patent application was specifically and individually indicated as being incorporated by reference.

As used in this disclosure, including the appended claims, the singular forms “a,” “an,” and “the” include plural references, unless the content clearly dictates otherwise, and are used interchangeably with “at least one” and “one or more.” Thus, reference to “an aptamer” includes mixtures of aptamers, and the like.

As used herein, the term “about” represents an insignificant modification or variation of the numerical value such that the basic function of the item to which the numerical value relates is unchanged. In some embodiments, the term “about” can include values within 20% (for example, 20%, 10%, 5%, 1%) of the numerical value.

As used herein, the terms “comprises,” “comprising,” “includes,” “including,” “contains,” “containing,” and any variations thereof, are intended to cover a non-exclusive inclusion, such that a process, method, product-by-process, or composition of matter that comprises, includes, or contains an element or list of elements does not include only those elements but may include other elements not expressly listed or inherent to such process, method, product-by-process, or composition of matter.

The term “aptamer” refers to oligonucleic acid or peptide molecules that bind to a specific target molecule. These molecules are generally selected from a random sequence pool. The selected aptamers are capable of adapting unique tertiary structures and recognizing target molecules with high affinity and specificity. A “nucleic acid aptamer” is a DNA or RNA oligonucleic acid that binds to a target molecule via its conformation, and thereby inhibits or suppresses functions of such molecule. A nucleic acid aptamer may be constituted by DNA, RNA, or a combination thereof. A “peptide aptamer” is a combinatorial protein molecule with a variable peptide sequence inserted within a constant scaffold protein. Identification of peptide aptamers is typically performed under stringent yeast dihybrid conditions, which enhances the probability for the selected peptide aptamers to be stably expressed and correctly folded in an intracellular context.

Nucleic acid aptamers are typically oligonucleotides ranging from 10-150 bases (or for example, from 15-50 bases) in length that fold into defined secondary and tertiary structures, such as stem-loops or G-quartets. Nucleic acid aptamers preferably bind the target molecule with a K_{d} less than 10⁻⁶, 10⁻⁸, 10⁻¹⁰, or 10⁻¹². Nucleic acid aptamers can also bind the target molecule with a very high degree of specificity. It is preferred that the nucleic acid aptamers have a K_{d} with the target molecule at least 10, 100, 1000, 10,000, or 100,000-fold lower than the K_{d} of other non-targeted molecules.

Nucleic acid aptamers are typically isolated from complex libraries of synthetic oligonucleotides by an iterative process of adsorption, recovery and reamplification. For example, nucleic acid aptamers may be prepared using the SELEX (Systematic Evolution of Ligands by Exponential Enrichment) method. The SELEX method involves selecting an RNA molecule bound to a target molecule from an RNA pool composed of RNA molecules each having random sequence regions and primer-binding regions at both ends thereof, amplifying the recovered RNA molecule via RT-PCR, performing transcription using the obtained cDNA molecule as a template, and using the resultant as an RNA pool for the subsequent procedure. Such procedure is repeated several times to several tens of times to select RNA with a stronger ability to bind to a target molecule. The base sequence lengths of the random sequence region and the primer binding region are not particularly limited. In general, the random sequence region contains about 20 to 80 bases and the primer binding region contains about 15 to 40 bases. Specificity to a target molecule may be enhanced by prospectively mixing molecules similar to the target molecule with RNA pools and using a pool containing RNA molecules that did not bind to the molecule of interest. An RNA molecule that was obtained as a final product by such technique is used as an RNA aptamer. Representative examples of how to make and use aptamers to bind a variety of different target molecules can be found in U.S. Pat. Nos. 5,476,766, 5,503,978, 5,631,146, 5,731,424, 5,780,228, 5,792,613, 5,795,721, 5,846,713, 5,858,660, 5,861,254, 5,864,026, 5,869,641, 5,958,691, 6,001,988, 6,011,020, 6,013,443, 6,020,130, 6,028,186, 6,030,776, and 6,051,698. An aptamer database containing comprehensive sequence information on aptamers and unnatural ribozymes that have been generated by in vitro selection methods is available at aptamer.icmb.utexas.edu.

A nucleic acid aptamer generally has higher specificity and affinity to a target molecule than an antibody. Accordingly, a nucleic acid aptamer can specifically, directly, and firmly bind to a target molecule. Since the number of target amino acid residues necessary for binding may be smaller than that of an antibody, for example, a nucleic acid aptamer is superior to an antibody, when selective suppression of functions of a given protein among highly homologous proteins is intended.

Non-modified nucleic acid aptamers are cleared rapidly from the bloodstream, with a half-life of minutes to hours, mainly due to nuclease degradation and clearance from the body by the kidneys, a result of the aptamer's inherently low molecular weight. This rapid clearance can be an advantage in applications such as in vivo diagnostic imaging. However, several modifications, such as 2′-fluorine-substituted pyrimidines, polyethylene glycol (PEG) linkage, etc. are available to increase the serum half-life of aptamers to the day or even week time scale.

Another approach to increase the nuclease resistance of aptamers is to use a Spiegelmer. Spiegelmers are ribonucleic acid (RNA)-like molecules built from the unnatural L-ribonucleotides. Spiegelmers are therefore the stereochemical mirror images (enantiomers) of natural oligonucleotides. Like other aptamers, Spiegelmers are able to bind target molecules such as proteins. The affinity of Spiegelmers to their target molecules often lies in the pico-to nanomolar range and is thus comparable to antibodies. In contrast to other aptamers, Spiegelmers have high stability in blood serum since they are less susceptible to be cleaved hydrolytically by enzymes. Nonetheless, they are excreted by the kidneys in a short time due to their low molar mass. Unlike other aptamers, Spiegelmers may not be directly produced by the SELEX method. This is because L-nucleic acids are not amenable to enzymatic methods, such as polymerase chain reaction. Instead, the sequence of a natural aptamer identified by the SELEX method is determined and then used in the artificial synthesis of the mirror image of the natural aptamer.

A “PD-1 aptamer” is an aptamer that specifically binds to PD-1 and disrupts the interaction between Programmed Death-1 (PD-1) and Programmed Death Ligand-1 (PD-L1) under physiological conditions.

As used herein, a “SOMAmer” or Slow Off-Rate Modified Aptamer refers to an aptamer (including an aptamer comprising at least one nucleotide with a hydrophobic modification) with an off-rate (t½) of >30 minutes, >60 minutes, >90 minutes, >120 minutes, >150 minutes, >180 minutes, >210 minutes, or >240 minutes. In some embodiments, SOMAmers are generated using the improved SELEX methods described in U.S. Pat. No. 7,947,447, entitled “Method for Generating Aptamers with Improved Off-Rates”.

As used herein, the term “nucleotide” refers to a ribonucleotide or a deoxyribonucleotide, or a modified form thereof, as well as an analog thereof. Nucleotides include species that include purines (e.g., adenine, hypoxanthine, guanine, and their derivatives and analogs) as well as pyrimidines (e.g., cytosine, uracil, thymine, and their derivatives and analogs).

As used herein, “nucleic acid,” “oligonucleotide,” and “polynucleotide” are used interchangeably to refer to a polymer of nucleotides and include DNA, RNA, DNA/RNA hybrids and modifications of these kinds of nucleic acids, oligonucleotides and polynucleotides, wherein the attachment of various entities or moieties to the nucleotide units at any position are included. The terms “polynucleotide,” “oligonucleotide,” and “nucleic acid” include double- or single-stranded molecules as well as triple-helical molecules. Nucleic acid, oligonucleotide, and polynucleotide are broader terms than the term aptamer and, thus, the terms nucleic acid, oligonucleotide, and polynucleotide include polymers of nucleotides that are aptamers but the terms nucleic acid, oligonucleotide, and polynucleotide are not limited to aptamers.

As used herein, the terms “modify”, “modified”, “modification”, and any variations thereof, when used in reference to an oligonucleotide, means that at least one of the four constituent nucleotide bases (i.e., A, G, T/U, and C) of the oligonucleotide is an analog or ester of a naturally occurring nucleotide. In some embodiments, the modified nucleotide confers nuclease resistance to the oligonucleotide. In some embodiments, the modified nucleotides lead to predominantly hydrophobic interactions of aptamers with protein targets resulting in high binding efficiency and stable co-crystal complexes. A pyrimidine with a substitution at the C-5 position is an example of a modified nucleotide. Modifications can include backbone modifications, methylations, unusual base-pairing combinations such as the isobases isocytidine and isoguanidine, and the like. Modifications can also include 3′ and 5′ modifications, such as capping. Other modifications can include substitution of one or more of the naturally occurring nucleotides with an analog, internucleotide modifications such as, for example, those with uncharged linkages (e.g., methyl phosphonates, phosphotriesters, phosphoamidates, carbamates, etc.) and those with charged linkages (e.g., phosphorothioates, phosphorodithioates, etc.), those with intercalators (e.g., acridine, psoralen, etc.), those containing chelators (e.g., metals, radioactive metals, boron, oxidative metals, etc.), those containing alkylators, and those with modified linkages (e.g., alpha anomeric nucleic acids, etc.). Further, any of the hydroxyl groups ordinarily present on the sugar of a nucleotide may be replaced by a phosphonate group or a phosphate group; protected by standard protecting groups; or activated to prepare additional linkages to additional nucleotides or to a solid support. The 5′ and 3′ terminal OH groups can be phosphorylated or substituted with amines, organic capping group moieties of from about 1 to about 20 carbon atoms, polyethylene glycol (PEG) polymers in some embodiments ranging from about 10 to about 80 kDa, PEG polymers in some embodiments ranging from about 20 to about 60 kDa, or other hydrophilic or hydrophobic biological or synthetic polymers. In some embodiments, modifications are of the C-5 position of pyrimidines. These modifications can be produced through an amide linkage directly at the C-5 position or by other types of linkages. Polynucleotides can also contain analogous forms of ribose or deoxyribose sugars that are generally known in the art, including 2′-0-methyl-, 2′-0-allyl, 2′-fluoro- or 2′-azido-ribose, carbocyclic sugar analogs, a-anomeric sugars, epimeric sugars such as arabinose, xyloses or lyxoses, pyranose sugars, furanose sugars, sedoheptuloses, acyclic analogs and abasic nucleoside analogs such as methyl riboside. As noted above, one or more phosphodiester linkages may be replaced by alternative linking groups. These alternative linking groups include embodiments wherein phosphate is replaced by P(0)S (“thioate”), P(S)S (“dithioate”), (0)NR2 (“amidate”), P(0)R, P(0)OR′, CO or CH2 (“formacetal”), in which each R or R′ is independently H or substituted or unsubstituted alkyl (1-20 C) optionally containing an ether (-0-) linkage, aryl, alkenyl, cycloalky, cycloalkenyl or araldyl. Not all linkages in a polynucleotide need be identical. Substitution of analogous forms of sugars, purines, and pyrimidines can be advantageous in designing a final product, as can alternative backbone structures like a polyamide backbone, for example.

Nucleotides can be modified either before or after synthesis of an oligonucleotide. A sequence of nucleotides in an oligonucleotide may be interrupted by one or more non- nucleotide components. A modified oligonucleotide may be further modified after polymerization, such as, for example, by conjugation with any suitable labeling component.

As used herein, the term “at least one pyrimidine,” when referring to modifications of a nucleic acid, refers to one, several, or all pyrimidines in the nucleic acid, indicating that any or all occurrences of any or all of C, T, or U in a nucleic acid may be modified or not.

As used herein, A, C, G, U and T denote dA, dC, dG, dU and dT respectively, unless otherwise specified.

As used herein, “protein” is used synonymously with “peptide,” “polypeptide,” or “peptide fragment.” A “purified” polypeptide, protein, peptide, or peptide fragment is substantially free of cellular material or other contaminating proteins from the cell, tissue, or cell-free source from which the amino acid sequence is obtained, or substantially free from chemical precursors or other chemicals when chemically synthesized.

As used herein “cancer” means a disease or condition involving unregulated and abnormal cell growth. Some examples of common cancers are bladder cancer, lung cancer, breast cancer, melanoma, colon and rectal cancer, lymphoma, endometrial cancer, pancreatic cancer, liver cancer, renal cancer, prostate cancer, leukemia (for example, acute myeloid leukemia (AML), acute lymphoblastic leukemia (ALL)) and thyroid cancer. In some embodiments, the cancer is a hematologic malignancy, which includes, but is not limited to, leukemia, lymphoma, myeloid disorder, lymphoid disorder, myelodysplastic syndrome (MDS), myeloproliferative disease (MPD), mast cell disorder, and myeloma (e.g., multiple myeloma), among others. In some embodiments, the cancer is a solid tumor.

As used herein a “linker” is a molecular entity that connects two or more molecular entities through covalent bond or non-covalent interactions and can allow spatial separation of the molecular entities in a manner that preserves the functional properties of one or more of the molecular entities. A linker can also be known as a spacer. Appropriate linker sequences will be readily ascertained by those of skill in the art based upon the present disclosure.

As used herein, a linker can comprise one or more molecules or sub-components, selected from the group including, but not limited to, a polynucleotide, a polypeptide, a peptide nucleic acid, a locked nucleic acid, an oligosaccharide, a polysaccharide, an antibody, an affybody, an antibody mimic, an aliphatic, aromatic or heteroaromatic carbon molecule, a polyethylene glycol (PEG) molecule, a cell receptor, a ligand, a lipid, any fragment or derivative of these structures, any combination of the foregoing, or any other chemical structure or component.

In some such embodiments, the disclosed aptamer comprises at least one modified nucleoside comprising a hydrophobic nucleobase modification. Further, in some such embodiments, the hydrophobic nucleobase modification is a modified pyrimidine. In some embodiments, each modified pyrimidine may be independently selected from 5-(N-benzylcarboxyamide) -2′-deoxyuridine (BndU), 5-(N-benzylcarboxyamide)-2′-0-methyluridine, 5-(N-benzylcarboxyamide)-2′-fluorouridine, 5-(N-phenethylcarboxyamide)-2′-deoxyuridine (PedU), 5-(N-thiophenylmethylcarboxyamide)-2′-deoxyuridine (ThdU), 5-(N-isobutylcarboxyamide) -2′-deoxyuridine (iBudU), 5-(N-isobutylcarboxyamide)-2′-0-methyluridine, 5-(N-isobutylcarboxyamide)-2′-fluorouridine, 5-(N-tryptaminocarboxyamide)-2′-deoxyuridine (TrpdU), 5-(N-tryptaminocarboxyamide)-2′-0-methyluridine, 5-(N-tryptaminocarboxyamide) -2′-fluorouridine, 5-(N-[1-(3-trimethylamonium) propyl]carboxyamide)-2′-deoxyuridine chloride, 5-(N-naphthylmethylcarboxyamide)-2′-deoxyuridine (NapdU), 5-(N-naphthylmethylcarboxyamide)-2′-0-methyluridine, 5-(N-naphthylmethylcarboxyamide) -2′-fluorouridine, and 5-(N-[1-(2,3-dihydroxypropyl)]carboxyamide)-2′-deoxyuridine).

In some embodiments, the disclosed aptamer can comprises a C2-C50 linker or spacer, which may be a backbone comprising a chain of 2 to 50 carbon atoms (C2-C50) (saturated, unsaturated, straight chain, branched or cyclic), 0 to 10 aryl groups, 0 to 10 heteroaryl groups, and 0 to 10 heterocyclic groups, optionally comprising an ether (-0-) linkage, (e.g., one or more alkylene glycol units, including but not limited to one or more ethylene glycol units -0—(CH2CH20)—; one or more 1,3-propane diol units —O—(α¾α¾α¾0)—, etc.); an amine (—NH—) linkage; an amide (—NC(O)—) linkage; and a thioether (—S—) linkage; etc.; wherein each backbone carbon atom may be independently unsubstituted (i.e., comprising —H substituents) or may be substituted with one or more groups selected from a d to C3 alkyl, —OH, —NH2, —SH, -0-(Ci to C6 alkyl), —S—(Ci to C6 alkyl), halogen, —OC(0)(Ci to C6 alkyl), —NH—(Ci to C6 alkyl), and the like.

In some embodiments, a C2-C50 linker is a C2-C20 linker, a C2-C10 linker, a C2-C8 linker, a C2-C6 linker, a C2-C5 linker, a C2-C4 linker, or a C3 linker, wherein each carbon may be independently substituted as described above.

In some embodiments, one or more nucleosides of the disclosed aptamer comprise a modification selected from a 2′-position sugar modification (such as a 2′-amino (2′-NH2), a 2′-fluoro (2′-F), or a 2′-0-methyl (2′-0Me)), a modification at a cytosine exocyclic amine, an internucleoside linkage modification, and a 5-methyl-cytosine. In some embodiments, a PDGF aptamer comprises a 3′ cap, a 5′ cap, and/or an inverted deoxythymidine at the 3′ terminus.

In some embodiments, the disclosed aptamer comprises at least one modified internucleoside linkage. In some embodiments, at least one, at least two, at least three, at least four, or at least five internucleoside linkages are phosphorothioate linkages.

In some embodiments, the disclosed aptamer has a sequence selected from the sequences shown in Table 2 (SEQ ID NOS: 6 to 63). In some embodiments, the disclosed aptamer has a sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to the sequences shown in Table 2 (SEQ ID NOs: 6 to 63).

The terms “sequence identity”, “percent sequence identity”, “percent identity”, “% identical”, “% identity”, and variations thereof, when used in the context of two nucleic acid sequences, are used interchangeably to refer to the number of nucleotide bases that are the same in a query nucleic acid or a portion of a query nucleic acid, when it is compared and aligned for maximum correspondence to a reference nucleic acid, divided by either (1) the number of nucleotide bases in the query sequence between and including the most 5′ corresponding (i.e., aligned) nucleotide base and the most 3′ corresponding (i.e., aligned) nucleotide base, or (2) the total length of the reference sequence, whichever is greater. Exemplary alignment of sequences for comparison can be conducted, e.g., by the local homology algorithm of Smith and Waterman (1981) Adv. Appl. Math. 2:482, by the homology alignment algorithm of Needleman and Wunsch (1970) J. Mol. Biol. 48:443, by the search for similarity method of Pearson and Lipman (1988) Proc. Nat'l. Acad. Sci. USA 85:2444, by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally, Ausubel, F. M. et al. (1987), Current Protocols in Molecular Biology, Greene Publishing Assoc. and Wiley-Inter science). [00135] One example of an algorithm that is suitable for determining percent sequence identity is the algorithm used in the basic local alignment search tool (hereinafter “BLAST”), see, e.g. Altschul et al. (1990) J. Mol. Biol. 215:403 and Altschul et al. (1997) Nucleic Acids Res. 15:3389. Software for performing BLAST analyses is publicly available through the National Center for Biotechnology Information (hereinafter “NCBI”). The default parameters used in determining sequence identity using the software available from NCBI, e.g., BLASTN (for nucleotide sequences) are described in McGinnis et al. (2004) Nucleic Acids Res. 32:W20.

As used herein, when describing the percent identity of a nucleic acid, such as an aptamer, the sequence of which is at least, for example, about 95% identical to a reference nucleotide sequence, it is intended that the nucleic acid sequence is identical to the reference sequence except that the nucleic acid sequence may include up to five point mutations per each 100 nucleotides of the reference nucleic acid sequence. In other words, to obtain a desired nucleic acid sequence, the sequence of which is at least about 95% identical to a reference nucleic acid sequence, up to 5% of the nucleotides in the reference sequence may be deleted or substituted with another nucleotide, or some number of nucleotides up to 5% of the total number of nucleotides in the reference sequence may be inserted into the reference sequence (referred to herein as an insertion). These mutations of the reference sequence to generate the desired sequence may occur at the 5′ or 3′ terminal positions of the reference nucleotide sequence or anywhere between those terminal positions, interspersed either individually among nucleotides in the reference sequence or in one or more contiguous groups within the reference sequence. Further, it is intended that a nucleotide base is considered “identical” for the purposes of determining percent identity, when the nucleotide base (1) is the same as the nucleotide base in the reference sequence, or (2) is derived from the nucleotide base in the reference sequence, or (3) is derived from the same nucleotide base from which the nucleotide base in the reference sequence is derived. For example, 5-methyl cytosine is considered to be “identical” to cytosine for the purposes of calculating percent identity.

In some aspects, disclosed is a single-stranded DNA aptamer, comprising the formula

n-L-n-Z-n-R-n,

wherein “L” comprises the nucleic acid sequence TACCTGATAGCGTATGCGA (SEQ ID NO:2), or a nucleic acid sequence having at least 80% sequence identity to SEQ ID NO:2, or a fragment thereof at least 15 nucleotides in length,

wherein “Z” comprises the nucleic acid sequence GGGxTTGGTGTGGTGGG (SEQ ID NO:1), wherein “x” is A, T, or C,

wherein “R” comprises the nucleic acid sequence CTCTCAGTAGGTGCATAAGCG (SEQ ID NO:3), or a nucleic acid sequence having at least 80% sequence identity to SEQ ID NO:3, or a fragment thereof at least 15 nucleotides in length, and

wherein each “n” is independently any 0 to 10 nucleotides,

wherein the DNA aptamer specifically binds to PD-1 and disrupts the interaction between Programmed Death-1 (PD-1) and Programmed Death Ligand-1 (PD-L1) under physiological conditions.

In some embodiments, the DNA aptamer comprises a nucleic acid sequence selected from the group consisting of SEQ ID NO:6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, and 63.

In some embodiments, the DNA aptamer comprises a nucleic acid sequence having at least 80% sequence identity to a nucleic acid sequence selected from the group consisting of SEQ ID NO:6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, and 63. In some embodiments, the DNA aptamer comprises SEQ ID NO:12. In some embodiments, the DNA aptamer comprises SEQ ID NO:18.

In some embodiments, the disclosed aptamer comprises at least 12, at least 13, at least 14, at least 15, at least 16, at least 17, at least 18, at least 19, at least 20, at least 21, at least 21, at least 22, at least 23, at least 24, at least 25, at least 26, at least 27, at least 28, at least 29, or at least 30 contiguous nucleotides of a nucleic acid sequence selected from the group consisting of SEQ ID NO:6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, and 63.

In some embodiments, the disclosed aptamer comprises 12, 13, 14, 15, 16, 17, 18, 19, 20, 21, 21, 22, 23, 24, 25, 26, 27, 28, 29, or 30 contiguous nucleotides of a nucleic acid sequence selected from the group consisting of SEQ ID NO:6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, and 63.

In some embodiments, the DNA comprises a nucleic acid sequence selected from the group consisting of SEQ ID NO:6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, and 63, wherein the sequence comprises the nucleic acid sequence fragment located between SEQ ID NO:2 and SEQ ID NO:3 in each of the SEQ ID NOs:6 to 63.

The disclosed aptamer can contain any number of nucleotides in addition to the PD-1 binding region. In various embodiments, the disclosed aptamer can include up to about 100 nucleotides, up to about 95 nucleotides, up to about 90 nucleotides, up to about 85 nucleotides, up to about 80 nucleotides, up to about 75 nucleotides, up to about 70 nucleotides, up to about 65 nucleotides, up to about 60 nucleotides, up to about 55 nucleotides, up to about 50 nucleotides, up to about 45 nucleotides, up to about 40 nucleotides, up to about 35 nucleotides, up to about 30 nucleotides, up to about 25 nucleotides, or up to about 20 nucleotides.

The disclosed aptamer can be selected to have any suitable dissociation constant (Kd) for PD-1. In some embodiments, a PD-1 aptamer has a dissociation constant (Kd) for PD-1 of less than 30 nM, less than 25 nM, less than 20 nM, less than 15 nM, less than 10 nM, less than 9 nM, less than 8 nM, less than 7 nM, less than 6 nM, less than 5 nM, less than 4 nM, less than 3 nM, less than 2 nM, or less than 1 nM. Dissociation constants may be determined with a binding assay using a multi-point titration and fitting the equation y=(max−min)(Protein)/(Kd+Protein)+min. In some embodiments, the PD-1 aptamer is an aptamer with a Kd that is less than or equal to the Kd of an aptamer shown in Table 2.

In some aspects, disclosed is a single-stranded DNA aptamer, comprising the formula

n-L-n-Z-n-R-n,

wherein “L” comprises the nucleic acid sequence TACCTGATAGCGTATGCGA (SEQ ID NO:2), or a nucleic acid sequence having at least 80% sequence identity to SEQ ID NO:2, or a fragment thereof at least 15 nucleotides in length,

wherein “Z” comprises the nucleic acid sequence GGGxTTGGTGTGGTGGG (SEQ ID NO:1), or a nucleic acid sequence having at least 80% sequence identity to SEQ ID NO:1, or a fragment thereof at least 15 nucleotides in length; wherein “x” is A, T, or C;

wherein “R” comprises the nucleic acid sequence CTCTCAGTAGGTGCATAAGCG (SEQ ID NO:3), or a nucleic acid sequence having at least 80% sequence identity to SEQ ID NO:3, or a fragment thereof at least 15 nucleotides in length, and

wherein each “n” is independently any 0 to 10 nucleotides,

wherein the DNA aptamer specifically binds to PD-1 and disrupts the interaction between Programmed Death-1 (PD-1) and Programmed Death Ligand-1 (PD-L1) under physiological conditions.

In some aspects, disclosed is a single-stranded DNA aptamer, comprising the nucleic acid sequence GGGxTTGGTGTGGTGGG (SEQ ID NO:1), wherein “x” is A, T, or C. In some aspects, disclosed is a single-stranded DNA aptamer, comprising the nucleic acid sequence GGGxTTGGTGTGGTGGG (SEQ ID NO:1), or a nucleic acid sequence having at least 80% sequence identity to SEQ ID NO:1; wherein “x” is A, T, or C. In some aspects, disclosed is a single-stranded DNA aptamer, comprising the nucleic acid sequence GGGxTTGGTGTGGTGGG (SEQ ID NO:1), or a nucleic acid sequence having at least 80% sequence identity to SEQ ID NO:1, or a fragment thereof at least 15 nucleotides in length; wherein “x” is A, T, or C.

In some aspects, disclosed is a single-stranded DNA aptamer, comprising the formula

n-Z-n,

wherein “Z” comprises the nucleic acid sequence GGGxTTGGTGTGGTGGG (SEQ ID NO:1), wherein “x” is A, T, or C, and

wherein each “n” is independently any 0 to 10 nucleotides,

wherein the DNA aptamer specifically binds to PD-1 and disrupts the interaction between Programmed Death-1 (PD-1) and Programmed Death Ligand-1 (PD-L1) under physiological conditions.

In some aspects, disclosed is a single-stranded DNA aptamer, comprising the formula

n-Z-n,

wherein “Z” comprises the nucleic acid sequence GGGxTTGGTGTGGTGGG (SEQ ID NO:1), or a nucleic acid sequence having at least 80% sequence identity to SEQ ID NO:1, or a fragment thereof at least 15 nucleotides in length; wherein “x” is A, T, or C;

wherein each “n” is independently any 0 to 10 nucleotides,

wherein the DNA aptamer specifically binds to PD-1 and disrupts the interaction between Programmed Death-1 (PD-1) and Programmed Death Ligand-1 (PD-L1) under physiological conditions.

In some embodiments, n is independently selected from 0, 1, 2, 3, 4, 5, 6, 7, 8, 9, or 10 nucleotides. In some embodiments, n is 0 nucleotides. In some embodiments, n is 1 nucleotide. In some embodiments, n is 2 nucleotides. In some embodiments, n is 3 nucleotides. In some embodiments, n is 4 nucleotides. In some embodiments, n is 5 nucleotides. In some embodiments, n is 6 nucleotides. In some embodiments, n is 7 nucleotides. In some embodiments, n is 8 nucleotides. In some embodiments, n is 9 nucleotides. In some embodiments, n is 10 nucleotides. In some embodiments, n is independently selected from 0 to 50 nucleotides.

In some aspects, disclosed is a single-stranded DNA aptamer, comprising the formula

n-L-n-Z-m-R-n,

wherein “L” comprises the nucleic acid sequence TACCTGATAGCGTATGCGA (SEQ ID NO:2), or a nucleic acid sequence having at least 80% sequence identity to SEQ ID NO:2, or a fragment thereof at least 15 nucleotides in length,

wherein “Z” comprises the nucleic acid sequence GGGxTTGGTGTGGTGGG (SEQ ID NO:1), or a nucleic acid sequence having at least 80% sequence identity to SEQ ID NO:1, or a fragment thereof at least 15 nucleotides in length; wherein “x” is A, T, or C;

wherein “R” comprises the nucleic acid sequence CTCTCAGTAGGTGCATAAGCG (SEQ ID NO:3), or a nucleic acid sequence having at least 80% sequence identity to SEQ ID NO:3, or a fragment thereof at least 15 nucleotides in length,

wherein each “n” is independently any 0 to 10 nucleotides,

wherein “m” is any 0 to 50 nucleotides,

wherein the DNA aptamer specifically binds to PD-1 and disrupts the interaction between Programmed Death-1 (PD-1) and Programmed Death Ligand-1 (PD-L1) under physiological conditions.

Pharmaceutical Compositions Comprising Aptamers and Aptamer Constructs

In some embodiments, pharmaceutical compositions comprising at least one aptamer or aptamer construct described herein and at least one pharmaceutically acceptable carrier are provided. Suitable carriers are described in “Remington: The Science and Practice of Pharmacy, Twenty-first Edition,” published by Lippincott Williams & Wilkins, which is incorporated herein by reference.

The aptamers described herein can be utilized in any pharmaceutically acceptable dosage form, including, but not limited to, injectable dosage forms, liquid dispersions, gels, aerosols, ointments, creams, lyophilized formulations, dry powders, tablets, capsules, controlled release formulations, fast melt formulations, delayed release formulations, extended release formulations, pulsatile release formulations, mixed immediate release and controlled release formulations, etc. Specifically, the aptamers described herein can be formulated: (a) for administration selected from any of oral, pulmonary, intravenous, intraarterial, intrathecal, intra-articular, rectal, ophthalmic, colonic, parenteral, intracisternal, intravaginal, intraperitoneal, local, buccal, nasal, and topical administration; (b) into a dosage form selected from any of liquid dispersions, gels, aerosols, ointments, creams, tablets, sachets and capsules; (c) into a dosage form selected from any of lyophilized formulations, dry powders, fast melt formulations, controlled release formulations, delayed release formulations, extended release formulations, pulsatile release formulations, and mixed immediate release and controlled release formulations; or (d) any combination thereof.

Solutions or suspensions used for parenteral, intradermal, or subcutaneous application can comprise one or more of the following components: (1) a sterile diluent such as water for injection, saline solution, fixed oils, polyethylene glycols, glycerine, propylene glycol or other synthetic solvents; (2) antibacterial agents such as benzyl alcohol or methyl parabens; (3) antioxidants such as ascorbic acid or sodium bisulfite; (4) chelating agents such as ethylenediaminetetraacetic acid; (5) buffers such as acetates, citrates or phosphates; and (5) agents for the adjustment of tonicity such as sodium chloride or dextrose. The pH can be adjusted with acids or bases, such as hydrochloric acid or sodium hydroxide. A parenteral preparation can be enclosed in ampoules, disposable syringes or multiple dose vials made of glass or plastic.

Pharmaceutical compositions suitable for injectable use may include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL (BASF, Parsippany, N.J.) or phosphate buffered saline (PBS). In all cases, the composition should be sterile and should be fluid to the extent that easy syringability exists. The pharmaceutical composition should be stable under the conditions of manufacture and storage and should be preserved against the contaminating action of microorganisms such as bacteria and fungi. The term “stable”, as used herein, means remaining in a state or condition that is suitable for administration to a subject.

The carrier can be a solvent or dispersion medium, including, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion, and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol or sorbitol, and inorganic salts such as sodium chloride, in the composition. Prolonged absorption of the injectable compositions can be brought about by including in the composition an agent which delays absorption, for example, aluminum monostearate and gelatin.

Sterile injectable solutions can be prepared by incorporating the active reagent (e.g., an aptamer, and/or an aptamer construct) in an appropriate amount in an appropriate solvent with one or a combination of ingredients enumerated above, as desired, followed by filtered sterilization. Generally, dispersions are prepared by incorporating at least one aptamer, and/or aptamer construct into a sterile vehicle that contains a basic dispersion medium and any other desired ingredient. In the case of sterile powders for the preparation of sterile injectable solutions, exemplary methods of preparation include vacuum drying and freeze-drying, both of which will yield a powder of an aptamer, and/or an aptamer construct plus any additional desired ingredient from a previously sterile-filtered solution thereof.

In some embodiments, an aptamer, and/or an aptamer construct is formulated for intravitreal injection. Suitable formulations for intravitreal administration are described, e.g., in. Ocular drug delivery is discussed, e.g., in Rawas-Qalaji et al. (2012) Curr. Eye Res. 37: 345; Bochot et al. (2012) J. Control Release 161:628; Yasukawa et al. (2011) Recent Pat. Drug Deliv. Formul. 5: 1; and Doshi et al. (2011) Semin Ophthalmol. 26: 104. In some embodiments, a pharmaceutical composition comprising an aptamer, and/or an aptamer construct is administered by intravitreal injection once per week, once per two weeks, once per three weeks, once per four weeks, once per five weeks, once per six weeks, once per seven weeks, once per eight weeks, once per nine weeks, once per 10 weeks, once per 11 weeks, once per 12 weeks, or less often than once per 12 weeks. [00177] Oral compositions generally include an inert diluent or an edible carrier. They can be enclosed, for example, in gelatin capsules or compressed into tablets.

For the purpose of oral therapeutic administration, the aptamer, and/or aptamer construct can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition.

For administration by inhalation, the compounds are delivered in the form of an aerosol spray from a pressured container or dispenser that contains a suitable propellant, e.g., a gas such as carbon dioxide, a nebulized liquid, or a dry powder from a suitable device. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active reagents are formulated into ointments, salves, gels, or creams, as generally known in the art. The reagents can also be prepared in the form of suppositories (e.g., with conventional suppository bases such as cocoa butter and other glycerides) or retention enemas for rectal delivery.

In some embodiments, an aptamer, and/or an aptamer construct is prepared with a carrier that will protect against rapid elimination from the body. For example, a controlled release formulation can be used, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc.

Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

Additionally, suspensions of an aptamer, and/or an aptamer construct may be prepared as appropriate oily injection suspensions. Suitable lipophilic solvents or vehicles include fatty oils, such as sesame oil, or synthetic fatty acid esters, such as ethyl oleate, triglycerides, or liposomes. Non-lipid polycationic amino polymers may also be used for delivery. Optionally, the suspension may also include suitable stabilizers or agents to increase the solubility of the compounds and allow for the preparation of highly concentrated solutions.

In some cases, it may be especially advantageous to formulate oral or parenteral compositions in dosage unit form for ease of administration and uniformity of dosage.

Dosage unit form as used herein refers to physically discrete units suited as unitary dosages for the subject to be treated; each unit containing a predetermined quantity of an aptamer and/or aptamer construct calculated to produce the desired therapeutic effect in association with the required pharmaceutical carrier. The specification for the dosage unit forms of aptamers and/or constructs described herein are dictated by and directly dependent on the characteristics of the particular aptamer and/or aptamer construct and the particular therapeutic effect to be achieved, and the limitations inherent in the art of compounding such an active agent for the treatment of individuals.

Pharmaceutical compositions comprising at least one aptamer, and/or aptamer construct can include one or more pharmaceutical excipients. Examples of such excipients include, but are not limited to, binding agents, filling agents, lubricating agents, suspending agents, sweeteners, flavoring agents, preservatives, buffers, wetting agents, disintegrants, effervescent agents, and other excipients. Such excipients are known in the art. Exemplary excipients include: (1) binding agents which include various celluloses and cross-linked polyvinylpyrrolidone, microcrystalline cellulose, such as Avicel PH101 and Avicel PHI 02, silicified microcrystalline cellulose (ProSolv SMCC™), gum tragacanth and gelatin; (2) filling agents such as various starches, lactose, lactose monohydrate, and lactose anhydrous; (3) disintegrating agents such as alginic acid, Primogel, corn starch, lightly crosslinked polyvinyl pyrrolidone, potato starch, maize starch, and modified starches, croscarmellose sodium, cross-povidone, sodium starch glycolate, and mixtures thereof; (4) lubricants, including agents that act on the flowability of a powder to be compressed, and including magnesium stearate, colloidal silicon dioxide, such as Aerosil 200, talc, stearic acid, calcium stearate, and silica gel; (5) glidants such as colloidal silicon dioxide; (6) preservatives, such as potassium sorbate, methylparaben, propylparaben, benzoic acid and its salts, other esters of parahydroxybenzoic acid such as butylparaben, alcohols such as ethyl or benzyl alcohol, phenolic compounds such as phenol, or quaternary compounds such as benzalkonium chloride; (7) diluents such as pharmaceutically acceptable inert fillers, such as microcrystalline cellulose, lactose, dibasic calcium phosphate, saccharides, and/or mixtures of any of the foregoing; examples of diluents include microcrystalline cellulose, such as Avicel PH101 and Avicel PHI 02; lactose such as lactose monohydrate, lactose anhydrous, and Pharmatose DCL21; dibasic calcium phosphate such as Emcompress; mannitol; starch; sorbitol; sucrose; and glucose; (8) sweetening agents, including any natural or artificial sweetener, such as sucrose, saccharin sucrose, xylitol, sodium saccharin, cyclamate, aspartame, and acesulfame; (9) flavoring agents, such as peppermint, methyl salicylate, orange flavoring, Magnasweet (trademark of MAFCO), bubble gum flavor, fruit flavors, and the like; and (10) effervescent agents, including effervescent couples such as an organic acid and a carbonate or bicarbonate. Suitable organic acids include, for example, citric, tartaric, malic, fumaric, adipic, succinic, and alginic acids and anhydrides and acid salts. Suitable carbonates and bicarbonates include, for example, sodium carbonate, sodium bicarbonate, potassium carbonate, potassium bicarbonate, magnesium carbonate, sodium glycine carbonate, L-lysine carbonate, and arginine carbonate. Alternatively, only the sodium bicarbonate component of the effervescent couple may be present.

In various embodiments, the formulations described herein are substantially pure. As used herein, “substantially pure” means the active ingredient (e.g., an aptamer, and/or an aptamer construct) is the predominant species present (i.e., on a molar basis it is more abundant than any other individual species in the composition). In some embodiments, a substantially purified fraction is a composition wherein the active ingredient comprises at least about 50 percent (on a molar basis) of all macromolecular species present. Generally, a substantially pure composition will include more than about 80% of all macromolecular species present in the composition. In various embodiments, a substantially pure composition will include at least about 85%, at least about 90%, at least about 95%, or at least about 99% of all macromolecular species present in the composition. In various embodiments, the active ingredient is purified to homogeneity (contaminant species cannot be detected in the composition by conventional detection methods) wherein the composition consists essentially of a single macromolecular species.

Kits Comprising Aptamers and Aptamer Constructs

The present disclosure provides kits comprising any of the aptamers, and/or aptamer constructs described herein. Such kits can comprise, for example, (1) at least one aptamer, and/or aptamer constructs; and (2) at least one pharmaceutically acceptable carrier, such as a solvent or solution. Additional kit components can optionally include, for example: (1) any of the pharmaceutically acceptable excipients identified herein, such as stabilizers, buffers, etc., (2) at least one container, vial or similar apparatus for holding and/or mixing the kit components; and (3) delivery apparatus.

Methods of Treatment

The present disclosure provides methods of preventing or treating (e.g., alleviating one or more symptoms of) medical conditions through the use of a PD-1 aptamer or aptamer construct. The methods comprise administering a therapeutically effective amount of such aptamer and/or aptamer construct to a subject in need thereof. The described aptamers can also be used for prophylactic therapy. In some embodiments, the aptamer and/or aptamer construct is administered orally or intravenously.

In some aspects, disclosed herein is a method for inhibiting the interaction between PD-1 and PD-L1 in a subject, comprising administering to the subject an effective amount of a DNA aptamer (or composition comprising the DNA aptamer) as described herein, wherein the DNA aptamer specifically binds to PD-1 and disrupts the interaction between Programmed Death-1 (PD-1) and Programmed Death Ligand-1 (PD-L1) under physiological conditions.

In some embodiments, the subject is suffering from a cancer selected from the group consisting of melanoma, non-small-cell lung cancer (NSCLC), renal cell carcinoma (RCC), and bladder cancer. In some embodiments, the subject is suffering from acute myeloid leukemia. In some embodiments, the subject is a human.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder. The term treatment includes administration of any of the compositions disclosed herein.

The term “subject” or “host” refers to any individual who is the target of administration or treatment. The subject can be a vertebrate, for example, a mammal. Thus, the subject can be a human or veterinary patient. The term “patient” refers to a subject under the treatment of a clinician, e.g., physician.

The term “therapeutically effective” refers to the amount of the composition used is of sufficient quantity to ameliorate one or more causes or symptoms of a disease or disorder. Such amelioration only requires a reduction or alteration, not necessarily elimination.

In some embodiments, the disclosed compounds or pharmaceutically acceptable salts thereof, or prodrugs, can be administered in combination with other treatments that improve or eradicate the disease conditions as described above. Compositions including the disclosed aptamers and/or aptamer constructs may contain, for example, more than one aptamer. In some examples, a composition containing one or more aptamers is administered in combination with another useful cardiovascular agent or anticancer agent or antifibrotic agent etc. In general, the currently available dosage forms of the known therapeutic agents for use in such combinations will be suitable.

“Combination therapy” (or “co-therapy”) includes the administration of an aptamer and/or aptamer construct composition and at least one second agent as part of a specific treatment regimen intended to provide the beneficial effect from the co-action of these therapeutic agents. The beneficial effect of the combination includes, but is not limited to, pharmacokinetic or pharmacodynamic co-action resulting from the combination of therapeutic agents. Administration of these therapeutic agents in combination typically is carried out over a defined time period (usually minutes, hours, days or weeks depending upon the combination selected).

The dosage regimen utilizing the aptamers and/or aptamer constructs is selected in accordance with a variety of factors, including, for example, type, species, age, weight, gender and medical condition of the subject; the severity of the condition to be treated; the route of administration; the renal and hepatic function of the subject; and the particular aptamer and/or aptamer constructs or salts thereof employed. An ordinarily skilled physician or veterinarian can readily determine and prescribe the effective amount of the composition required to prevent, counter or arrest the progress of the condition.

In some embodiments, the methods disclosed herein are used to treat a cancer in a subject, for example, a hematologic malignancy. In some embodiments, the hematologic malignancy includes, but is not limited to, leukemia, lymphoma, myeloid disorder, lymphoid disorder, myelodysplastic syndrome (MDS), myeloproliferative disease (MPD), mast cell disorder, and myeloma (e.g., multiple myeloma), among others. In some embodiments, the cancer is a leukemia. In some embodiments, the cancer is acute myeloid leukemia. In some embodiments, the cancer is acute lymphoblastic leukemia (ALL).

As contemplated herein, the cancer treated can be a primary tumor or a metastatic tumor. In one aspect, the methods described herein are used to treat a solid tumor, for example, melanoma, lung cancer (including lung adenocarcinoma, basal cell carcinoma, squamous cell carcinoma, large cell carcinoma, bronchioloalveolar carcinoma, bronchogenic carcinoma, non-small-cell carcinoma, small cell carcinoma, mesothelioma); breast cancer (including ductal carcinoma, lobular carcinoma, inflammatory breast cancer, clear cell carcinoma, mucinous carcinoma, serosal cavities breast carcinoma); colorectal cancer (colon cancer, rectal cancer, colorectal adenocarcinoma); anal cancer; pancreatic cancer (including pancreatic adenocarcinoma, islet cell carcinoma, neuroendocrine tumors); prostate cancer; prostate adenocarcinoma; ovarian carcinoma (ovarian epithelial carcinoma or surface epithelial-stromal tumor including serous tumor, endometrioid tumor and mucinous cystadenocarcinoma, sex-cord-stromal tumor); liver and bile duct carcinoma (including hepatocellular carcinoma, cholangiocarcinoma, hemangioma); esophageal carcinoma (including esophageal adenocarcinoma and squamous cell carcinoma); oral and oropharyngeal squamous cell carcinoma; salivary gland adenoid cystic carcinoma; bladder cancer; bladder carcinoma; carcinoma of the uterus (including endometrial adenocarcinoma, ocular, uterine papillary serous carcinoma, uterine clear-cell carcinoma, uterine sarcomas, leiomyosarcomas, mixed mullerian tumors); glioma, glioblastoma, medulloblastoma, and other tumors of the brain; kidney cancers (including renal cell carcinoma, clear cell carcinoma, Wilm's tumor); cancer of the head and neck (including squamous cell carcinomas); cancer of the stomach (gastric cancers, stomach adenocarcinoma, gastrointestinal stromal tumor); testicular cancer; germ cell tumor; neuroendocrine tumor; cervical cancer; carcinoids of the gastrointestinal tract, breast, and other organs; signet ring cell carcinoma; mesenchymal tumors including sarcomas, fibrosarcomas, haemangioma, angiomatosis, haemangiopericytoma, pseudoangiomatous stromal hyperplasia, myofibroblastoma, fibromatosis, inflammatory myofibroblastic tumor, lipoma, angiolipoma, granular cell tumor, neurofibroma, schwannoma, angiosarcoma, liposarcoma, rhabdomyosarcoma, osteosarcoma, leiomyoma, leiomysarcoma, skin, including melanoma, cervical, retinoblastoma, head and neck cancer, pancreatic, brain, thyroid, testicular, renal, bladder, soft tissue, adexal gland, urethra, cancers of the penis, myxosarcoma, chondrosarcoma, osteosarcoma, chordoma, malignant fibrous histiocytoma, lymphangiosarcoma, mesothelioma, squamous cell carcinoma; epidermoid carcinoma, malignant skin adnexal tumors, adenocarcinoma, hepatoma, hepatocellular carcinoma, renal cell carcinoma, hypernephroma, cholangiocarcinoma, transitional cell carcinoma, choriocarcinoma, seminoma, embryonal cell carcinoma, glioma anaplastic; glioblastoma multiforme, neuroblastoma, medulloblastoma, malignant meningioma, malignant schwannoma, neurofibrosarcoma, parathyroid carcinoma, medullary carcinoma of thyroid, bronchial carcinoid, pheochromocytoma, Islet cell carcinoma, malignant carcinoid, malignant paraganglioma, melanoma, Merkel cell neoplasm, cystosarcoma phylloide, salivary cancers, thymic carcinomas, and cancers of the vagina among others.

In some aspects, disclosed herein is a method for inhibiting the interaction between PD-1 and PD-L1 in a cell, comprising administering to the cell a DNA aptamer (or composition comprising the DNA aptamer) as described herein, wherein the DNA aptamer specifically binds to PD-1 and disrupts the interaction between Programmed Death-1 (PD-1) and Programmed Death Ligand-1 (PD-L1) under physiological conditions.

In some embodiments, the DNA aptamer is an isolated or purified DNA aptamer. In some embodiments, the DNA aptamer is a non-naturally occurring sequence. In some embodiments, the DNA aptamer is a synthetic DNA.

A number of embodiments of the invention have been described. Nevertheless, it will be understood that various modifications may be made without departing from the spirit and scope of the invention. Accordingly, other embodiments are within the scope of the following claims.

### EXAMPLES

### Example 1

### Identification of Programmed Cell Death-1 (PD-1) Specific DNA Aptamer Sequences and their Utility as Immunotherapeutic Agents in Diagnosis and Treatment of Cancers

Disclosed are sequences of high affinity DNA aptamers selected against native human PD1 protein using SELEX procedure with few modifications.

Library Design: A combinatorial DNA library with 25 bases random region flanking 19 base primer on either end were purchased from Integrated DNA technologies. Table 1 shows the forward and reverse primers used for PCR amplification of selected aptamer pools. Additionally, reverse primer with 5′ biotin was utilized for single strand DNA preparation using streptavidin magnetic beads during iterative rounds of screening. 5′ FAM on forward primer was helpful in tracking the selected strand during selection and ssDNA preparation.

TABLE 1

| Sequence | 5′ | |
|---|---|---|
| Name | Modification | Sequence |
| AMLIB-1 | None | tacctgatagcgtatgcga- |
| | | NNNNNNNNNNNNNNNNNNNNNNNNN- |
| | | ctctcagtaggtgcataagcg |
| | | (SEQ ID NO: 4) |
| AMLIB-2 | None | tacctgatagcgtatgcga |
| | | (SEQ ID NO: 2) |
| AMLIB-3 | /56-FAM/ | tacctgatagcgtatgcga |
| | | (SEQ ID NO: 2) |
| AMLIB-4 | /5Biosg/ | cgcttatgcacctactgagag |
| | | (SEQ ID NO: 5) |
| AMLIB-5 | None | cgcttatgcacctactgagag |
| | | (SEQ ID NO: 5) |

TABLE 2

| Sequence | |
|---|---|
| Name | Sequence |
| 6-164-408. | ccgtatacat acctgatagc gtatgcgagg gtttggtgtg gtggggctct |
| 03-2-1-0 | tacctctcag taggtgcata agcgcatgca tgc (SEQ ID NO: 6) |
| 20-101-251. | ccgtatacat acctgatagc gtatgcgaga gggtttggtg tggtggggct |
| 29-2-2-4 | ctcctctcag taggtgcata agcgcatgca tgc (SEQ ID NO: 7) |
| 23-94-233. | ccgtatacat acctgatagc gtatgcgagg gtttggtgtg gtggggctct |
| 87-2-3-3 | tacctctcag taggtgcata agcggcatgc ata (SEQ ID NO: 8) |
| 46-57-141. | ccgtatacat acctgatagc gtatgcgagg gattggtgtg gtggggctcg |
| 82-2-4-5 | cggctctcag taggtgcata agcgcatgca tgc (SEQ ID NO: 9) |
| 56-47-116. | ccgtatacat acctgatagc gtatgcgagg gtttggtgtg gtggggctcg |
| 94-2-5-3 | gtactctcag taggtgcata agcgcatgca tgc (SEQ ID NO: 10) |
| 7-163-405. | ccgtatacat acctgatagc gtatgcgagg aagggcttgg tgtggtgggg |
| 54-3-1-0 | cttctctcag taggtgcata agcgcatgca tgc (SEQ ID NO: 11) |
| 15-120-298. | ccgtatacat acctgatagc gtatgcgagg agagggattg gtgtggtggg |
| 56-3-2-3 | gctctctcag taggtgcata agcgcatgca tgc (SEQ ID NO: 12) |
| 21-100-248. | ccgtatacat acctgatagc gtatgcgagg aagggcttgg tgtggtgggg |
| 80-3-3-3 | cttctctcag taggtgcata agcggcatgc ata (SEQ ID NO: 13) |
| 49-52-129. | ccgtatacat acctgatagc gtatgcgacc aagggcttgg tgtggtgggg |
| 38-3-4-2 | cttctctcag taggtgcata agcgcatgca tgc (SEQ ID NO: 14) |
| 70-39-97. | ccgtatacat acctgatagc gtatgcgacc aagggcttgg tgtggtgggg |
| 03-3-5-5 | cttctctcag taggtgcata agcggcatgc ata (SEQ ID NO: 15) |
| 97-30-74. | ccgtatacat acctgatagc gtatgcgagg aagggcttgg tgtggtgggg |
| 64-3-6-1 | gcttctctca gtaggtgcat aagcgcatgc atgc (SEQ ID NO: 16) |
| 9-153-380. | ccgtatacat acctgatagc gtatgcgata taggagggtt tggtgtggtg |
| 66-5-1-0 | ggctctcagt aggtgcataa gcgttttggg atcctgggtt cgtcctctca |
| | gtaggtgcat aagcgcatgc atgc (SEQ ID NO: 17) |
| 10-150-373. | ccgtatacat acctgatagc gtatgcgagg aagtgggctt ggtgtggtgg |
| 20-6-1-0 | gcttctctca gtaggtgcat aagcgcatgc atgc (SEQ ID NO: 18) |
| 27-80-199. | ccgtatacat acctgatagc gtatgcgagg aagtgggctt ggtgtggtgg |
| 04-6-2-3 | gcttctctca gtaggtgcat aagcggcatg cata (SEQ ID NO: 19) |
| 31-73-181. | ccgtatacat acctgatagc gtatgcgagg agagggattg gtgtggtggg |
| 62-6-3-5 | gctctctcag taggtgcata agcgcatgca tgc (SEQ ID NO: 20) |
| 51-50-124. | ccgtatacat acctgatagc gtatgcgacc aagggcttgg tgtggtgggg |
| 40-6-4-5 | cttctctcag taggtgcata agcgcatgca tgc (SEQ ID NO: 21) |
| 13-130-323. | ccgtatacat acctgatagc gtatgcgata taggagggtt tggtgtggtg |
| 44-7-1-0 | gggctctcag taggtgcata agcgttttgg gatcctgggt tcgtcctctc |
| | agtaggtgca taagcgcatg catgcatc (SEQ ID NO: 22) |
| 17-114-283. | ccgtatacat acctgatagc gtatgcgagg gtttggtgtg gtggggctct |
| 63-8-1-0 | tacctctcag taggtgcata agcgcatgca tgc (SEQ ID NO: 23) |
| 22-99-246. | ccgtatacat acctgatagc gtatgcgagg gtttggtgtg gtggggctct |
| 31-8-2-3 | tacctctcag taggtgcata agcggcatgc ata (SEQ ID NO: 24) |
| 24-92-228. | ccgtatacat acctgatagc gtatgcgaga gggtttggtg tggtggggct |
| 89-8-3-4 | ctcctctcag taggtgcata agcgcatgca tgc (SEQ ID NO: 25) |
| 97-30-74. | ccgtatacat acctgatagc gtatgcgagg gtttggtgtg gtggggctcg |
| 64-8-4-3 | gtactctcag taggtgcata agcgcatgca tgc (SEQ ID NO: 26) |
| 26-87-216. | ggcatatgtt acctgatagc gtatgcgagg agggtttggt gtggtggggc |
| 45-10-1-0 | tcgctctcag taggtgcata agcgcatgca tgc (SEQ ID NO: 27) |
| 61-45-111. | ggcatatgtt acctgatagc gtatgcgagg agggtttggt gtggtggggc |
| 96-10-2-3 | tcgctctcag taggtgcata agcggcatgc ata (SEQ ID NO: 28) |
| 90-32-79. | ggcatatgtt acctgatagc gtatgcgagg aagggcttgg tgtggtgggg |
| 62-10-3-4 | cttctctcag taggtgcata agcgcatgca tgc (SEQ ID NO: 29) |
| 109-28-69. | ggcatatgtt acctgatagc gtatgcgagg agagggattg gtgtggtggg |
| 66-10-4-5 | gctctctcag taggtgcata agcgcatgca tgc (SEQ ID NO: 30) |
| 28-78-194. | ccgtatacat acctgatagc gtatgcgaga gggtttggtg tggtggggct |
| 06-11-1-0 | ctcctctcag taggtgcata agcggcatgc ata (SEQ ID NO: 31) |
| 97-30-74. | ccgtatacat acctgatagc gtatgcgagg gtttggtgtg gtggggctcg |
| 64-11-2-5 | gtactctcag taggtgcata agcggcatgc ata (SEQ ID NO: 32) |
| 33-67-166. | ccgtatacat acctgatagc gtatgcgata taggagggtt tggtgtggtg |
| 69-12-1-0 | gggctctcag taggtgcata agcgcatgca tgc (SEQ ID NO: 33) |
| 36-64-159. | ccgtatacat acctgatagc gtatgcgagg tgggagggat tggtgtggtg |
| 23-12-2-4 | gggctctcag taggtgcata agcgcatgca tgc (SEQ ID NO: 34) |
| 66-43-106. | ccgtatacat acctgatagc gtatgcgacc taggagggtt tggtgtggtg |
| 98-12-3-2 | gggctctcag taggtgcata agcgcatgca tgc (SEQ ID NO: 35) |
| 70-39-97. | ccgtatacat acctgatagc gtatgcgacc taggagggtt tggtgtggtg |
| 03-12-4-5 | gggctctcag taggtgcata agcggcatgc ata (SEQ ID NO: 36) |
| 100-29-72. | ccgtatacat acctgatagc gtatgcgata taggagggtt tggtgtggtg |
| 15-12-5-3 | gggctctcag taggtgcata agcggcatgc ata (SEQ ID NO: 37) |
| 34-65-161. | ccgtatacat acctgatagc gtatgcgagg tgggagggat tggtgtggtg |
| 72-13-1-0 | gggctctcag taggtgcata agcggcatgc ata (SEQ ID NO: 38) |
| 37-62-154. | ccgtatacat acctgatagc gtatgcgagg agagggattg gtgtggtggg |
| 25-13-2-5 | gctctctcag taggtgcata agcggcatgc ata (SEQ ID NO: 39) |
| 40-60-149. | ccgtatacat acctgatagc gtatgcgagg agagggattg gtgtggtggg |
| 28-14-1-0 | gctctctcag taggtgcata agcggcatgc ata (SEQ ID NO: 40) |
| 43-59-146. | ccgtatacat acctgatagc gtatgcgacc aagggcttgg tgtggtgggg |
| 79-14-2-5 | cttctctcag taggtgcata agcggcatgc ata (SEQ ID NO: 41) |
| 47-56-139. | ccgtatacat acctgatagc gtatgcgagg aagtgggctt ggtgtggtgg |
| 33-15-1-0 | gcttctctca gtaggtgcat aagcgttttg ggatcctggg ttcgtcctct |
| | cagtaggtgc ataagcgcat gcatgc (SEQ ID NO: 42) |
| 48-53-131. | ccgtatacat acctgatagc gtatgcgaga gggtttggtg tggtggggct |
| 86-16-1-0 | ctcctctcag taggtgcata agcggcatgc ata (SEQ ID NO: 43) |
| 86-33-82. | ccgtatacat acctgatagc gtatgcgagg gtttggtgtg gtggggctcg |
| 10-16-2-5 | gtactctcag taggtgcata agcggcatgc ata (SEQ ID NO: 44) |
| 50-51-126. | ccgtatacat acctgatagc gtatgcgagg aagggcttgg tgtggtgggg |
| 89-17-1-0 | cttctctcag taggtgcata agcgttttgg gatcctgggt tcgtcctctc |
| | agtaggtgca taagcgcatg catgcatc (SEQ ID NO: 45) |
| 86-33-82. | ccgtatacat acctgatagc gtatgcgagg agagggattg gtgtggtggg |
| 10-17-2-3 | gctctctcag taggtgcata agcgttttgg gatcctgggt tcgtcctctc |
| | agtaggtgca taagcgcatg catgcatc (SEQ ID NO: 46) |
| 113-26-64. | ccgtatacat acctgatagc gtatgcgaga gggtttggtg tggtggggct |
| 69-17-3-5 | ctcctctcag taggtgcata agcgttttgg gatcctgggt tcgtcctctc |
| | agtaggtgca taagcgcatg catgcatc (SEQ ID NO: 47) |
| 54-49-121. | ccgtatacat acctgatagc gtatgcgagg gtttggtgtg gtggggctct |
| 91-18-1-0 | tacctctcag taggtgcata agcgttttgg gatcctgggt tcgtcctctc |
| | agtaggtgca taagcgcatg catgc (SEQ ID NO: 48) |
| 56-47-116. | ccgtatacat acctgatagc gtatgcgagg gtttggtgtg gtggggctct |
| 94-19-1-0 | tacctctcag taggtgcata agcgttttgg gatcctgggt tcgtcctctc |
| | agtaggtgca taagcgcatg catgc (SEQ ID NO: 49) |
| 64-44-109. | ccgtatacat acctgatagc gtatgcgaga gggtttggtg tggtggggct |
| 47-19-2-4 | ctcctctcag taggtgcata agcgttttgg gatcctgggt tcgtcctctc |
| | agtaggtgca taagcgcatg catgc (SEQ ID NO: 50) |
| 70-39-97. | ccgtatacat acctgatagc gtatgcgagg gattggtgtg gtggggctcg |
| 03-20-1-0 | cggctctcag taggtgcata agcggcatgc ata (SEQ ID NO: 51) |
| 70-39-97. | ccgtatacat acctgatagc gtatgcgagg tgggagggat tggtgtggtg |
| 03-21-1-0 | gggctctcag taggtgcata agcggagttc gttcttctgt tgtgtttctc |
| | tctctcagta ggtgcataag cgcatgcatg catc (SEQ ID NO: 52) |
| 70-39-97. | ccgtatacat acctgatagc gtatgcgata taggagggtt tggtgtggtg |
| 03-22-1-0 | ggctctcagt aggtgcataa gcggcatgca ta (SEQ ID NO: 53) |
| 80-35-87. | ccgtatacat acctgatagc gtatgcgata taggagggtt tggtgtggtg |
| 08-22-2-3 | ggctctcagt aggtgcataa gcgcatgcat gc (SEQ ID NO: 54) |
| 85-34-84. | ccgtatacat acctgatagc gtatgcgacc taggagggtt tggtgtggtg |
| 59-22-3-3 | gggctctcag taggtgcata agcggcatgc ata (SEQ ID NO: 55) |
| 79-36-89. | ggcatatgtt acctgatagc gtatgcgaat cgtgagggtt tggtgtggtg |
| 57-24-1-0 | gggctctcag taggtgcata agcgcatgca tgc (SEQ ID NO: 56) |
| 80-35-87. | ggcatatgtt acctgatagc gtatgcgaat cgtgagggtt tggtgtggtg |
| 08-24-2-3 | gggctctcag taggtgcata agcggcatgc ata (SEQ ID NO: 57) |
| 80-35-87. | ccgtatacat acctgatagc gtatgcgagg agagggattg gtgtggtggg |
| 08-25-1-0 | gctctctcag taggtgcata agcgatagcg tatgcgattt gtttcgttcg |
| | gatctgtata ttctctcagt aggtgca (SEQ ID NO: 58) |
| 93-31-77. | ccgtatacat acctgatagc gtatgcgagg tgggagggat tggtgtggtg |
| 13-26-1-0 | ggctctcagt aggtgcataa gcggagttcg ttcttctgtt gtgtttctct |
| | ctctcagtag gtgcataagc gcatgcatgc (SEQ ID NO: 59) |
| 93-31-77. | ccgtatacat acctgatagc gtatgcgatg accctcgttc gtctctctct |
| 13-27-1-0 | cccctctcag tcgttggtag tctcttttgt ctctcagtag gtgcataagc |
| | gcatgcatgc (SEQ ID NO: 60) |
| 100-29-72. | ccgtatacat acctgatagc gtatgcgacc taggagggtt tggtgtggtg |
| 15-28-1-0 | gggctctcag taggtgcata agcgcatgca tgc (SEQ ID NO: 61) |
| 100-29-72. | ccgtatacat acctgatagc gtatgcgatg accctcgttc gtctctctct |
| 15-29-1-0 | cccctctcag tcgttggtag tctcttttgt ctctcagtag gtgcataagc |
| | gcatgcatgc c (SEQ ID NO: 62) |
| 113-26-64. | gcgaggagag ggattggtgt ggtggggctc tctcagtagg tgcataagcg |
| 69-31-1-0 | atagcgtatg cgatttgttt cgttcggatc tgtatattct ctcagtaggt |
| | gcataagcgc atgcatgc (SEQ ID NO: 63) |

Sequences in Table 2 can generally be referred to using the first three numbers in column 1 above. For some sequence names, the first three numbers can be the same, and thus are identified using the additional four numbers as well.

Isolation and preparation of PD1 for SELEX. Endogenous PD1 expression levels were compared in four distinct human cell lines by immunoblot analysis after 6 and 24 hrs of interferon treatment. Based on the expression levels of PD-1 after 24 hrs, two individual cell lines namely HEL 92.1.7 (ATCC) and KG-1 (DSMZ) were selected for further isolation of PD1 individually by immunoprecipitation.

For preparing PD-1 for SELEX screens, 100 μl of 1:1 mixture of protein A and protein G magnetic beads were washed three times with PBS-T and were used to pre-clear the cell lysates and then discarded. Same amount of similarly prepared beads were incubated with 10 ug of anti-PD-1 antibody at 4° C. for 2 hrs. After 3 times PBS-T washes, pre-clear lysates corresponding to total protein of 500 μg from each HEL (ATCC) and KG-1 (DSMZ) cell lines were incubated overnight at 4° C. with antibody coated beads to capture PD-1 on to the surface. Such beads with captured PD-1 were separated from the supernatant and washed 3 times with PBS-T. A small aliquot of such beads were subjected to immunoblot analysis to confirm the presence of captured PD-1 and rest of them were used for SELEX rounds as described below.

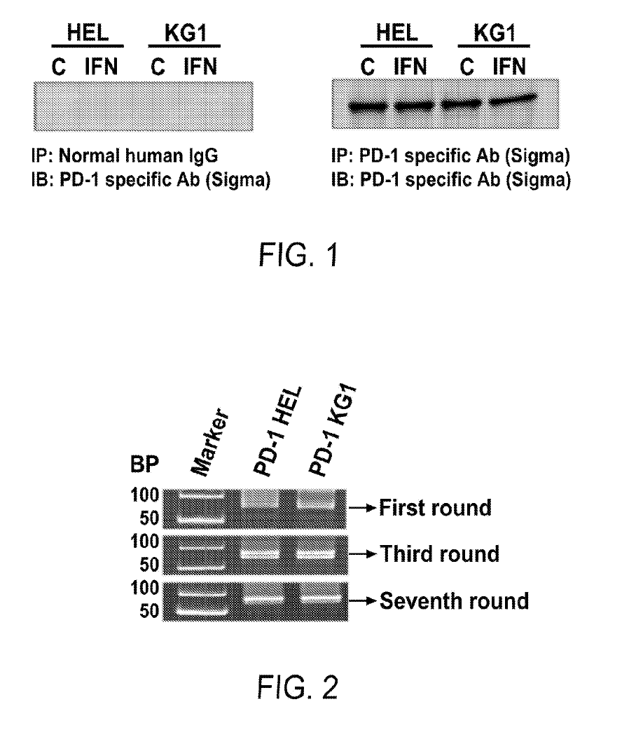

SELEX procedure, Next Generation Sequencing and Data Analysis. One Nano mole of the purchased library was suspended in 50 ml of PBS pH 7.4 containing 2 mM MgCl2 and heated to 95oC for 10 minutes in water bath and then allowed to cool slowly at room temperature. After cooling to room temperature, magnetic beads coated with anti-PD1 antibody were added to the library mix and incubated for 30 minutes at room temperature to remove those aptamers that have high affinity towards magnetic beads as well as anti-PD1 antibody. The beads were removed by magnetic separation and the supernatant containing the reduced library was now incubated with magnetic beads having captured PD-1 and incubated for 1 hour at room temperature. After incubation, the beads were separated and thoroughly washed three times (10 ml each time) with PBS pH 7.4 containing 2 mM MgCl₂. The washed beads were used as template in PCR amplification with primers AMLIB3 and AMLIB4 (see library design). PCR conditions were 95° C. for 3 min initial denaturation; [95° C. for 45 seconds, 54° C. for 45 seconds and 72° C. for 45 seconds]×20 cycles; followed by 72° C. for 5 minutes final extension, FIG. 2. Single strand DNA for next iterative round of selection was prepared by capturing the biotinylated strand (arising out of biotinylated primer) on the strepatividin magnetic beads followed by strand separation in presence of 0.1M NaOH. NaOH was removed by 10K centricon spin filtration from the single stranded DNA prep. Seven rounds of Iterative selection were performed in a similar fashion as described above each time with freshly prepared PD-1 magnetic beads. At each round, an aliquot of selected pool of PCR product was stored for PCR amplification using primers with additional “tag” sequences to allow later identification of their source following next generation sequencing. Such analysis is highly useful to track convergence of binding sequence with the iterative rounds of selection. Next-generation sequencing was performed on a single Ion-torrent 314 chip (SeqWright) after pooling PCR products in equal proportions from all the seven rounds and two different lines each having tracking tags (unique barcode) at 5′ and 3′ positions.

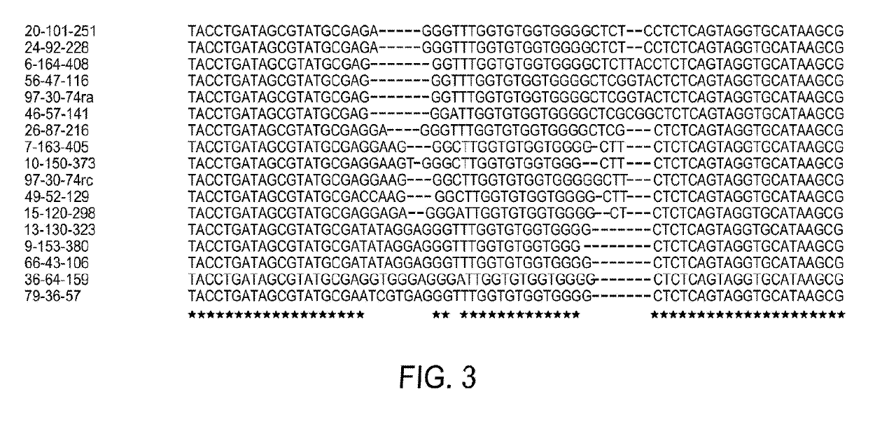

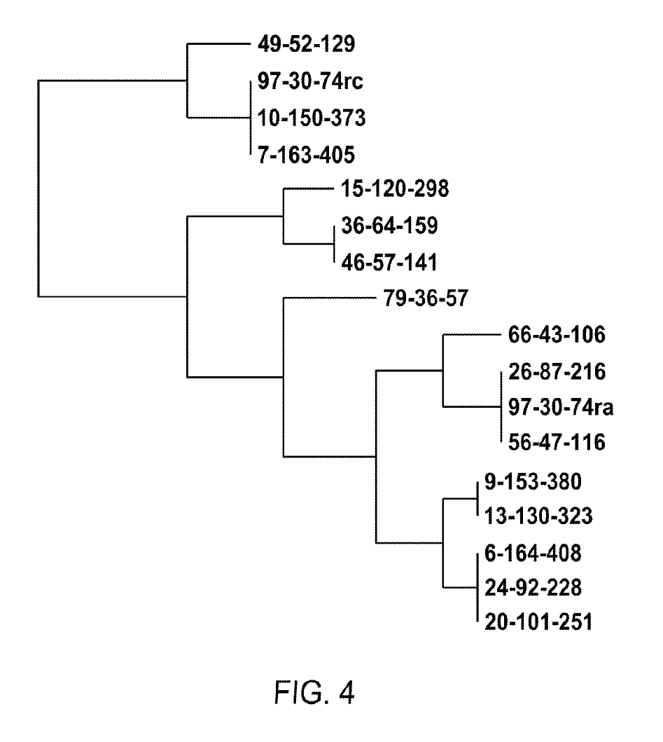

Analysis of the NGS data was be performed using Galaxy project sequence analysis tools and FastAptamer software suite [Alam K K et al. (2015) Molecular Therapy-Nucleic acids 4: e230:1-10]. The software has manual tools to sort and align the sequencing data into clusters and tally each sequence as a function of DNA tag allowing us to identify best binding sequences to PD-1 (FIG. 3). Multiple sequence comparison and dendrograms were further generated using clustal X [Thompson J D et al (1997) Nucleic Acids Res 25: 4876-4882] (FIG. 4).

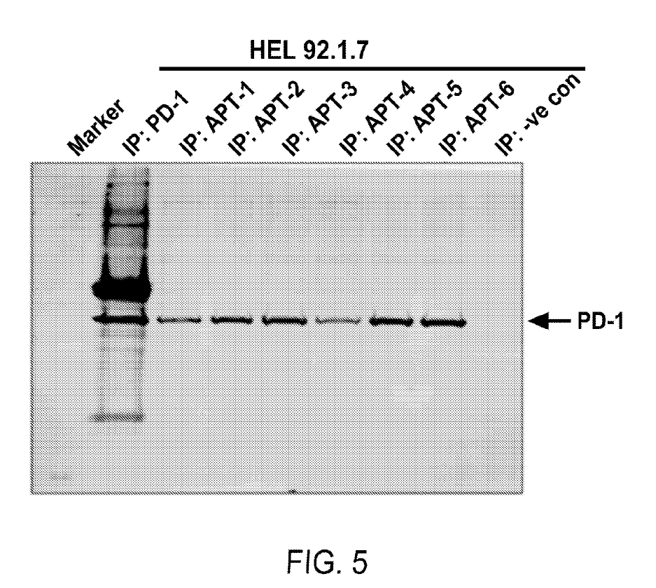

Validation of binding specificity of selected DNA aptamers to PD-1. To determine the ability of selected aptamers to recognize endogenous PD-1, PD-1 pull-down assays were performed from total cell lysate of HEL 92.1.7 (ATCC) using six best represented biotinylated aptamers. The PD-1 protein levels from the pull-down assays were comparable to immunoprecipitated protein using PD1-specific antibodies (FIG. 5).

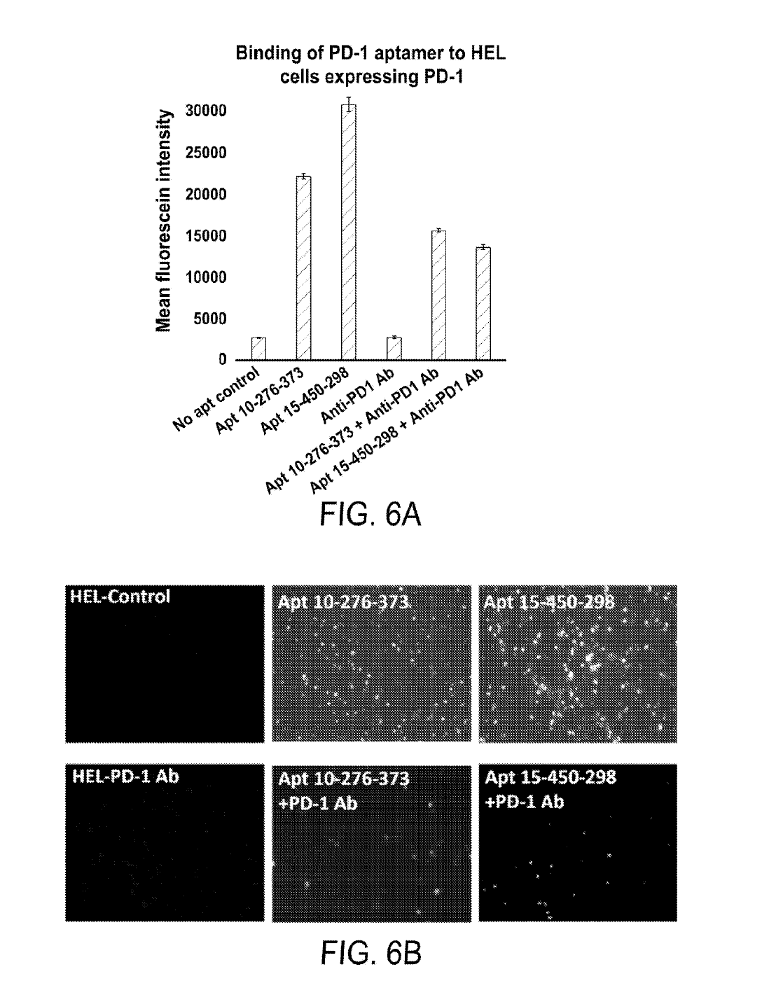

Two selected 15-120-298 and 10-150-373 aptamers were further validated for PD-1 binding by flow cytometry and microscopy. Briefly, the flourescently labeled aptamer were incubated with HEL cells expressing PD-1 and throughly washed to remove the unbound aptamer. The cells were then analyzed by flow cytometry for mean flourescent intensity (FIG. 6A). Also, the cells were examined by flourescence microscopy (FIG. 6B).

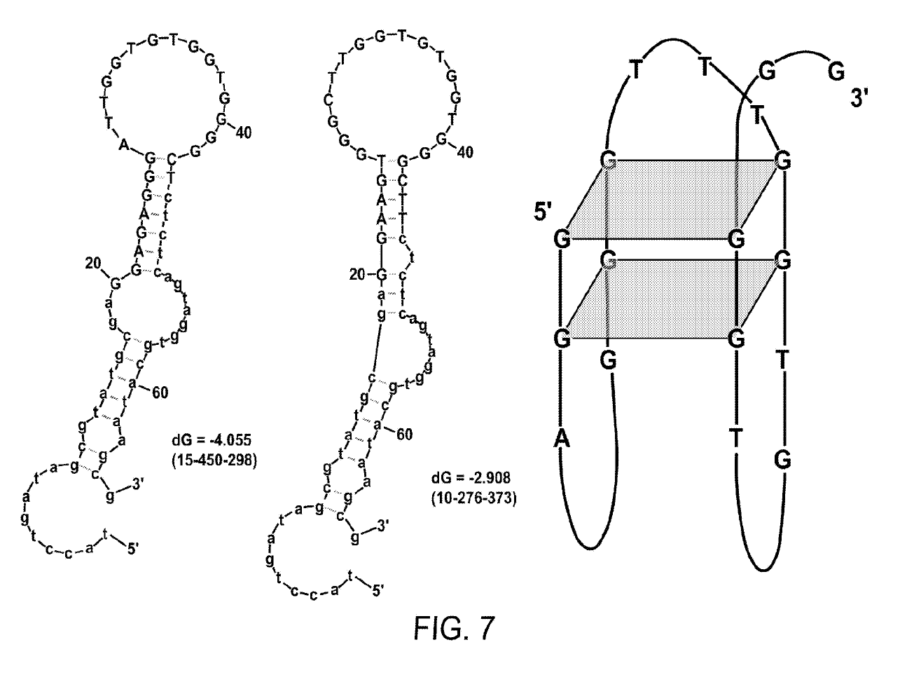

Secondary structures of PD-1 specific aptamers. mfold algorithm was used to predict the secondary structures of selected aptamers. For all the aptamers, the optimal and suboptimal structures had a minimum free energy in range of −1.5 to −4.0 kcal/mole suggesting these structures had low stability (FIG. 7).

Determination of the affinity of aptamers by flow cytometry using mean flourescence intensities. Although, there was an observed mobility shift of aptamers in polyacrylamide gel with HEL cell lysates, the same phenonmenon was not observed with commercially procured purified PD-1 protein due to the fact that these aptamers were selected against the immunoprecipated endogenous PD-1 protein that might be in a complex or in an alternate conformation. Therefore, in order to determine the binding affinity mean flourescently intensity was measured using flow cytometer upon titrating flourescently labeled aptamer to fixed number of cells expressing PD-1. Cells lacking PD1-expression were used as negative control. To determine the binding affinities of the selected PD-1 aptamers towards HEL 92.1.7 and KG-1 cell lines, various concentrations of carboxyfluorescein (FAM) labelled PD-1 specific aptamers and non-specific (negative control) aptamers ranging from 500 nM to 0.1 nM were incubated with either HEL 92.1.7 or KG-1 cells (1×10⁵ cells for each), respectively, for 15 min in 50 μl of binding buffer. Cells were then washed 5-6 times with 100 μl of wash buffer and then re-suspended in 50 μl of binding buffer for analysis via flow cytometry and 1×10⁴ cells were counted for analysis. The cells without aptamers treatment serve as the background control. The specific binding intensity was calculated by subtracting the mean fluorescence intensity from the background intensity. The resulting mean fluorescence intensity of the specific PD-1 aptamers was further used to calculate the equilibrium dissociation constant (Kd). Kd of the fluorescence PD-1 aptamer was plotted using the prism software (GraphPad Software, Inc. USA) which can be fitted as a plot of the mean fluorescence intensity of the specific binding intensity (Y) versus the aptamer concentration(X), Y=BmaxX/(Kd+X), in the assumption of only one binding site (Hung, L.-Y. et al. (2015) Sci. Rep. 5, 10326; Sefah, Ket al. (2010) Nat. Protoc. 5, 1169-1185; Weng, C. H. et al. (2013) Microfluid Nanofluid 14, 753-765).

Determination of the binding affinity of PD-1 aptamers from fluoroscence intensity values by plate reader. To determine the binding affinities of the selected PD-1 aptamers towards HEL 92.1.7 and KG-1 cell lines, various concentrations of carboxyfluorescein (FAM) labelled PD-1 specific aptamers and non-specific aptamers (negative control) ranging from 500 nM to 0.1 nM were incubated with either HEL 92.1.7 or KG-1 cells (1×10⁵ cells for each), respectively, for 15 min in 50 μl of binding buffer. 1×10⁴ cells were then washed 5-6 times with 100 μl of wash buffer and then re-suspended in 50 μl of binding buffer and measured for fluorescence intensity via plate reader (BioTEK). Following experiment would be repeated 3×for reproducibility. The cells without the treatment of aptamers serve as background control. The specific fluorescence intensity was calculated by subtracting the fluorescence intensity values from the background intensity. The resulting fluorescence intensity of the specific PD-1 aptamers was further used to calculate the equilibrium dissociation constant (Kd). Kd of the fluorescence PD-1 aptamer was obtained by using the prism software (GraphPad Software, Inc. USA) which can be fitted as a plot of the mean fluorescence intensity of the specific binding intensity (Y) versus the aptamer concentration(X), Y=BₘₐₓX/K_{d}+X), in the assumption of only one binding site.

PD-1 specific aptamers and antibody binding to HEL 92.1.7 and KG-1 cells by confocal microscopy imaging. To visualize the binding location of the selected PD-1 aptamers in HEL 92.1.7 or KG-1 cells by confocal microscopy, 1×10⁵ cells were plated. The cells were then gently washed with 1×PBS and re-suspended in 200 μl of binding buffer followed by the treatment with 50 μl of 250 nM and 25 nM FAM-labelled PD-1 aptamer and a non-specific aptamer (as negative control) for 30 min. After incubation, the labelled cells were washed three times with 1×PBS and fixed with ice-cold 4% paraformaldehyde for 5 min, and then attached to microscopic slides by Cytospin at 600k for 6 min. In case of PD-1 antibody labelling, cells were incubated with PD-1 primary antibody followed by secondary antibody. The slides were then air-dried for 5 min then mounted with mounting media with DAPI for nuclear staining (anti-fade reagent Invitrogen). The slides were then optically analyzed by using Axio Confocal Microscopy.

Binding selectivity test of specific PD-1 DNA aptamers to different types of cancer cells. Binding selectivity test provides an indirect measure for the rate of capture in different cancer cells models expressing PD-1 and selected PD-1 DNA aptamers. Different models of cancer cell lines (1×10⁵) were incubated with 10 μl of MyOne beads coated with specific PD-1 aptamer in the binding buffer for 30 min at a final volume of 200 μl. After incubation, the cancer cells were then washed 5×with 200 μl of washing buffer and then re-suspended in 200 μl of binding buffer. The number of captured cancer cells were counted using a hemocytometer under a bright field microscopy, whereas exclusion of MyOne beads would serve as control. The capture rate was calculated as follows:

$$capture rate ( \% ) = \frac{captured cancer cells}{total counted cancer cells from control tests} \times 100 \% \qquad ( 1 )$$

Reverse Phase Protein array (RPPA) to detect PD-1 expression levels using PD-1 DNA aptamer in different cancer models. Using specific PD-1 DNA aptamer in the replacement of PD-1 antibody in RPPA would be ideal choice for assessing PD-1 protein expression and post translational modifications in different cancer models. PD-1 DNA aptamers would serve as ideal PD-1 biomarker detection agent.

PD-L1 inhibitor screening ELISA assay to test the ability of specific anti-PD-1 aptamers or antibody to block the PD-1/PD-L1 interaction. The ability of PD-1 specific aptamers to block the PD-1/PD-L1 interaction was evaluated using a PD-L1 inhibitor screening ELISA assay. 96-well plate was coated with human PD-L1 protein and incubated with specific PD-1 aptamers or the standard antibody for positive control, and non-specific aptamer as negative control. Then biotinylated human PD-1 was added to the bound hPD-L1 followed by streptavidin-HRP. The color development was observed using TMB or other colorimetric HRP substrate. Finally, the ability of the PD-1 aptamer to inhibit PD-1: PD-L1 binding will be determined by OD measurement.

In vitro Functional Assay

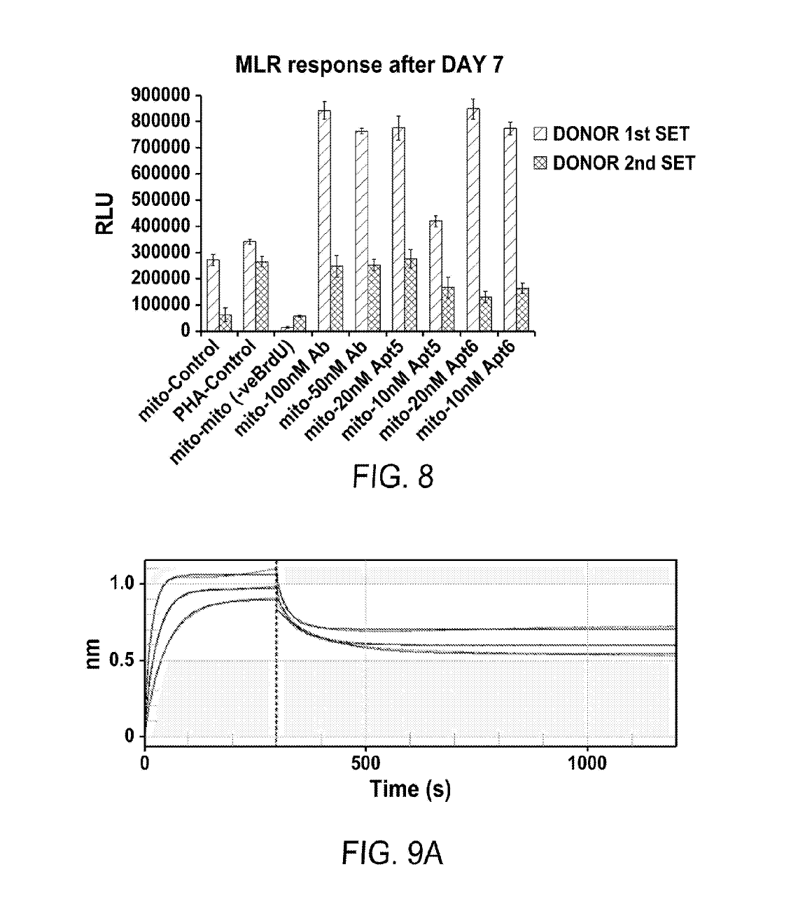

Mixed lymphocyte reaction. The ability of PD-1 specific aptamers to promote T-cell activation was evaluated using Allogeneic mixed lymphocyte reaction. Allogeneic MLR is a functional assay which measures the proliferative response of lymphocytes from one donor (the responder) to lymphocytes from another donor (the stimulator). PBMCs from both the responder and stimulator donor were isolated from the blood using Ficoll-Paque density gradient centrifugation. Stimulator PBMCs were incubated with 500 U/ml of interleukin-4 (IL-4) and 250 U/ml GM-CSF (Biolegend) in vitro for 7 days. After 7 days of incubation stimulator cells were treated with 50 μg/ml of mitomycin C (Sigma Aldrich) which binds to DNA rendering the cells non proliferative. One set of stimulator cells were treated with 2 μg/ml of PHA-M (Phytohemaagglutinin) required for a T-cell proliferative response as a positive control (Sigma Aldrich). Responder cells that are left untreated and were able to proliferate when stimulated. One set of responder cells were treated with 50 μg/ml of mitomycin C for negative BrdU control. Responder cells were either incubated with 20 ng/ml and 10 ng/ml PD-1 specific aptamers or non-specific aptamers for 24 hrs. Then the viable stimulator cells (150,000) were co-cultured with responder cells (100,000) according to different test conditions and incubated for 5 days. On day 6, bromodeoxyuridine (BrdU) is added to the wells to be incorporated in place of thymidine in the DNA of proliferating cells (BrdU Cell proliferation Chemiluminescent Assay, Cellsignal). After 24 hrs, the cells are analyzed for BrdU incorporation by a plate based-luminometer to measure Relative Light Units (RLU). Comparisons of the stimulated test cells with control, non-stimulated responder cells yields a stimulation index which can be used to compare proliferation in the various cell treatment combinations. Separate set of plate with the same set of conditions without BrdU was analyzed for viable cells by the quantitation of ATP present, an indicator of metabolically active cells by CellTiter-Glo Luminescent Cell viability assay (Promega). As per the data a robust MLR response is observed with 50 and 100 ng of antibody treatment and a comparable response is also observed with PD-1 specific aptamers (FIG. 8).

Suppression assay with regulatory T cells. CD4⁺CD25⁺ regulatory T cells (Tregs) and CD4⁺CD25⁻ responder T cells were purified from PBMCs (EasySep™ Human CD4+CD127lowCD25+ Regulatory T cell isolation Kit (Stemcell Technologies). Allogeneic mixed lymphocyte reaction assay were performed by co-culturing Tregs (5×10⁴) with responder T cells 1×10⁵ and 2×10⁴ stimulated dendritic cells (DCs) with either 20 ng/ml or 10 ng/ml of PD-1 specific aptamer or control non-specific aptamer. IFN-γ production was assessed in supernatants, and cells were labeled with (BrdU) in order to be incorporated in place of thymidine for additional 18 hrs for proliferation analysis.

Ex vivo cytokine-release assay. Heparinized blood (500 μl) from healthy donors were incubated with PD-1 specific aptamer or control non-specific DNA aptamers in 48-well plates at 37° C. and 5% CO₂ for 24 hrs. Cytokine responses (IL-2, IL-4, IL-6, IL-10, IFN-γ and TNF-α) were measured in plasma using the Cytometric Bead Array (Biolegend) and analyzed by FACSArray Bioanalyzer (BD Biosciences).

PEGylated Specific PD-1 Aptamers are not Cytotoxic and does not Trigger TLR9-Innate Immune Signaling

PEGylation of DNA aptamers. Specific and non-specific PD-1 DNA aptamers with a 3′ hexylamine modifier were incubated with a 10-fold excess of a 40 kDa mPEG-succinimidyl glutarate ester (P values for all the assays were calculated using Student's t-test or analysis of variance where appropriate. GraphPad PRISM software was used to plot all data, perform statistical analysis, and fit binding curves using a one site binding model.

Aptamer-induced TLR9 immune responses. PEGylated PD-1 specific and non-specific control aptamers (40 μg, 1.3 nmol) or a TLR9 CpG ODN (ODN 1826, Invivogen, CA) were administered i.p. into naïve C57BL/6 mice (n=3). Mice were sacrificed 3 hours later and serum levels of TNF-α and IL-6 were quantified using commercial ELISA sets (BD Biosciences). For in vitro studies, human macrophage cell line U-937 (ATCC) and mouse macrophage cell line RAW 264.7 (ATCC) were treated with 3 μmol/l CpG ODN 1585 or PD-1 specific aptamer or control non-specific aptamer for 3 hrs. TLR9 induction was observed by one-step real-time PCR reaction from the RNA isolated by RNeasy Mini Kit (Qiagen, Vaencia, Calif.) from the above treatments.

Statistics. P values for all the assays were calculated using Student's t-test or analysis of variance where appropriate. Graph Pad PRISM software was used to plot all data, perform statistical analysis, and fit binding curves using a one site binding model.

With such highly specific PD-1 DNA aptamers it is now possible to use this for biomarker screening process for detection of PD-1 proteins in clinical patient samples as well as to treat patients with many types of cancers including leukemia and many other PD-1 immune response related chronic diseases.

### Example 2

### PD1 Aptamer and Protein Binding Affinity

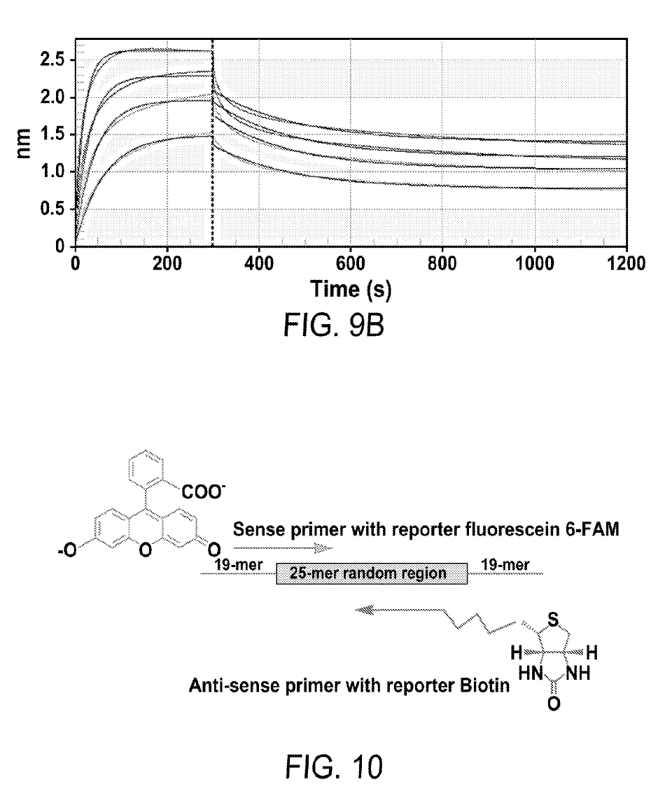

FIGS. 9A and 9B and Tables 3A and 3B below show aptamer fpf2 (10-150-373) binding to PD1 (FIG. 9A) and PD1 lysate (FIG. 9B).

TABLE 3A

| Conc. (nM) | Response | KD (M) |
|---|---|---|
| 6.25 | 1.0996 | 9.90E−09 |
| 3.13 | 0.9833 | 4.17E−09 |
| 1.56 | 0.9062 | 1.34E−09 |

TABLE 3B

| Conc. (nM) | Response | KD (M) |
|---|---|---|
| 6.25 | 2.6209 | 7.27E−10 |
| 3.13 | 2.3478 | 6.91E−10 |
| 1.5 | 2.0397 | 5.55E−10 |
| 0.5 | 1.525 | 2.86E−10 |

### Example 3

### Development and Characterization of PD1-Specific DNA Aptamers

Tumors have an extraordinary ability to escape immune response by modulating receptor-ligand interaction that regulate immune checkpoints. Programmed cell death protein (PD-1), a cell surface protein expressed on T cells is one such immune checkpoint receptor when bound to its ligands-PDL1 or PDL2 transmits an inhibitory signal. Such modulation often leads to inhibition of T-cell activation and subsequent escape of tumors from immune surveillance. Recently, several FDA approved therapeutic antibodies target PD-1/PD-L1 axis to enhance immune response against cancer. Aptamers are synthetic ligands composed of short, single-stranded oligonucleotides ranging from approximately 15 to 100 bases in length with defined 3-dimensional conformation and are analogous to the antibodies that can recognize and bind to their targets with high affinity. Also, the nucleic acid component has several advantages over the protein counterparts—such as ease of production under less stringent conditions, long shelf life and low cost. In this example, PD-1 specific aptamers consisting of highly repeating conserved regions (Anti-PD1-Apt) were assessed for target validation using leukemic cell lysates (cell lines and primary patient samples) and were found to bind to the PD-1 in its native state. Selected Anti-PD1-Aptamers were able to specifically pull down the PD-1 protein from the lysates mimicking anti-PD-1 antibody. The specific interaction of the Anti-PD1-Apt was also demonstrated by flow cytometry and fluorescent microscopy. Anti-PD1-Apt was able to bind to PD-1 with Kd of ˜500 picomolar affinity as assessed by Bio-Layer Interferometry. Furthermore, Anti-PD1-Apt biological activity was confirmed and characterized using a PD-1/PD-L1 cell-based assay using PD-1/NFAT-reporter Jurkat cells. Several fold induction was observed for NFAT luciferase reporter activity (Relative Luciferase Units) in PD-1/NFAT reporter-Jurkat cells co-cultured with HEK293 cells overexpressing PD-L1 and TCR activator in the presence of Anti-PD1-Apt compared to control. These data demonstrate robust Anti-PD1 blockade in Mixed Lymphocyte Reaction (MLR) along with induction of Th1 cytokines Interferon-gamma and IL-2 from different donor sets of PBMCs.

### Materials and Methods

Cell Culture: HEL 92.1.7, THP-1, Jurkat-clone E6-1 and HEK293 Cell lines were obtained from ATCC.HEL 92.1.7 THP-1 and Jurkat-clone E6-1 cell lines was cultured in RPMI1640 medium supplemented with 10% fetal bovine serum (FBS)and 100 IU/mL penicillin-streptomycin (as per ATCC instructions). HEK293 cell line was cultured in DMEM Hi-Glucose supplemented with 10% fetal bovine serum (FBS)and 100 IU/mL penicillin-streptomycin (as per ATCC instructions). KG-1 cell line was obtained from the DSMZ (Braunschweig, Germany). KG-1 cell line was cultured in IMDM supplemented with 20% fetal bovine serum (FBS)and 100 IU/mL penicillin-streptomycin (as per DSMZ instructions). All experiments with cell lines were performed within 6 months after thawing or obtaining from the ATCC and DSMZ. Cell lines authentication was performed by the ATCC and DSMZ. The DSMZ utilizes short tandem repeat (STR) profiling for characterization and authentication of cell lines. JURKAT recombinant cell line expressing PD-1/NFAT-reporter was obtained from BPS Bioscience. PD-1/NFAT Reporter/Jurkat T cells were maintained in RPMI1640 media supplemented with 10% fetal bovine serum (FBS), 100 IU/mL penicillin-streptomycin, 1 mg/ml Geneticin and 200 μg/ml of Hygromycin B to keep selection pressure on the cells. All cell lines were maintained at 37° C. with 5% CO₂ and 95% air and tested for mycoplasma using a MycoAlert mycoplasma detection kit (Lonza Rockland, Rockland, Me.) as described by the manufacturer. All experiments with the above-mentioned cell lines were conducted when cells were 70-80% confluent.

PBMCs and Primary AML blasts: Peripheral Blood Mononuclear cells (PBMCs) and Primary AML samples were obtained with informed consent in accordance with the Declaration of Helsinki. Peripheral blood or bone marrow aspirate samples were collected and separated for mononuclear cells by Ficoll-Hypaque density gradient centrifugation. Banked, delinked, and de-identified donor peripheral blood mononuclear cells that were procured, but not used for engraftment in the recipients, were purified before utilization in the cell viability assay and immunoblot analysis (Devaraj S G 2016).

Antibodies and Purified PD-1, PD-L1 proteins: Anti-PD-1 and anti-Actin antibodies were obtained from Sigma Aldrich (St. Louis, Mo.). Anti-PD-L1 antibody was obtained from ThermoFisher Scientific. Recombinant Human full length PDCD1 protein produced in human 293 cells (HEK293) and PD-L1 protein produced in human 293 cells (HEK293) were obtained from ACROBiosystems.

Quantification of Proteins, Western Analyses: Cells were subjected to lysis with ice-cold lysis buffer, and protein concentration was determined by using the BCA Assay Kit (Thermo Scientific, Waltham, Mass.). Equal amounts of cell lysates were used by SDS-PAGE for protein analysis or utilization for immunoprecipitation. Protein samples were separated on 10% polyacrylamide gels and then electro-transferred onto polyvinylidene fluoride membranes (PVDF) (EMD Millipore, Billerica, Mass.). PVDF membranes were blocked with blocking buffer (LI-COR), rinsed and incubated with primary antibodies against PD-1 and PD-L1. After overnight incubation at 4° C., membranes were washed and incubated with their corresponding Near-Infrared fluorescent secondary antibody (LI-COR). Immunoblots were scanned by an ODYSSEY CLx (LI-COR) for quantification of protein expression. Protein (3-actin (Sigma-Aldrich, St Louis, Mo.) was used as a loading control.

Immuno-precipitation Analyses : 100 μl of 1:1 mixture of protein A and protein G magnetic beads were washed three times with PBS-T and were used to pre-clear the total cell lysates protein of 500 μg from each HEL (ATCC) and KG-1 (DSMZ) cell lines and then discarded. Same amount of similarly prepared beads were incubated with 10 μg of anti-PD-1 antibody at 4° C. for 2 hrs. After the beads were washed with 3 times PBS-T buffer, pre-clear lysates corresponding to total protein of 500 μg from each HEL (ATCC) and KG-1 (DSMZ) cell lines were incubated overnight at 4° C. with antibody coated beads to capture PD-1 on to the surface. Such beads with captured PD-1 were separated from the supernatant and washed 3 times with PBS-T buffer. A small aliquot of such beads were subjected to immuno-blot analysis by SDS-polyacrylamide gel electrophoresis (PAGE) to confirm the presence of captured PD-1 and rest of them were used for SELEX rounds (Tuerk C 1990) as described below.

Isolation and Preparation of PD-1 Protein for SELEX: Endogenous PD1 and PD-L1 protein expression levels were compared in four distinct human cell lines (HEL 92.1.7, THP-1, Jurkat-clone E6-1 and KG-1) by immunoblot analysis after 6 and 24 hrs of interferon treatment. Based on the expression levels of PD-1 and PD-L1 after 24 hrs, two cell lines HEL 92.1.7 (ATCC) and KG-1 (DSMZ) expressing both PD-1 cell surface receptor and its ligand PD-L1 were selected for further isolation of PD1 individually by immunoprecipitation.

100 μl of 1:1 mixture of protein A and protein G magnetic beads were washed three times with PBS-T and were used to pre-clear the total cell lysates protein of 500 μg from each HEL (ATCC) and KG-1 (DSMZ) cell lines and then discarded. Same amount of similarly prepared beads were incubated with 10 μg of anti-PD-1 antibody at 4° C. for 2 hrs. After the beads were washed with 3 times PBS-T buffer, pre-clear lysates corresponding to total protein of 500 μg from each HEL (ATCC) and KG-1 (DSMZ) cell lines were incubated overnight at 4° C. with antibody coated beads to capture PD-1 on to the surface. Such beads with captured PD-1 were separated from the supernatant and washed 3 times with PBS-T buffer. A small aliquot of such beads were subjected to immuno-blot analysis by SDS-polyacrylamide gel electrophoresis (PAGE) to confirm the presence of captured PD-1 and rest of them were used for SELEX rounds (Tuerk C 1990) as described below.

SELEX Procedure, Next Generation Sequencing and Data Analysis: One Nano mole of the purchased library was suspended in 50 ml of PBS pH 7.4 containing 2 mM MgCl2 and heated to 95° C. for 10 minutes in water bath and then allowed to cool slowly at room temperature. After cooling to room temperature, magnetic beads coated with anti-PD1 antibody were added to the library mix and incubated for 30 minutes at room temperature to remove those aptamers that have high affinity towards magnetic beads as well as anti-PD1 antibody. The beads were removed by magnetic separation and the supernatant containing the reduced library was now incubated with magnetic beads having captured PD-1 and incubated for 1 hour at room temperature. After incubation, the beads were separated and thoroughly washed three times (10 ml each time) with PBS pH 7.4 containing 2 mM MgCl₂. The washed beads were used as template in PCR amplification with primers AMLIB3 and AMLIB4 (see library design). PCR conditions were 95° C. for 3 min initial denaturation; [95° C. for 45 seconds, 54° C. for 45 seconds and 72° C. for 45 seconds]×20 cycles; followed by 72° C. for 5 minutes final extension, FIG. 2. Single strand DNA for next iterative round of selection was prepared by capturing the biotinylated strand (arising out of biotinylated primer) on the streptavidin magnetic beads followed by strand separation in presence of 0.1M NaOH. NaOH was removed by 10K centricon spin filtration from the single stranded DNA prep. Seven rounds of Iterative selection were performed in a similar fashion as described above each time with freshly prepared PD-1 magnetic beads. At each round, an aliquot of selected pool of PCR product was stored for PCR amplification using primers with additional “tag” sequences to allow later identification of their source following next generation sequencing. Such analysis is highly useful to track convergence of binding sequence with the iterative rounds of selection. Next-generation sequencing was performed on a single Ion-torrent 314 chip (SeqWright) after pooling PCR products in equal proportions from all the seven rounds and two different lines each having tracking tags (unique barcode) at 5′ and 3′ positions.

Analysis of the NGS data was be performed using Galaxy project sequence analysis tools and FastAptamer software suite (Khalid K Alam 2015). The software has manual tools to sort and align the sequencing data into clusters and tally each sequence as a function of DNA tag allowing us to identify best binding sequences to PD-1. Multiple sequence comparison and dendrograms were further generated using clustal X (Thompson J D 1997).

Aptamer synthesis: Aptamers were synthesized on Expedite 8909 Oligo Synthesizer (Midland Oligos, Midland, Tex.) using standard phosphoramidite chemistry (Caruthers M H 1987). Aptamers were deprotected in concentrated ammonium hydroxide overnight at room temperature, they were vacuum dried overnight, and they were purified by reverse phase chromatography over a Hamilton PRP-1 column on an AKTA 10 purifier (General Electric), by loading using a 100 mM triethylamine acetate buffer (pH 8.4) and eluting with increasing acetonitrile concentrations. Aptamer concentrations were determined using extinction coefficients estimated by OligoCalc (WA 2007).

Validation of Binding Specificity of Selected DNA aptamers to PD-1: To determine the ability of selected aptamers to recognize endogenous PD-1, PD-1 pull-down assays were performed from total cell lysate of HEL 92.1.7 (ATCC) using six best represented biotinylated aptamers. The PD-1 protein levels from the pull-down assays were comparable to immunoprecipitated protein using PD1-specific antibodies.

Two selected 15-120-298 and 10-150-373 aptamers were further validated for PD-1 binding by flow cytometry and microscopy. Briefly, the fluorescently labeled aptamer were incubated with HEL cells expressing PD-1 and thoroughly washed to remove the unbound aptamer. The cells were then analyzed by flow cytometry for mean fluorescent intensity. Also, the cells were examined by fluorescence microscopy.

Secondary Structures of PD-1 Specific Aptamers: mfold algorithm was used to predict the secondary structures of selected PD-1 DNA aptamers. For all the aptamers, the optimal and suboptimal structures had a minimum free energy in range of −1.5 to −4.0 kcal/mole suggesting structures with low stability. These aptamers may adapt a G-quadruplex structures that may contributed to the specificity/affinity to recognize PD-1 (Bochman M L 2012).

Filter binding assay: Binding affinity of PD-1-DNA aptamer to PD-1 protein was determined by dual membrane filter binding assay. PD-1-DNA aptamer with biotin-TEG modification at 5′ end for streptavidin-HRP chemiluminescent detection was synthesized and purified as described above. One μl of 50 nM PD-1 DNA aptamer was incubated with varying concentration of PD-1 protein (1000-0 nM, serial half-dilutions) in 3.5 μl of 10 mM Tris-HCl pH 7.4 (TE buffer) for 30 minutes at room temperature. After incubation, the volume of each reaction was made up to 30 μl with TE buffer and filtered through a 96-well dot-blot apparatus (Bio-Rad) with nitrocellulose layered on top of the nylon membrane. The top nitrocellulose retains protein-DNA complex and bottom nylon membrane would trap free DNA. The membranes were washed with 100 μL of TE three times to wash away any unbound DNA from the nitrocellulose membrane down to the nylon. Following washes, both the membranes were UV cross-linked to immobilize the retained DNA and were processed for chemiluminescent detection using the Pierce biotinylated nucleic acid detection kit according to the manufacturer's instructions. The chemiluminescent signals were detected and imaged on a Fluorochem imager (Alpha Innotech). Image analysis and quantification of spot intensities were conducted usingImageJ software. Data from nylon membrane was used to calculate the fraction bound and binding curve generated using graph pad prism software assuming a single site binding (Oehler S 1999).

Biolayer interferometry binding studies: The binding kinetics of purified PD-1 protein and total cell lysate containing PD-1 protein complex with PD-1 DNA aptamers were characterized with an Octet RED96 (ForteBio) Bio-Layer Interferometry at 25° C. in the assay buffer (10 mM phosphate pH 7.4, 137 mM NaCl, 2.7 mM KCl, 0.1% bovine serum albumin (BSA) and 0.01% Tween-20). Specific PD-1 5′ biotinylated DNA aptamer (Midland Oligos) were prepared in the concentration range of (10 nM and 100 nM). The biotinylated aptamers were immobilized on to streptavidin biosensor tips, followed by the measurement of BLI signals upon the association of different concentrations of purified PD-1 protein and PD-1 protein from total cell lysate (1 μM, 0.5 μM, 0.25 μM, 0.125 μM, 0.063 μM, 0.0312 μM, 0.0156 μM, and 0 μM) and the subsequent dissociation of PD-1 protein into blank assay buffer. The dissociation constant (KD) of each DNA were obtained by fitting the full titration range of the PD-1 binding data with a 1:1 model of association and dissociation functions ForteBio and (Kelly Hew and 2016). Octet Red analysis software (ForteBio) was used to analyze the sensorgram data.

Filter binding assay for determination of PD-1 DNA aptamer function as antibody: Function of PD-1 DNA aptamer mimicking the role of PD-1 antibody was determined by PVDF membrane filter binding assay. PD-1-DNA aptamer with biotin-TEG modification at 5′end for Near-Infrared fluorescent streptavidin secondary Ab (LI-COR) detection was synthesized and purified as described above. One μl of different concentration of total cell lysate from HEL and KG-1 cell lines containing PD-1 protein (100 μg-0.78 μg, serial half-dilutions) and purified PD-1 protein (10 μg-0.078 μg, serial half-dilutions) was filtered through a 96-well dot-blot apparatus (Bio-Rad) onto the PVDF membrane. The membranes were washed with 100 μL of TE three times to wash away any unbound protein from the PVDF membrane. Following washes, the membranes were UV cross-linked to immobilize the retained protein. Then the PD-1 protein bound PVDF membranes were blocked with blocking buffer (LI-COR), rinsed and incubated with primary PD-1 DNA aptamer and antibody against PD-1. After 1 hr incubation at room temperature (RT), membranes were washed and incubated with their corresponding Near-Infrared fluorescent secondary antibody (LI-COR). Immunoblots were scanned by an ODYSSEY CLx (LI-COR) for quantification of protein expression (Oehler S 1999).

Affinity competition between PD-1 DNA aptamer and antibody by flow cytometry: To determine the competition between PD-1 DNA aptamer and antibody HEL 92.1.7 and KG-1 cell lines (1×10⁵ cells for each) respectively, were incubated with 100 nM and 10 nM of carboxyfluorescein (FAM) labelled PD-1 specific aptamers and 500 nM and 50 nM of PD-1 antibody for 15 min in 50 μl of binding buffer. Cells were then washed 5-6 times with 100 μl of wash buffer and then re-suspended in 50 μl of binding buffer for analysis via flow cytometry and 1×10⁴ cells were counted for analysis. The cells without aptamers treatment serve as the background control.

Affinity competition between PD-1 DNA aptamer and antibody by fluorescence microscopy: To determine the competition between PD-1 DNA aptamer and antibody HEL 92.1.7 and KG-1 cell lines (1×10⁵ cells for each) respectively, were incubated with 100 nM and 10 nM of carboxyfluorescein (FAM) labelled PD-1 specific aptamers and 500 nM and 50 nM of PD-1 antibody for 15 min in 50 μl of binding buffer. Cells were then washed 5-6 times with 100 μl of wash buffer and then re-suspended in 50 μl of binding buffer for analysis via flow cytometry and 1×10⁴ cells were counted for analysis. The cells without aptamers treatment serve as the background control.

Mixed Lymphocyte Reaction: The ability of PD-1 specific aptamers to promote T-cell activation was evaluated using Allogeneic mixed lymphocyte reaction. Allogeneic MLR is a functional assay which measures the proliferative response of lymphocytes from one donor (the responder) to lymphocytes from another donor (the stimulator). PBMCs from both the responder and stimulator donor were isolated from the blood using Ficoll-Hypaque density gradient centrifugation. Stimulator PBMCs were incubated with 500 U/ml of interleukin-4 (IL-4) and 250 U/ml GM-CSF (Biolegend) in vitro for 7 days. After 7 days of incubation stimulator cells were treated with 50 μg/ml of mitomycin C (Sigma Aldrich) which binds to DNA rendering the cells non-proliferative. One set of stimulator cells were treated with 2 μg/ml of PHA-M (Phytohemaagglutinin) required for a T-cell proliferative response as a positive control (Sigma Aldrich). Responder cells that are left untreated and were able to proliferate when stimulated. One set of responder cells were treated with 50 μg/ml of mitomycin C. for negative BrdU control. Responder cells were either incubated with 20 nM/ml and 10 nM/ml PD-1 specific aptamers or non-specific aptamers and also with 100 nM/ml of PD-1 antibody for positive control for 24 hrs. Then the viable stimulator cells (150,000) were co-cultured with responder cells (100,000) according to different test conditions and incubated for 5 days. On day 6, bromodeoxyuridine (BrdU) is added to the wells to be incorporated in place of thymidine in the DNA of proliferating cells (BrdU Cell Proliferation Chemiluminescent Assay, Cellsignal). After 24 hrs, the cells are analyzed for BrdU incorporation by a plate based-luminometer to measure Relative Light Units (RLU). Comparisons of the stimulated test cells with control, non-stimulated responder cells yield a stimulation index which can be used to compare proliferation in the various cell treatment combinations. Separate set of plate with the same set of conditions without BrdU was analyzed for viable cells by the quantitation of ATP present, an indicator of metabolically active cells by CellTiter-Glo Luminescent Cell viability assay (Promega). As per the data a robust MLR response is observed with 50 and 100 ng of antibody treatment and a comparable response is also observed with PD-1 specific aptamers (K. V Bromelow 2001) and Xeno Diagnostics LLC.

ELISA analysis with PD-1 DNA Aptamer and Antibody: The supernatant collected from the MLR assay day 1, day 3 and day 7 were subjected to ELISA analysis for IL-2, IL-4 and IFN-γ expression levels using (BD Biochem ELISA kit) according to manufacturer's instruction.

Screening and Characterization of Biological activity of PD-1 and its ligand interaction using cell-based assay in the context of anti PD-1 DNA aptamer: To test the validity of anti PD-1 DNA aptamer PD-1 DNA-aptamer, HEK293 cell line were transfected with either TCR activator or Human PD-L1 TCR activator and after 24 hrs of transfection pre-incubate with anti-PD1 aptamer or antibody. Then PD-1/NFAT Reporter-Jurkat cells were added to TCR and PD-L1 TCR transfected HEK293 in the presence of anti PD-1 DNA aptamer or antibody with different concentrations. Relative luciferase or luminescence units were measured using a luminometer. The fold induction of PD-1/NFAT luciferase reporter expression was calculated by background-subtracted luminescence of stimulated well/average background-subtracted luminescence of unstimulated control wells (Mei Cong 2015).

Aptamer stability in human serum: Each PD-1 DNA-aptamer (2 μM) dissolved in 1×PBS was incubated in 96% or 80% human serum from (Sigma Aldrich) for periods ranging from 1 h to 7 days at 37° C. At each time point indicated aliquots of 5 μL of the serum (20 pmol DNA) was withdrawn and incubated for 1 h at 37° C. with 5 μL of proteinase K solution (600 mAU/mL) to remove serum proteins that interfere with electrophoretic migration. Following proteinase K treatment, 18 μL of denaturing gel loading buffer was added to each sample, and the samples were then stored at −80° C. Serum samples without DNA aptamer would serve as negative control. Samples from each time point were separated by electrophoresis on 15% denaturing polyacrylamide gels. The gels were stained with ethidium bromide and visualized by ultraviolet light exposure(Ken-ichiro Matsunaga 2015).

Immunohistochemistry using aptamers to detect PD-1: Formalin fixed paraffin embedded tissue slides were provided by IHC World. The tissue sections were cut on an rotary microtome at 5 um thickness and mounted on charged slides and baked overnight at 50 C oven. All staining procedures are performed at room temperature.

PD-1 Antibody IHC Staining: The slides were deparaffinized and rehydrated to water. Antigen retrieval were performed using steam and proteinase K digestion methods. For steam method, the slides were steamed in IHC-Tek Epitope Retrieval Solution (IW-1100) for 35 minutes and then cooling for 20 minutes. For proteinase K method, the slides were incubated in IHC-Tek Proteinase K Solution (IW-1101) for 20 minutes in 37 C oven and then cooling for 10 minutes. After antigen retrieval, the slides were allowed to cool at room temperature for 20 minutes prior to the next step. Then the slides were washed in three changes of PBS for 5 minutes each and the blocked with 3% H2O2. After washing in three changes of PBS, the slides were incubated in PD-1 Antibody (Abcam, Cat#ab52587) diluted 1:50 and 1:100 with antibody diluent for 1 hour at room temperature. The slides were then washed three times in PBS and incubated with Horse Anti-Mouse Secondary Antibody (Vector Lab) for 30 minutes. Then slides were washed with PBS for three times, 5 minutes each and incubated with HRP-Streptavidin (Jackson Immunoresearch, 1:500) for 30 minutes. Then incubate with DAB Chromogen Substrate Solution (IHC World, Cat#IW-1600, 0.05% DAB) for 5-10 minutes and then wash with PBS and counterstained with Mayer's hematoxylin.

PD-1 Aptamer IHC Staining: The slides were deparaffinized and rehydrated to water. Antigen retrieval were performed using steam and proteinase K digestion methods. For steam method, the slides were steamed in IHC-Tek Epitope Retrieval Solution (IW-1100) for 35 minutes and then cooling for 20 minutes. For proteinase K method, the slides were incubated in IHC-Tek Proteinase K Solution (IW-1101) for 20 minutes in 37 C oven and then cooling for 10 minutes. After antigen retrieval, the slides were allowed to cool at room temperature for 20 minutes prior to the next step. Then the slides were washed in three changes of PBS for 5 minutes each and the blocked with 3% H2O2. After washing in three changes of PBS, the slides were incubated with biotinylated PD-1 Aptamer diluted 1:500 nM and 1:1000 nM with aptamer diluent for 1 hour at room temperature. Then slides were washed with PBS for three times, 5 minutes each and incubated with HRP-Streptavidin (Jackson Immunoresearch, 1:500) for 30 minutes. Then incubate with DAB Chromogen Substrate Solution (IHC World, Cat#IW-1600, 0.05% DAB) for 5-10 minutes and then wash with PBS and counterstained with Mayer's hematoxylin. The stained slides were examined with regular microscope. Representative photographs were taken.

Target killing assay to detect the function of PD-1 aptamer: The ability of PD-1 specific aptamers to promote immune cell activation (T cell subpopulation of peripheral blood mononuclear cells (PBMCs) was evaluated by quantifying the killing of HEL-92-1 tumor cells by measuring the fluorescence intensity area of Annexin V-positive cells by Incucyte.

This functional assay measures the mean fluorescence intensity area generated due to the Annexin V-positive labeling. PBMCs (effector) cells and HEL.92.1 (target) cells were used for this assay. PBMCs from donor were isolated from the blood using Ficoll-Hypaque density gradient centrifugation. Stimulator PBMCs were incubated with 500 U/ml of interleukin-4 (IL-4) and 250 U/ml GM-CSF (Biolegend) in vitro for 5 days. Target cells were either incubated with either 20 nM/ml PD-1 specific aptamers or non-specific aptamers or 100 nM/ml of PD-1 specific aptamers were co-cultured in the presence of viable effector cells either 20:1 or 10:1 ratio. Then Annexin V solution (Incucyte) was added to these co-cultured cells and allowed them to settle on a level surface for at least 30-60 min to allow even distribution. the cell plate was placed into the Incucyte ZOOM instrument and allow the plate to warm at 37oc for 10-20 min prior to the first scan. The scans were scheduled for every 2-3 hr for 24 hr repeats up to 5 days.

In vivo Experiments (Animal Models): All animal housing and handling procedures were in accordance with CrownBio IACUC protocol and has been conducted in accordance with Crown Biolnternational R&D Center Standard Operating Procedures (SOPs). Six-week-old NOD/SCID mice (Crown Bio) (n=3) were assigned into two groups. Overall average weight of the mice was 18-22 gm. All animal housing and experimental procedures were performed in accordance with institutional guidelines of Crown Bio. The pharmacokinetic study was performed in 6-week old NOD/SCID mice by injecting PD-1 DNA aptamer tagged with CY3 and PD-1 DNA aptamer tagged with CY3-P10. In the single-dose acute study (45 μM in 100 μl saline) intravenously once via the tail vein. 50 μl of blood is drawn as negative control before injecting PD-1 aptamer and (diluted to 1:10 and 1:100 in phosphate buffered saline (PBS). CY3-labelled PD-1 aptamer of (45 μM/100 μl saline) and CY3-P10-labelled PD-1 aptamer of (45 μM/100 μl saline) were injected in mice and 50 μl of blood is collected at 8 time points from 5 min-720 min. Then the plasma from these time points was diluted to 1:10 and 1:100 in PBS. CY3 fluorescence was measured using CY3 filters at excitation peak at 550 nm and emission peak at 570 nm. Pharmacokinetic parameters were obtained by fitting plasma concentration-time data to a two-compartment model using Phoenix®WinNonlin® version 6.3 (Certara USA, Inc., Princeton, N.J.) (Kang S A 2015).

Anti PD1 aptamer-p10 efficacy is comparable to Anti-PD-1 antibody Next, anti PD1 aptamer efficacy was analyzed for comparison to anti PD-1 antibody. A HEL92.1.7 model was used to engraft in humanized NSG. Study has 3 groups and 3 animals per group from a single cohort: Vehicle group, anti-PD-1 and anti-PD-1 aptamer. Humanized NSG mice are inoculated with 5,000,000 cells subcutaneously in the right rear flank. IV, BIW 3 doses of anti-PD-1 antibody and IV, BIW 5 doses of anti-PD-1 aptamer (total of 8 doses for this study) a day after randomization when average tumors are between 60-90 mm³ were implemented for this in vivo study. Animals are monitored for 4-6 weeks for survival. Animals are euthanized if tumor volume exceeds 2000 mm³ or the body weight loss is greater than 20%, as per IACUC protocol regulation.

Statistical Analysis: Binding curves calculations were performed using Graph Pad Prism 6.0 software (Graph Pad Software Inc., San Diego, Calif.).

### REFERENCES CITED

Bochman M L, P. K., Zakian V A. (2012). “DNA secondary structures: stability and function of G-quadruplex structures.” Nature Reviews Genetics 13(11): 770-780.
Caruthers M H, B. A., Beaucage S L, Dodds D R, Fisher E F, McBride L J, Matteucci M, Stabinsky Z, Tang J Y. (1987). “Chemical synthesis of deoxyoligonucleotides by the phosphoramidite method.” Methods Enzymology 154: 287-313.
Devaraj S G, F. W., Shah B, Qi J, Sun B, Iyer S P, Sharma S, Bradner J E, Bhalla K N. (2016). “HEXIM1 induction is mechanistically involved in mediating anti-AML activity of BET protein bromodomain antagonist.” Leukemia 30(2): 504-508.
K. V Bromelow, W. H., R. L Mendes, A. R Winkley, I. E Smith, M. E. R O'Brien, B. E Souberbielle (2001). “Whole blood assay for assessment of the mixed lymphocyte reaction.” Journal of Immunological Methods 247(1-2): 1-8.

Kang S A, T. B., Mann A P, Zheng W, Zhao L, Zhao Y D, Volk D E, Lokesh G L, Morris L, Gupta V, Razaq W, Rui H, Suh K S, Gorenstein D G, Tanaka T (2015). “Safety evaluation of intravenously administered mono-thioated aptamer against E-selectin in mice.” Toxicology and Applied Pharmacology 287(1): 86-92.

Kelly Hew, S. V., Daniel Sim, Tobias Cornvik, Par Nordlund and Sue-Li Dahlrotha and (2016). “Structure of the opening reading frame 49 protein encoded by Kaposi's sarcoma associated herpesvirus (KSHV).” Journal of Virology 01947-16.

Ken-ichiro Matsunaga, M. K., Charlotte Hanson, Michael Sanford, Howard A. Young, Ichiro Hirao (2015). “Architecture of high-affinity unnatural-base DNA aptamers toward pharmaceutical applications.” Nature Scientific Reports 5(18478): 1-7.
Khalid K Alam, J. L. C. a. D. H. B. (2015). “FASTAptamer: A Bioinformatic Toolkit for High-throughput Sequence Analysis of Combinatorial Selections.” Molecular Therapy Nucleic Acids 4(e230): 1-10.
Mei Cong, Z.-J. J. C., Natasha Karassina, Jamison Grailer, Jim Hartnett, Neal Cosby, Frank Fan (2015). “Novel bioassay to assess PD-1/PD-L1 therapeutic antibodies in development for immunotherapy.” Genetic Engineering News 35(10).
Oehler S, A. R., Barker A. (1999). “Is nitrocellulose filter binding really a universal assay for protein-DNA interactions?” Analytical Biochemistry 268(2): 330-336.
Thompson J D, G. T., Plewniak F, Jeanmougin F, Higgins D G. (1997). “The CLUSTAL_X windows interface: flexible strategies for multiple sequence alignment aided by quality analysis tools.” Nucleic Acid Research 25(24): 4876-4882.
Tuerk C, G. L. (1990). “Systematic evolution of ligands by exponential enrichment: RNA ligands to bacteriophage T4 DNA polymerase.” Science 249(4968): 505-510.
W A, K. (2007). “OligoCalc: an online oligonucleotide properties calculator.” Nucleic Acid Research 35: 43-46.

### RESULTS

PD-1 and PD-L1 Overexpression is Associated with Poor Survival in Cancer Patients

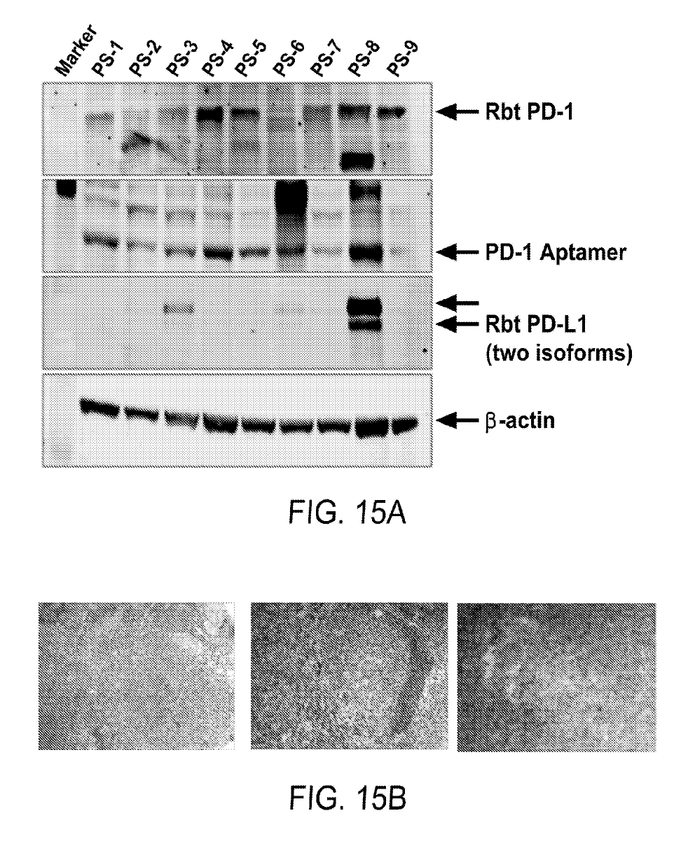

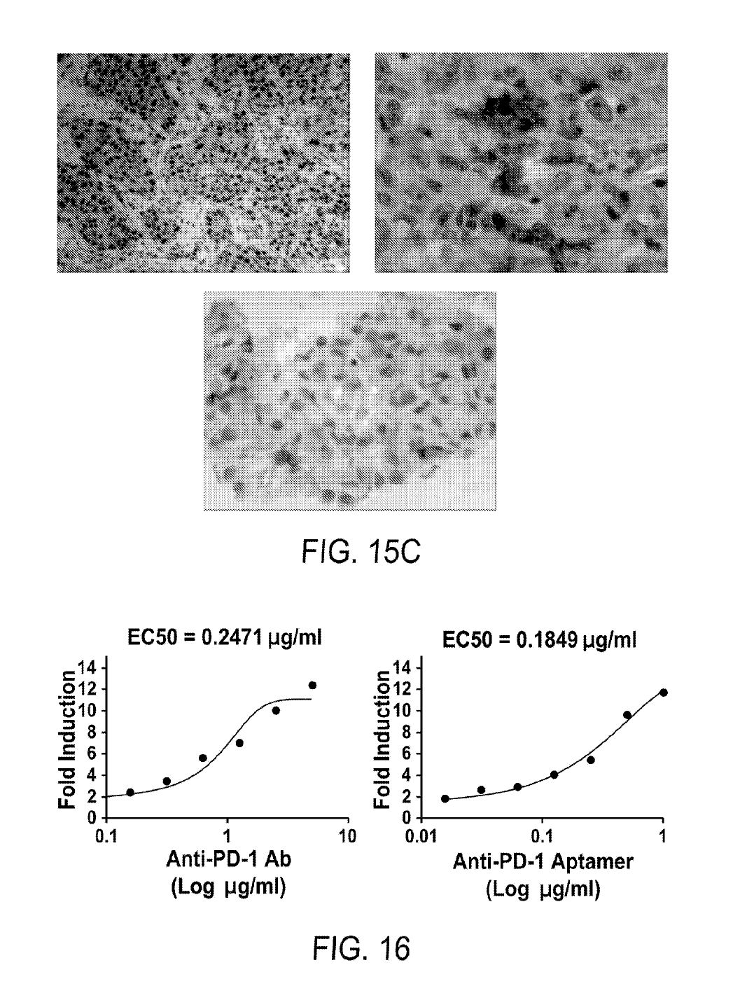

First, the hematological cancer (HC) dataset of The Cancer Genome Atlas (TCGA) database was analyzed to determine the association of immune-checkpoint PD-1 and its ligand PD-L1 expression with HC patient survival (FIG. 15A). TCGA data showed that over-expression of PD-1 and its ligand PD-L1 was correlated with tumor progression and with poor survival in HC patients (FIG. 15A). Furthermore, western analysis for levels of PD-1 and its ligand PD-L1 protein expression in a panel comprising four leukemic cell lines found that PD-1 and PD-L1 was highly expressed in most of the leukemic cell lines (FIG. 15A). Immuno-histochemical analysis of PD-1 and PD-L1 protein expression in tumor samples from patients with high-grade serious HC revealed high PD-1 and PD-L1 expression in these tumors (FIG. 15A, FIG. 15B and FIG. 15C). These findings point to the potential importance of PD-1 as a therapeutic target for HC patients.

Identification of Human Endogenous PD-1 Binding DNA Aptamers

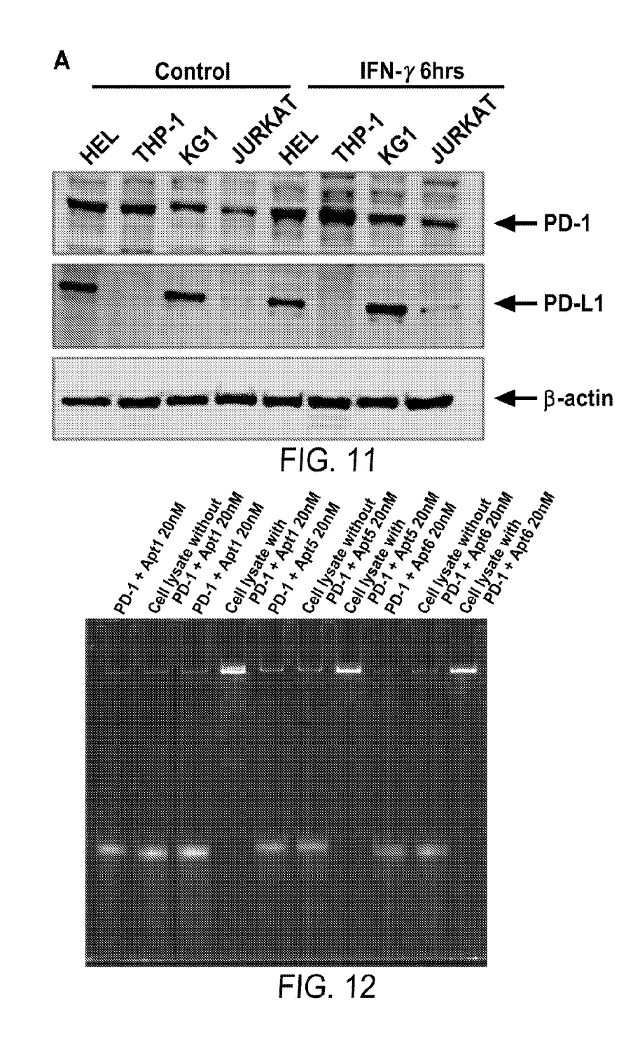

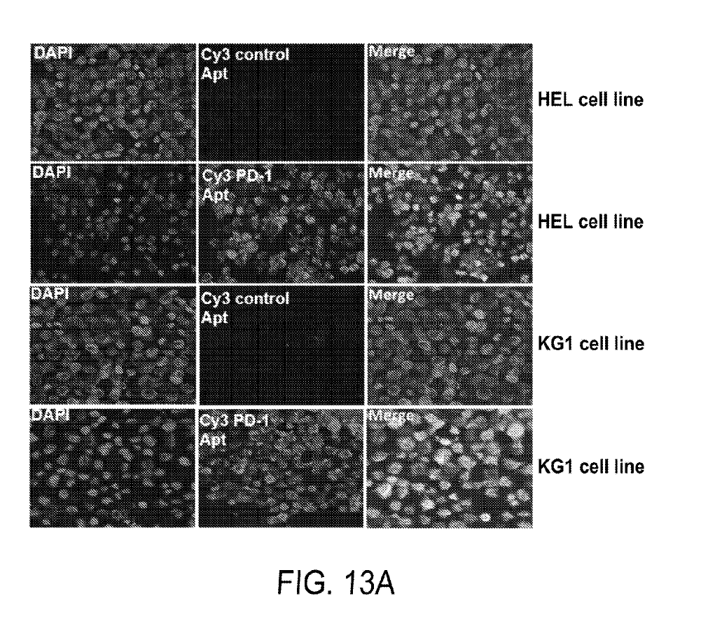

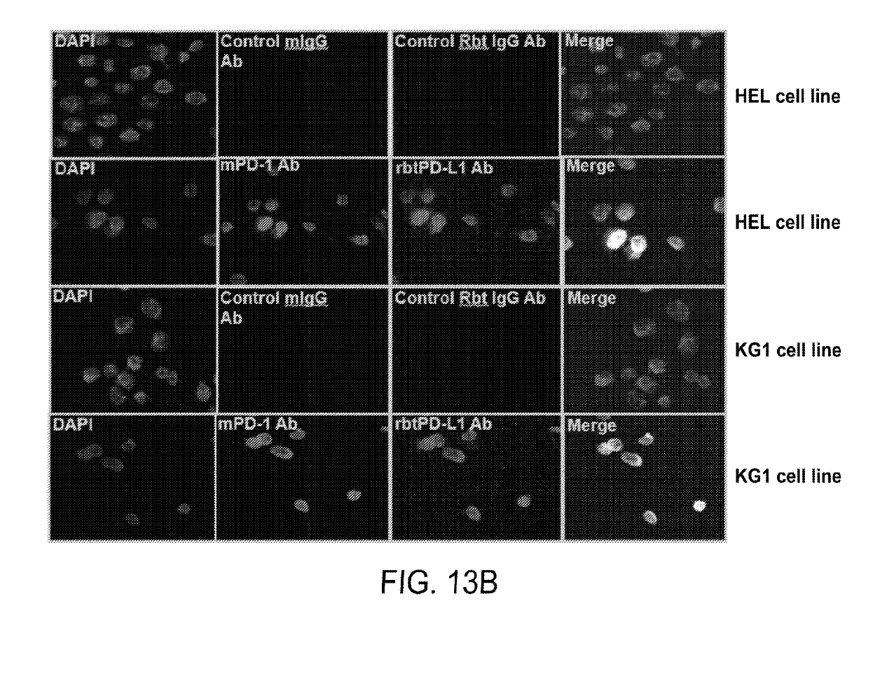

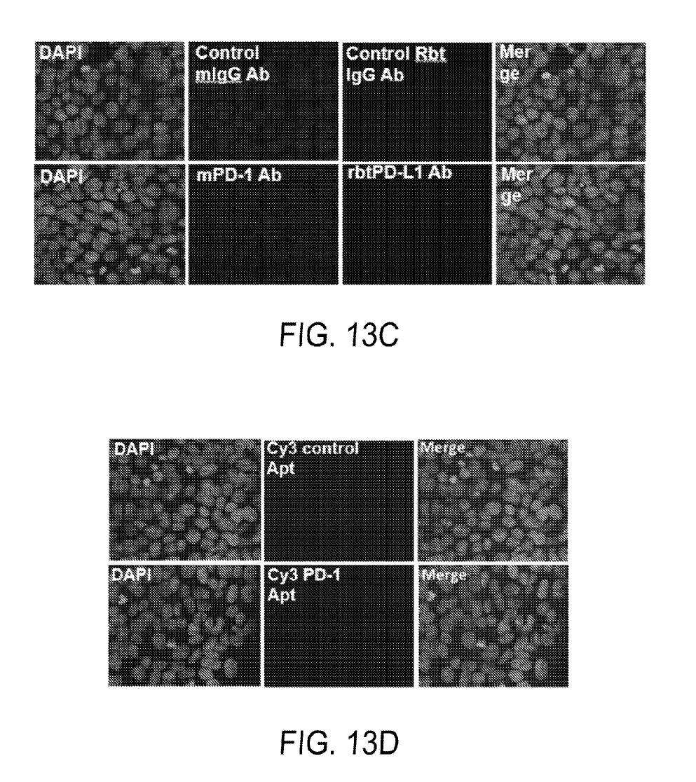

In vitro-based SELEX was performed to identify DNA aptamers which specifically recognize the endogenous human PD-1 (hPD-1) by screening a random DNA library against endogenously expressed immuno-precipitated PD-1 from leukemic cells lines HEL 92.1.7 and KG-1 using specific PD-1 antibody (FIG. 1, FIG. 2, FIG. 10, FIG. 11) and expression levels of PD-1 and PD-L1 were confirmed by confocal microscopy (FIG. 13A, FIG. 13B, FIG. 13C and FIG. 13D). Seven iterative rounds of enrichment were performed each time with freshly prepared immuno-precipitated native PD-1 and a counter selection step was performed by beads bound to anti PD-1 antibody to remove the aptamer sequences which are specific for streptavidin beads or the antibody from the selected pool in each iterative round. Next-generation sequencing (NGS) was performed on a single Ion-torrent 314 chip (SeqWright) after pooling PCR products in equal proportions from all the seven rounds and two different lines each having tracking tags (unique barcode) at 5′ and 3′ positions. NGS data revealed a significant enrichment of aptamer families harboring sequences with high sequence identity to a parental highly enriched sequence. Specifically

Clustal X multiple sequence analysis of converged sequences from rounds 6 and 7 revealed a highly repetitive element with “T and G”s which were observed in all present converged sequences (FIG. 3 and FIG. 4). Immuno-precipitation analysis with six such biotinylated versions of these aptamers immobilized onto magnetic streptavidin beads was used to immuno-precipitate hPD-1 from total cell extracts of HEL and KG-1 cell lines, confirming that these sequences were enriched from the bulk library during selection due to their ability to bind hPD-1 in its endogenous native complex (FIG. 5). The ability of anti PD-1 DNA aptamer to bind hPD-1 was confirmed by immunoprecipitation with HEL and KG-1 cytoplasmic and nuclear extracts. PD-1 expression was observed in the cytoplasmic extract comparable to PD-1 antibody rather than nuclear extract.

Anti-PD-1 DNA Aptamers are Stable

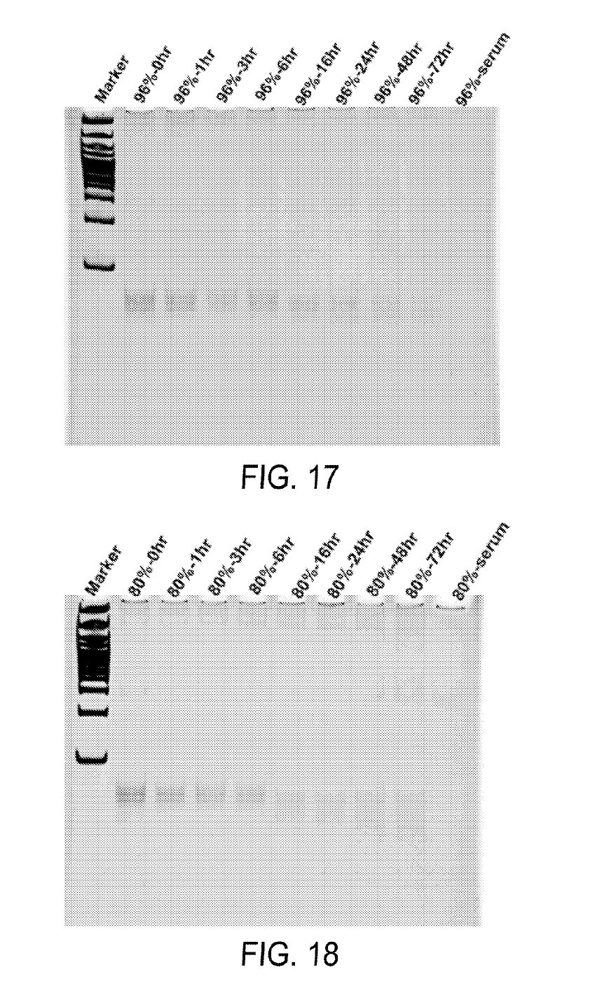

The highest enriched best represented biotinylated anti PD-1 DNA aptamer sequences were chosen for further evaluation. Structural predictions performed using M fold algorithm on selected aptamer sequences has revealed a complex hairpin-bulge folding state. The folded aptamer sequences with the optimal and suboptimal structures of minimum Gibbs free energy of −1.5 to −4.0 kcal/mol was observed (FIG. 7). All of the enriched DNA sequences contain a high prevalence of G-rich repetitive regions, hence it is possible that these aptamers adapt a G-quadruplex structures that may contribute to the specificity/affinity to recognize PD-1 (FIG. 7). The stability of the anti PD-1 DNA aptamer in medium containing either 96% or 80% human serum from 1 hr to 7 days was evaluated by denaturing polyacrylamide gel electrophoresis. These data clearly show that anti PD-1 DNA aptamer was stable for up to 72 hr in both 96% and 80% serum (FIG. 17 and FIG. 18).

Anti-PD-1 DNA Aptamers Bind with Nanomolar Affinity to Purified Overexpressed PD-1 Protein and Picomolar Affinity to Endogenous PD-1 Protein Present in Total Cell Extract

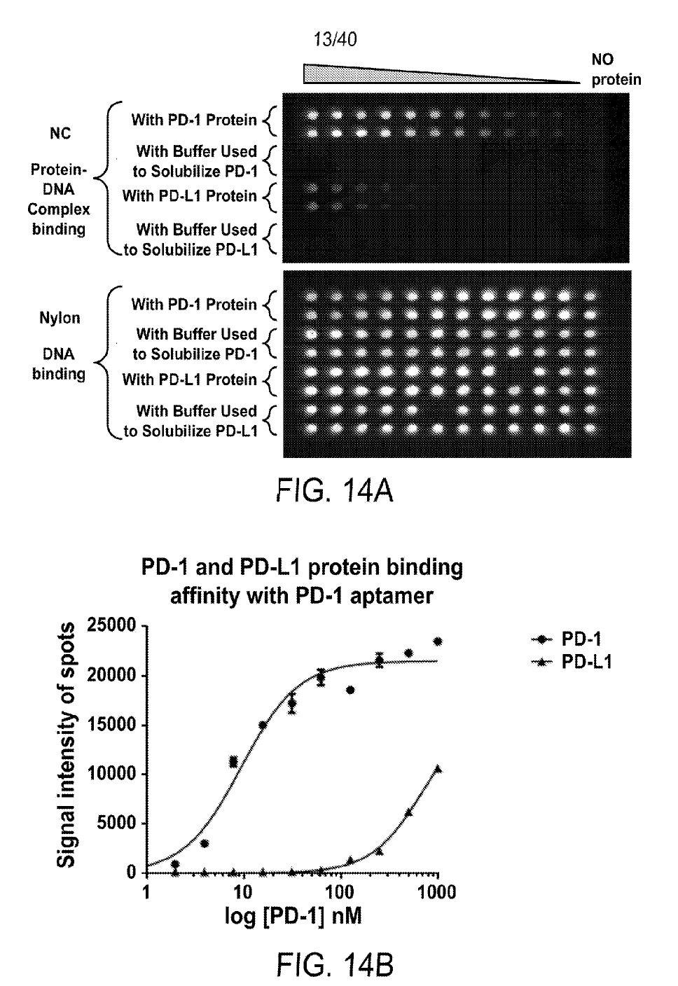

Biolayer interferometry was used to generate binding kinetics data with purified PD-1 protein and total cell lysate (HEL and KG-1 cell line) defined the dissociation constant of anti PD1-DNA aptamer as 5 nM and 500 pM (FIG. 9A and FIG. 9B). Dissociation constant were confirmed by nitrocellulose filter binding assay demonstrating that anti PD-1 DNA aptamer binds to total cell lysate of PD-1 expressing cell lines in picomolar concentration in its native conformation better than the purified over-expressed PD-1 protein (FIG. 14A and FIG. 14B). To evaluate the localization of this anti PD-1 DNA aptamer in cells, HEL and KG-1 cell lines were incubated for 30 min with fluorescence-labeled anti PD-1 DNA aptamer (250 nM). After fixing the cells, immunofluorescence was used to visualize the localization. Anti PD-1 DNA aptamer was localized at the cell surface, whereas negative control cell line not expressing PD-1 displayed no signal (FIG. 6B).

Competition Binding Assay of Anti-PD-1 DNA Aptamer Binding Sites Followed by Anti PD-1 Antibody.

To further validate whether anti PD-1 DNA aptamers and anti PD-1 antibody compete for the same PD-1 binding sites by flow cytometry, immunofluorescence and gel-shift assays were performed. To determine the competition between PD-1 DNA aptamer and antibody for PD-1 binding sites HEL 92.1.7 and KG-1 cell lines incubated with 100 nM and 10 nM of carboxyfluorescein (FAM) labeled PD-1 specific aptamers followed by 500 nM and 50 nM of PD-1 antibody for 15 min in 50 μl of binding buffer. Mean fluorescence intensity by flow cytometry revealed in the presence of antibody the aptamer binding affinity is down regulated suggesting both antibody and antibody share at least similar PD-1 binding sites (FIG. 6A). The competition for PD-1 binding sites was confirmed by fluorescence microscopy (FIG. 6B). Gel shift assay also revealed the similar affinity for PD-1 binding sites between antibody and aptamer (FIG. 12).

Biological Activity of Anti PD-1 DNA Aptamer in PD-1 and its Ligand PD-L1 Interaction Using in vitro Cell-Based Assay

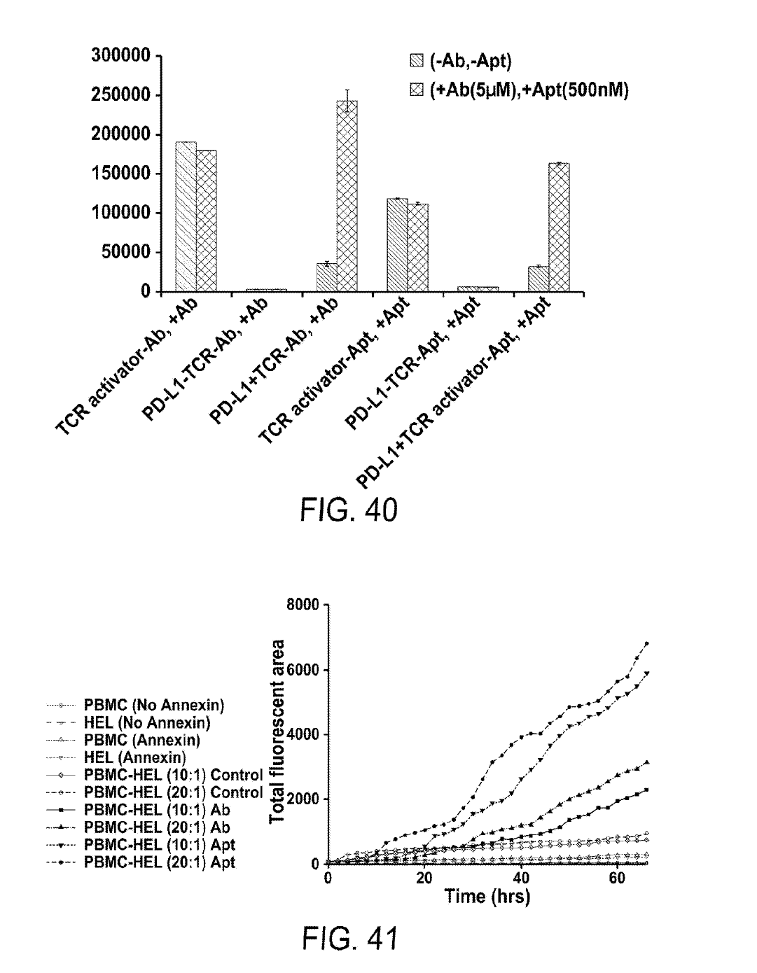

The functionality of anti PD-1 DNA aptamer was validated using PD-1: PD-L1 cell based assay. Jurkat T cells expressing PD-1/NFAT were used as effector cells whereas HEK293 cells over-expressing an engineered T cell receptor (TCR) along with PD-L1 are used as activator cells. Co-culturing of these two cells (effector+TCR activator) results in expression of the NFAT luciferase reporter whereas (effector+TCR activator+PD-L1) results in prevention of TCR activation and hence suppressing the NFAT-responsive luciferase activity. Specific inhibition relief was observed when treated with anti-PD-1 DNA aptamer in this combination (effector+TCR activator+PD-L1) and anti PD-1 neutralizing Ab were used as positive control (FIG. 40). The increase in relative luciferase units in the presence of anti PD-1 DNA aptamer suggesting the role of anti PD-1 DNA aptamer mimicking PD-1 neutralizing ab in blocking PD-1: PD-L1 interaction and promote T cell activation, resulting in reactivation of the NFAT responsive luciferase reporter. A dose response curve was also shown for the positive control anti-PD-1 neutralizing Ab (FIG. 16A) with an EC50 value of 0.2471 μg/ml and for anti-PD-1 DNA aptamer (FIG. 16B) with an EC50 value of 0.1849 μg/ml. These results show that anti PD-1 DNA aptamer promotes T cell activation in lower concentration than the antibody.

In vitro biological activity of anti PD-1 DNA aptamer by MLR assay

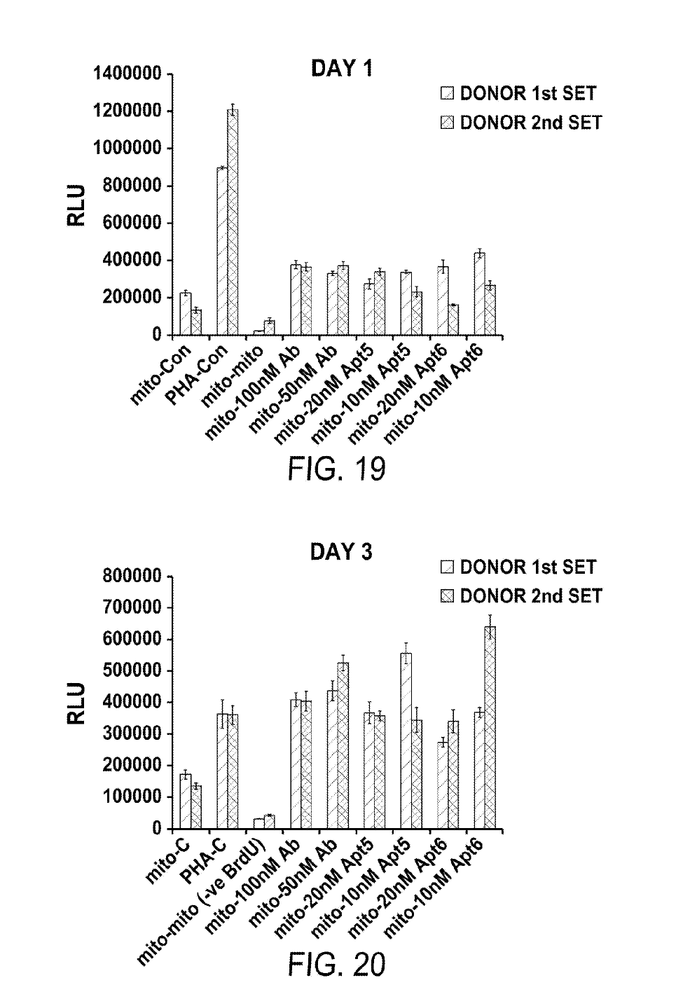

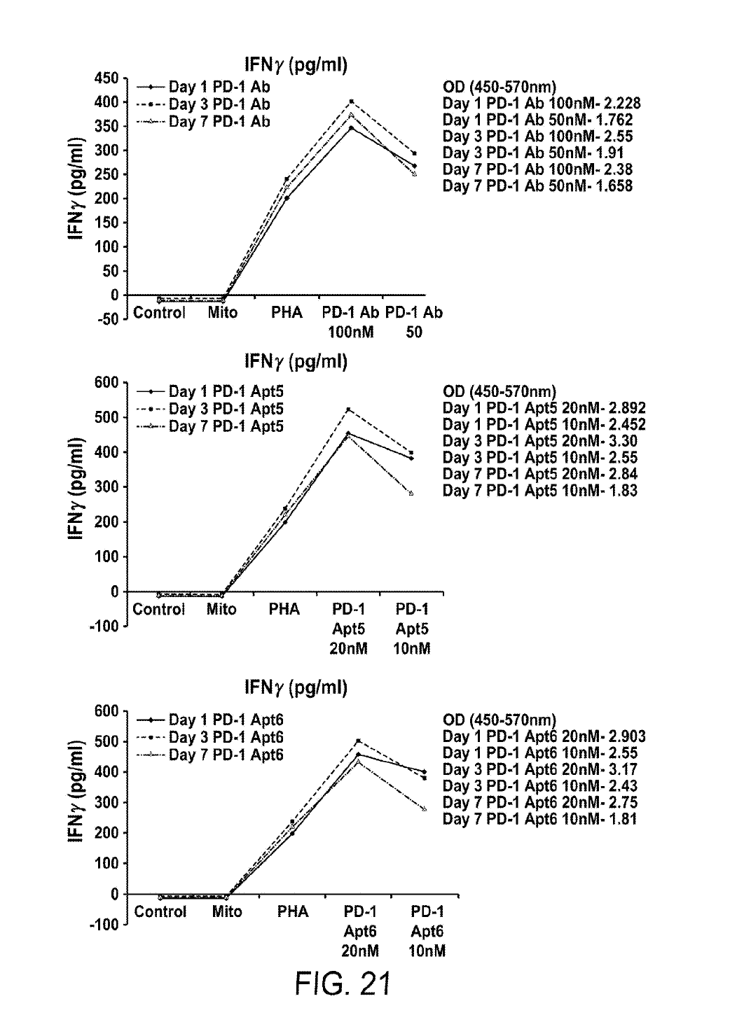

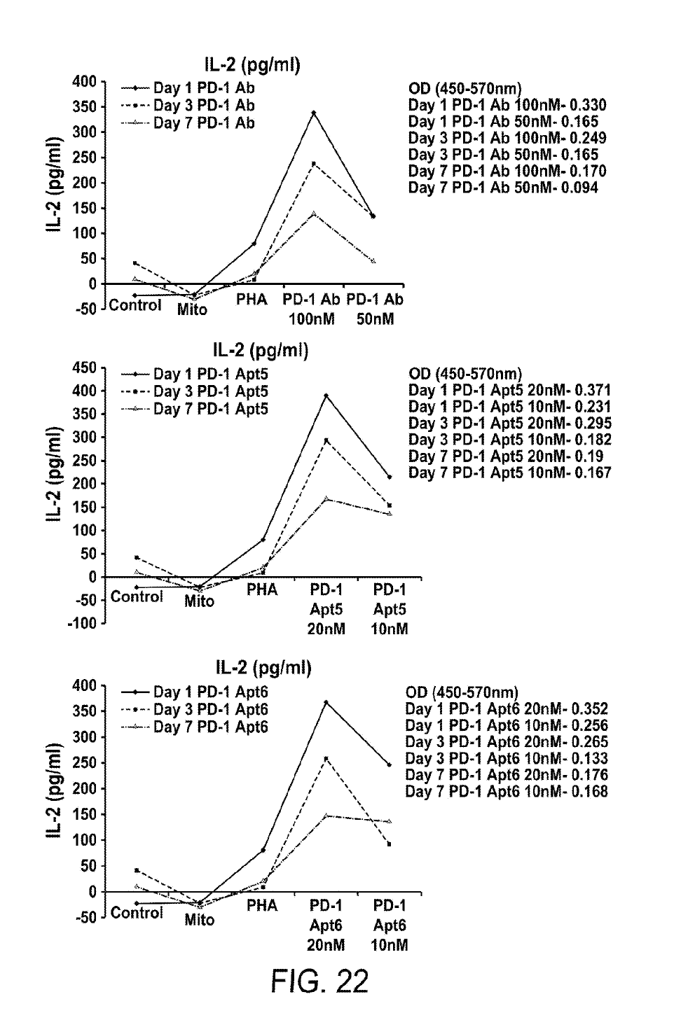

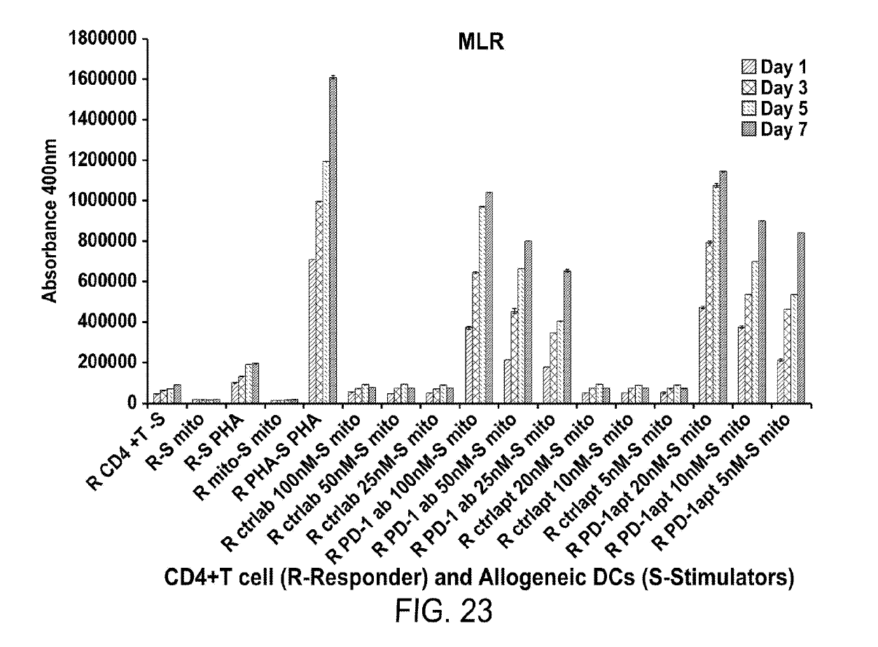

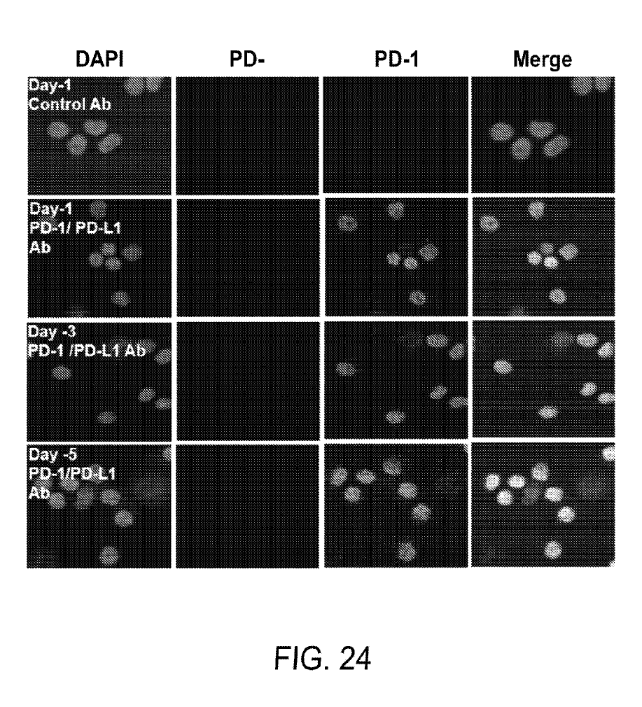

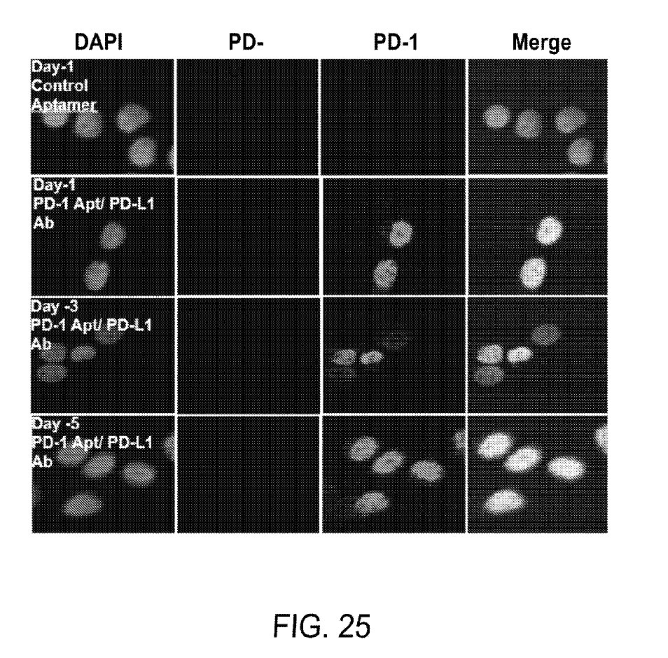

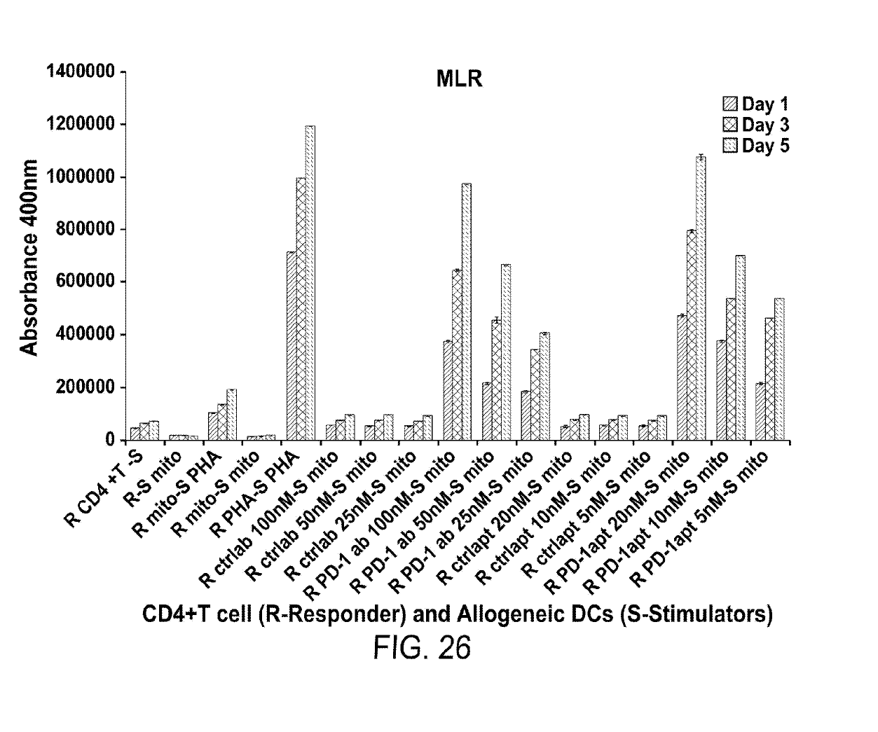

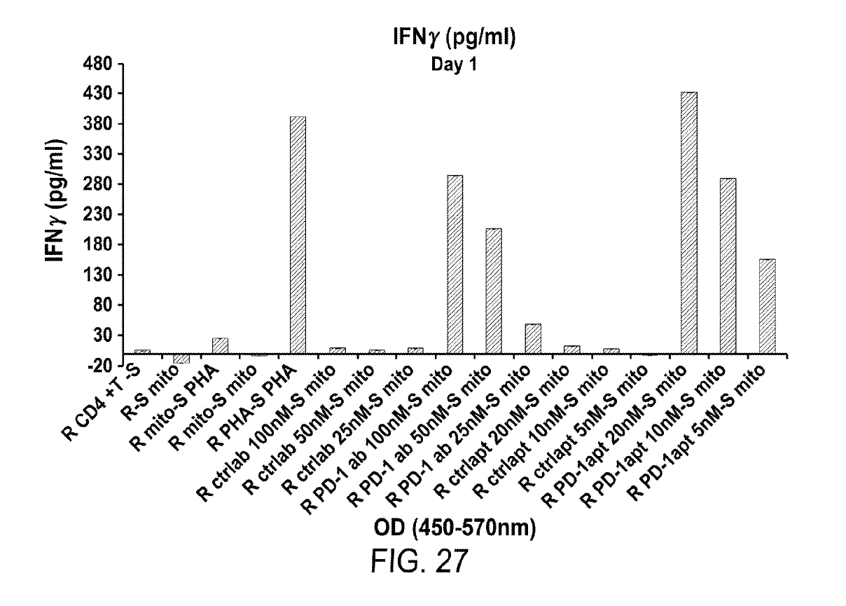

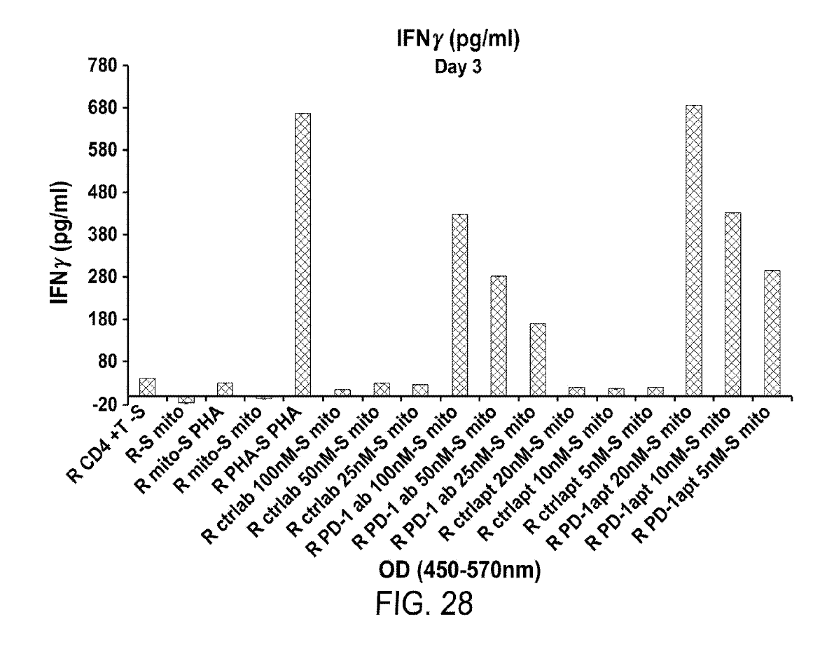

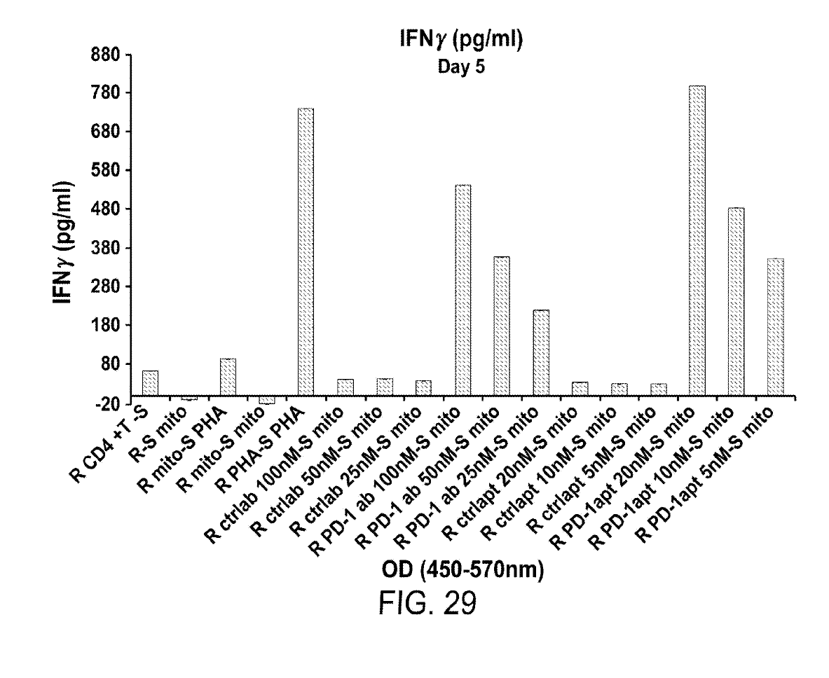

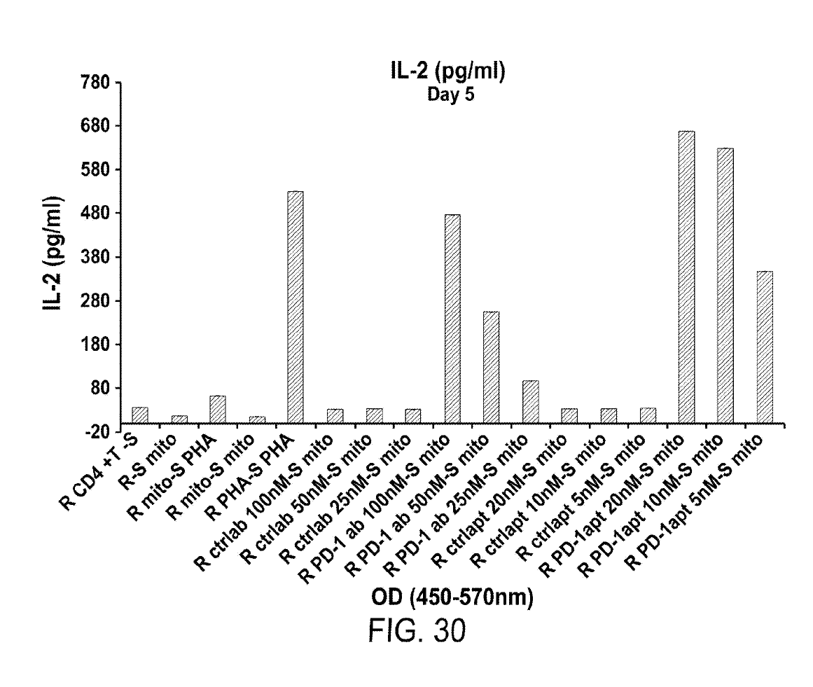

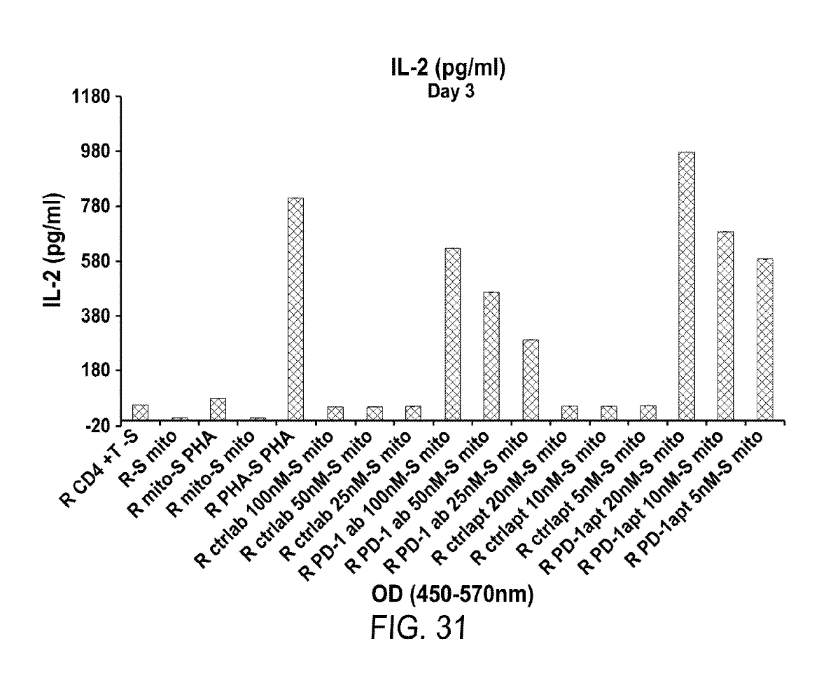

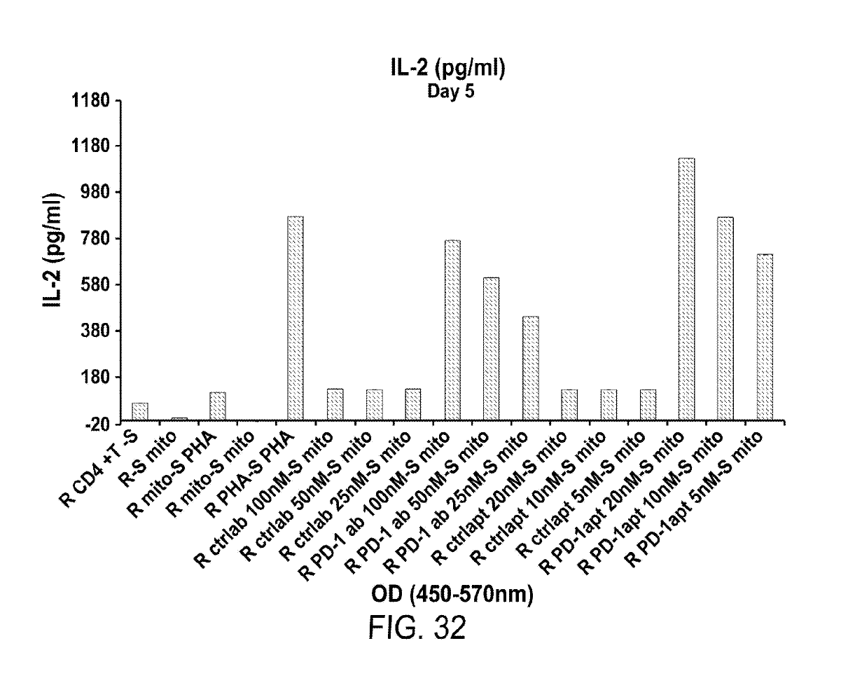

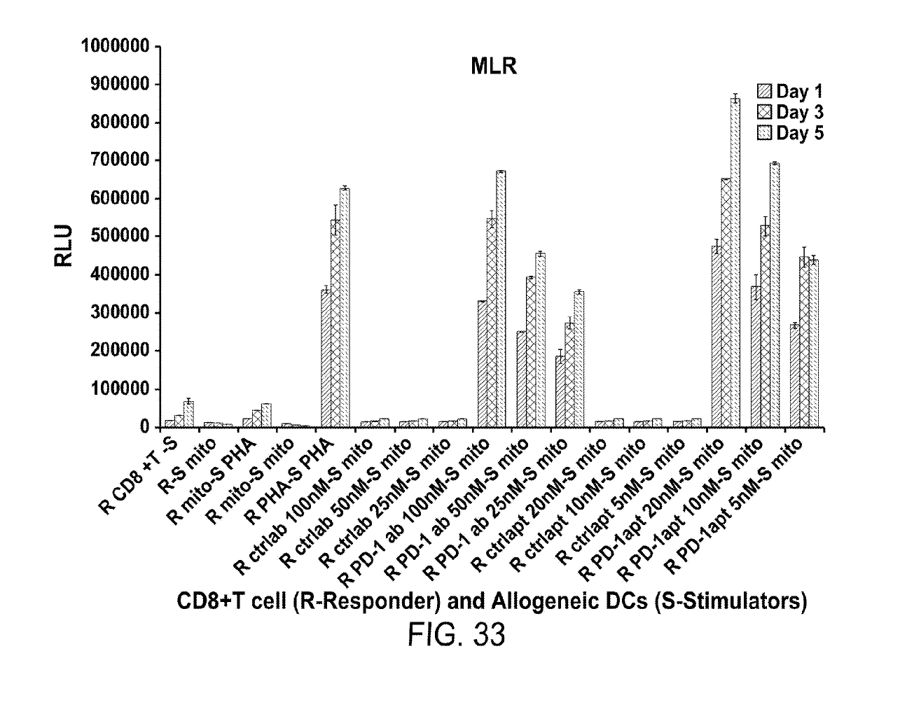

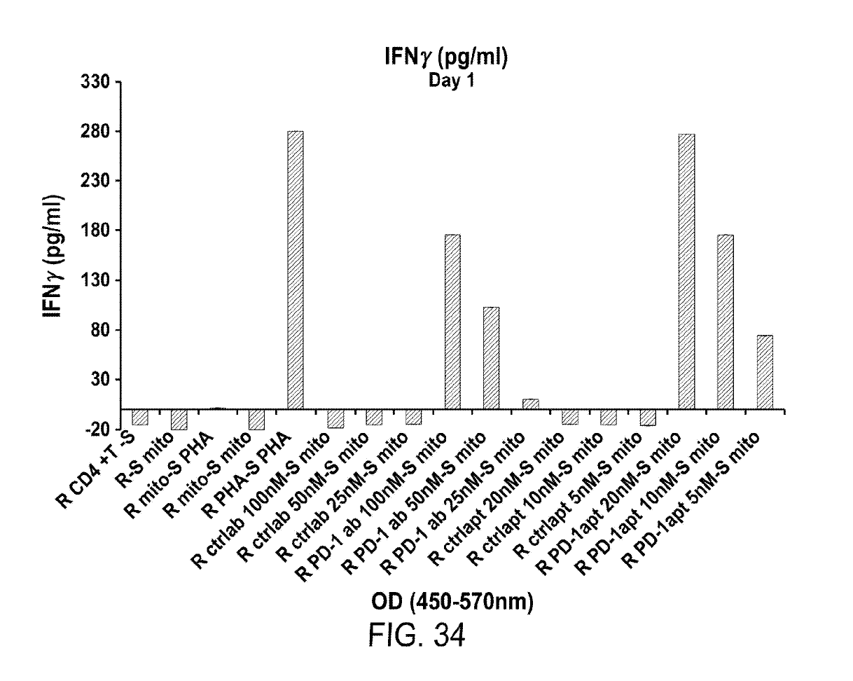

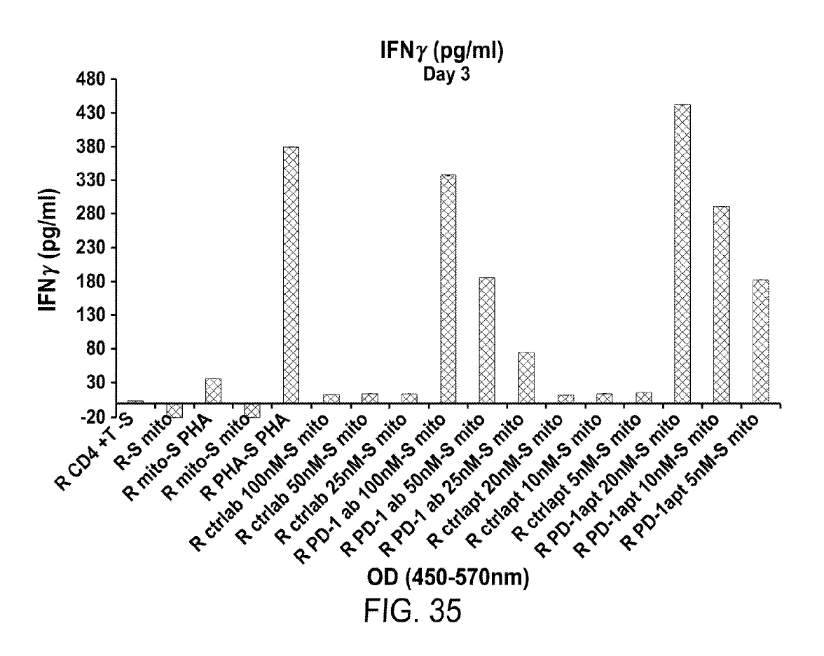

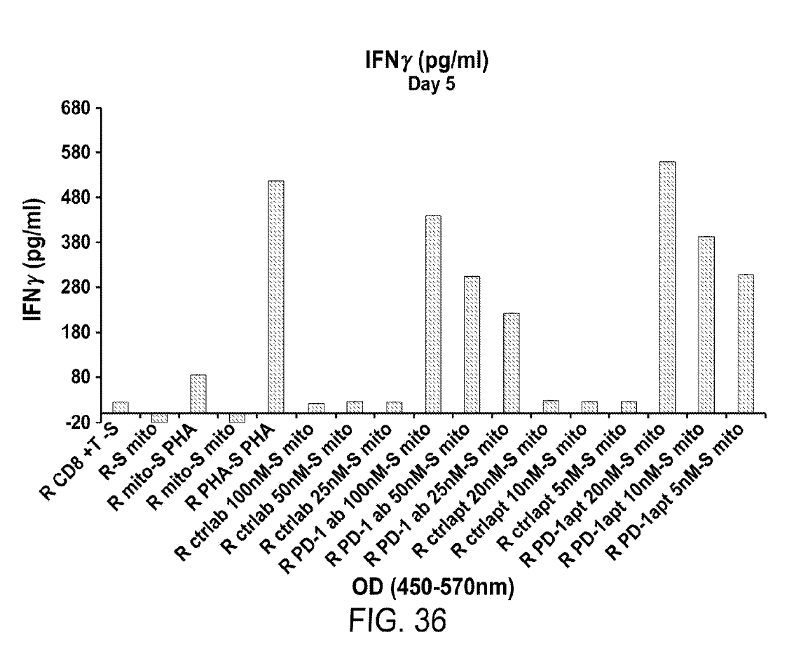

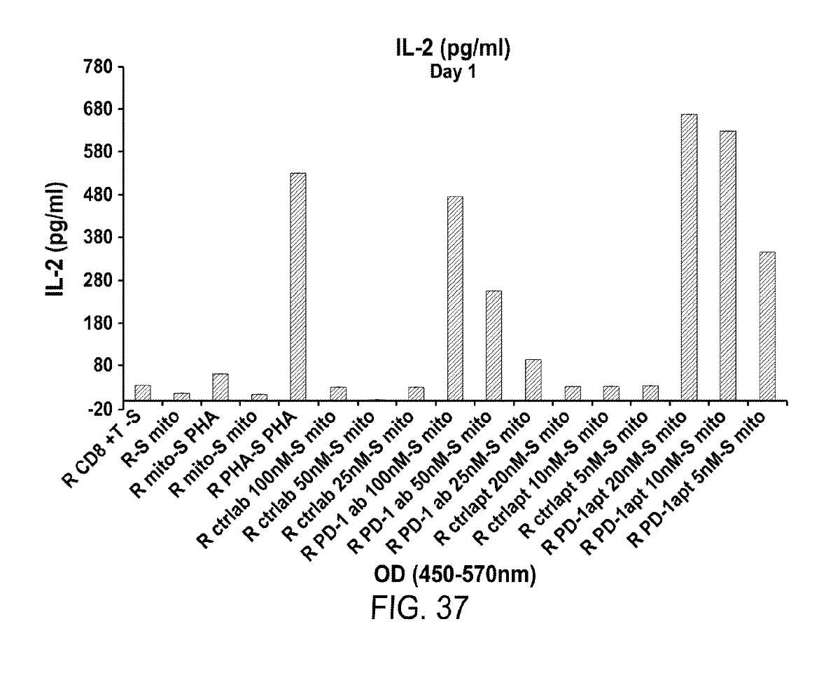

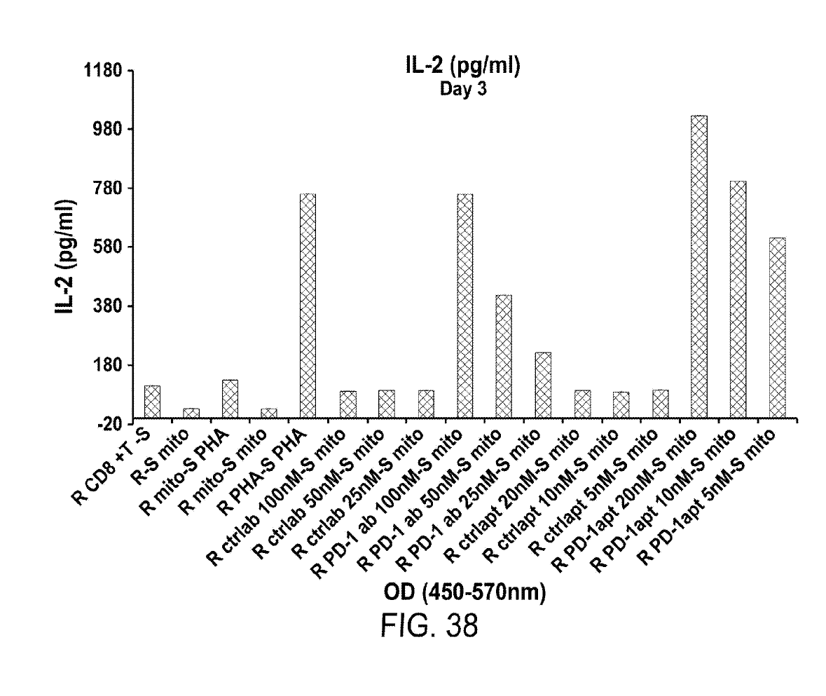

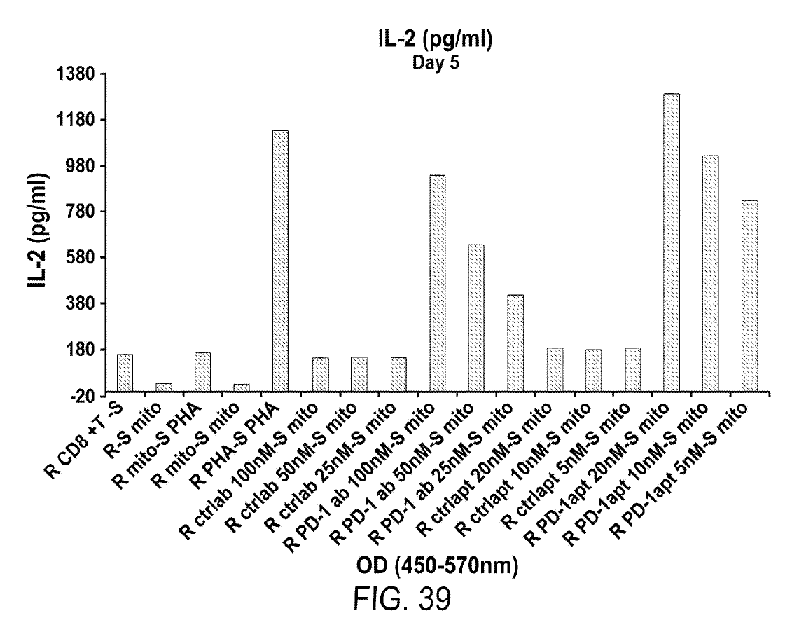

In order to assess the kinetics of the MLR response in the presence of anti PD-1 DNA aptamer to promote T-cell activation, proliferation assays were performed. Serial measurement of BrdU incorporation was demonstrated after 5 day stimulation. Maximal lymphocyte proliferation was seen around day 1 after 5 day stimulation in the presence of anti PD-1 DNA aptamer (concentration of 20 nM/ml and 10 nM/ml) (FIG. 8) and control PD-1 neutralizing ab (concentrations of 100 nM/ml and 50 nM/ml) (FIG. 8). A better proliferative response was observed in the presence of lower concentrations of anti PD-1 DNA aptamer (FIG. 19 and FIG. 20). The number of viable cells on the culture remained constant for the first 5 days of activation and in the presence of either anti PD-1 DNA aptamer or antibody proliferation started to peak around day 6 and slowly started declining at day 8. A similar pattern of proliferative response was observed for viability of the cells without BrdU incorporation by quantification of ATP presence by using cell titer CellTiter-Glo Luminescent Cell viability assay. A robust MLR response is observed in the presence of anti PD-1 DNA aptamer in comparable antibody treatment. Enhancement of IFN-y response (FIGS. 21A-21C) as well as IL-2 secretion (FIGS. 22A-22C) was also observed in the presence of anti PD-1 DNA aptamer comparable to the PD-1 antibody in the supernatants of the MLR assay samples by ELISA. Serial measurement of the concentration of IFN-γ response in the culture medium peaked at day 6 after 5 day stimulation and declining of IL-2 secretion can be seen around day 8 (FIGS. 21A-21C and FIGS. 22A-22C). Serial measurement of the concentration of IL-2 in the culture medium peaked at day 6 after 5 day stimulation and declining of IL-2 secretion can be seen around day 8 (FIGS. 22A-22C). Overall, addition of anti PD-1 DNA aptamer increased IL-2 secretion by a mean of ˜80 fold to 380 fold over control and IFN-γ response by a mean of ˜200 fold to 400 fold over control. PD-1 and PD-L1 expression in CD4+T cells was also examined by confocal microscopy (FIG. 24 and FIG. 25). A similar pattern of MLR response was observed with CD4+T cells (FIG. 23 and FIG. 26) and CD8+T cells (FIG. 33). An increase in the concentration of IFN-γ response was observed in the supernatants of CD4+T cells (FIG. 27, FIG. 28 and FIG. 29) as well as in the supernatants of CD8+T cells (FIG. 34, FIG. 35 and FIG. 36). An increase in the concentration of IL-2 response was observed in the supernatants of CD4+T cells (FIG. 30, FIG. 31 and FIG. 32) as well as in the supernatants of CD8+T cells (FIG. 37, FIG. 38 and FIG. 39).

Biotinylated Anti PD-1 DNA Aptamer as Diagnostic Agent for PD-1 in Human Tissues with Immunohistochemistry

The PD-1 aptamer was examined on human tonsil and lung cancer tissues using IHC-DAB method. This determines the best working condition and dilution of this PD-1 apatmer. The best PD-1 aptamer positive staining was found at 1:1000 nM concentration with IHC-Tek Epitope Retrieval steam pretreatment (FIGS. 15B, 15C). No or weak positive staining was observed on proteinase K pretreatment and intact slides.

Target Cell Lysis Activity of Anti PD-1 DNA Aptamer Using Incucyte Annexin V-Based Assay

The functionality of anti PD-1 DNA aptamer was also validated using target cell lysis analysis by Incucyte. HEL.92.1.7 cells were used as target cells whereas PBMCs with T cell subpopulation were used as effector cells. Co-culturing of these two cells (effector+target cells) in the presence of Annexin V results in the mean total green object area. In the presence of PD-1 aptamer, an increase is seen in green object area comparable to presence of PD-1 antibody and a decrease is seen in green object area in control conditions. These results show that anti PD-1 DNA aptamer promotes T cell activation in lower concentration than the antibody (FIG. 41).

PEGlyated Anti PD-1 DNA Aptamer (Anti-PD-1-p10) Exhibits Improved Pharmacokinetics

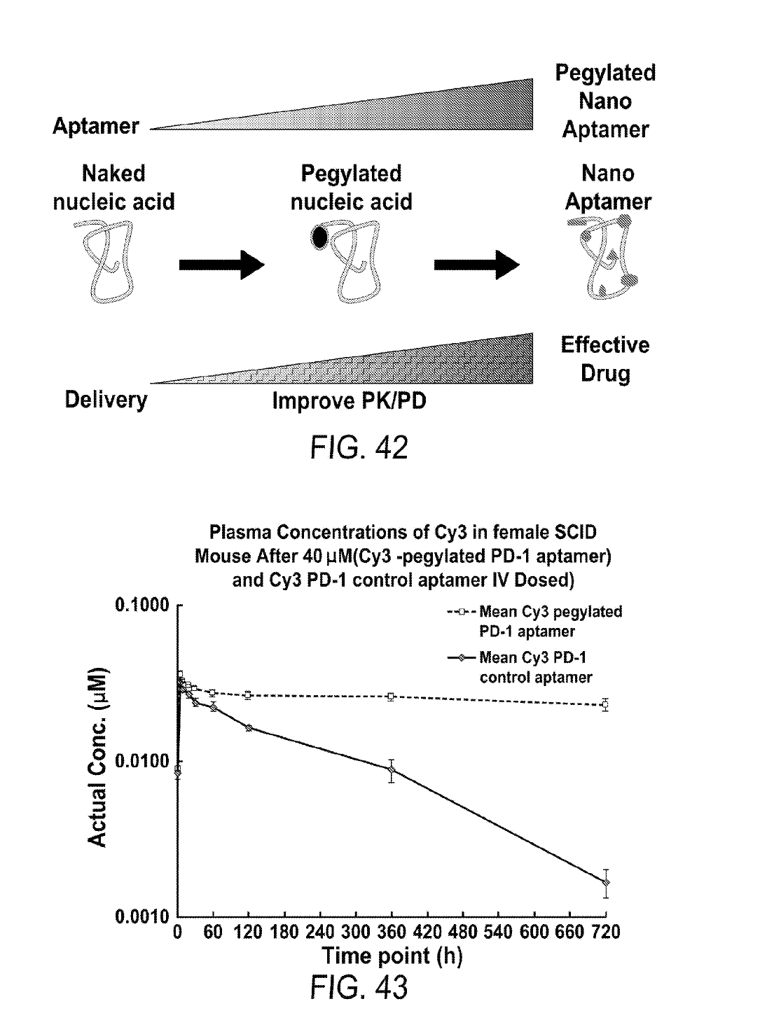

Conjugation of polyethylene glycol (PEG) to the biomolecules like aptamers (nucleic acids), proteins and vaccines is one of the most common and important strategy for improving the half-life and stability of these molecules in the blood and also to protect these molecules from recognition of immune cells as well as renal and hepatic clearance (Yoshihiro Morita et al Molecular Therapy-Nucleic acids (2016) 5, e399) (FIG. 42). Aptamer pharmacokinetics was determined in NOD/SCID female mice after a single intravenous dose of Cy3-labeled PD-1 and PD-1-p10. Compared with PD-1, conjugation of 10 kDa of PEG to anti PD-1 DNA aptamer reduced the clearance from the systemic circulation and extended the elimination half-life (FIG. 8A and FIG. 8B). This led to higher plasma concentrations of anti-PD-1-p10 for a prolonged period (117/360 min vs just cy3 37/360 min) and an at least 3-fold increase in overall systemic exposure (FIG. 43). The total volume of distribution volume steady state (Vs) was comparable between anti PD-1-p10 and anti PD-1.

Anti PD1 Aptamer-p10 Efficacy is Comparable to Anti-PD-1 Antibody

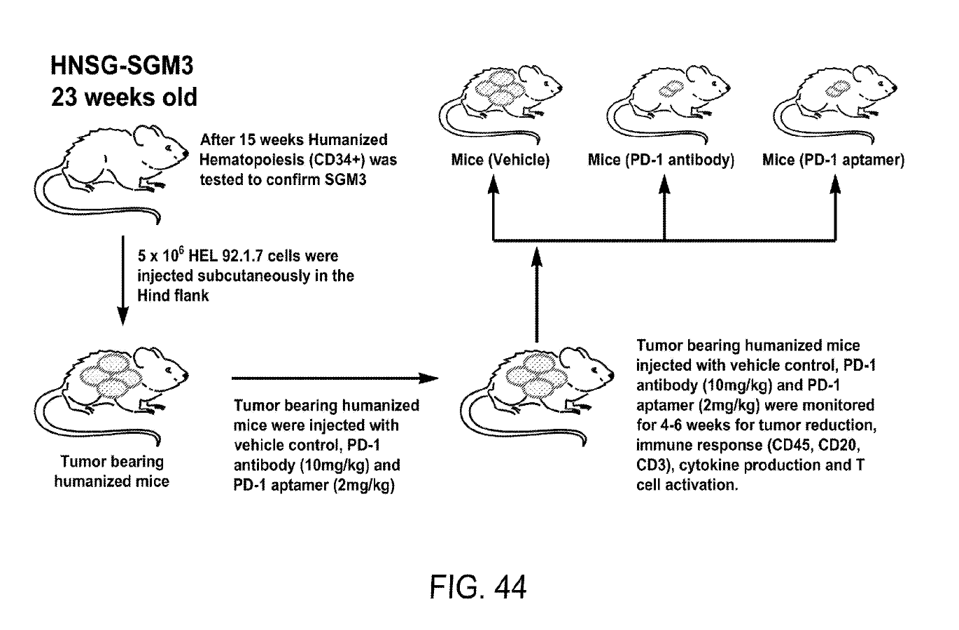

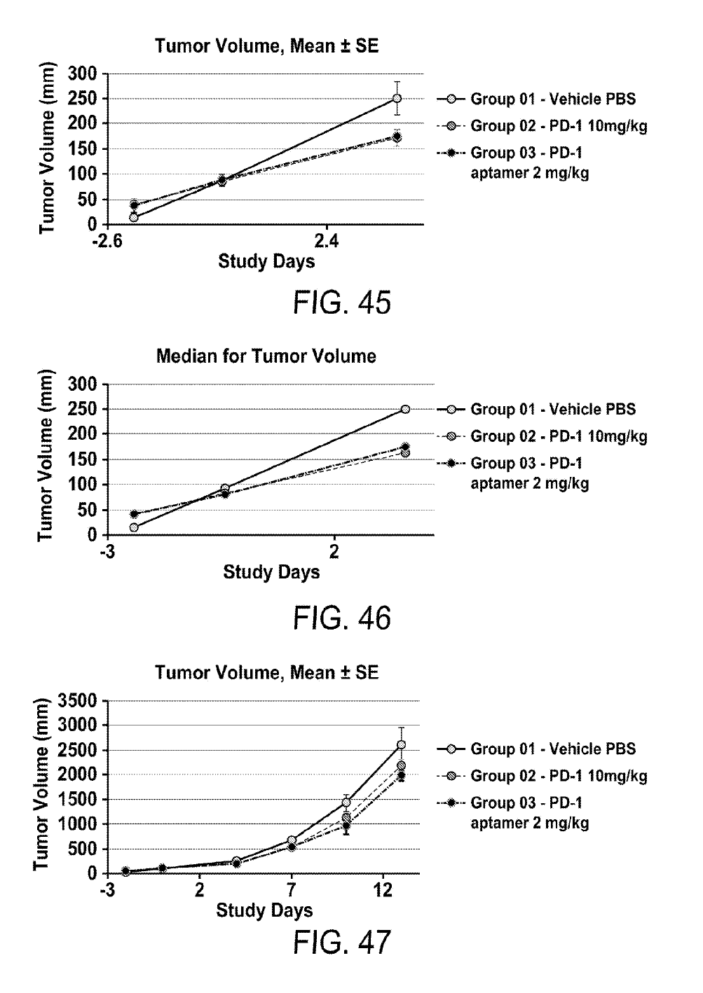

Anti PD1 aptamer efficacy was examined for comparison to the anti PD-1 antibody using HEL92.1.7 model to engraft in humanized NSG (FIG. 44). Tumor volume was observed at 3002.18 mm³ for control group vs. PD-1 Ab (2224.07 mm³) vs. PD-1 aptamer (2190.85 mm³) (FIGS. 45-47). This data shows that Anti PD1 aptamer-p10 reduces tumor burden comparable to Anti-PD-1 antibody in mice engrafted with hematological leukemic cells via the functional blockade of PD-1 and enhancement of immune response to clear tumor cells.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

1. A single-stranded DNA aptamer, comprising the formula
n-L-n-Z-n-R-n,
wherein “L” comprises the nucleic acid sequence TACCTGATAGCGTATGCGA (SEQ ID NO:2), or a nucleic acid sequence having at least 80% sequence identity to SEQ ID NO:2, or a fragment thereof at least 15 nucleotides in length,
wherein “Z” comprises the nucleic acid sequence GGGxTTGGTGTGGTGGG (SEQ ID NO:1), wherein “x” is A, T, or C,
wherein “R” comprises the nucleic acid sequence CTCTCAGTAGGTGCATAAGCG (SEQ ID NO:3), or a nucleic acid sequence having at least 80% sequence identity to SEQ ID NO:3, or a fragment thereof at least 15 nucleotides in length, and
wherein each “n” is independently any 0 to 10 nucleotides,
wherein the DNA aptamer specifically binds to PD-1 and disrupts the interaction between Programmed Death-1 (PD-1) and Programmed Death Ligand-1 (PD-L1) under physiological conditions.

2. The DNA aptamer of claim 1, wherein the DNA aptamer comprises a nucleic acid sequence selected from the group consisting of SEQ ID NO:6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, and 63.

3. The DNA aptamer of claim 1, wherein the DNA aptamer comprises a nucleic acid sequence having at least 80% sequence identity to a nucleic acid sequence selected from the group consisting of SEQ ID NO:6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, and 63.

4. A composition comprising the DNA aptamer of claim 1 in a pharmaceutically acceptable carrier.

5. The composition of claim 4, wherein the DNA aptamer is encapsulated in a nanoparticle.

6. The composition of claim 4, wherein the DNA aptamer is PEGylated.

7. The composition of claim 4, wherein the DNA aptamer is present at a concentration from 10 nM to 10 μM.

8. The composition of claim 4, further comprising an additional DNA aptamer.

9. The composition of claim 4, further comprising a DNA aptamer that specifically binds to CD38.

10. The composition of claim 4, further comprising a DNA aptamer that specifically binds to CD117.

11. The composition of claim 4, further comprising an antibody.

12. A method for treating cancer in a subject, comprising administering to the subject an effective amount of the composition of claim 4.

13. The method of claim 12, wherein the cancer is selected from the group consisting of melanoma, non-small-cell lung cancer (NSCLC), renal cell carcinoma (RCC), and bladder cancer.

14. The method of claim 12, wherein the cancer is a leukemia.

15. The method of claim 14, wherein the cancer is acute myeloid leukemia.

16. The method of claim 12, wherein the subject is a human.

17. A method for inhibiting the interaction between PD-1 and PD-L1 in a subject, comprising administering to the subject an effective amount of the composition of claim 4, wherein the DNA aptamer specifically binds to PD-1 and disrupts the interaction between Programmed Death-1 (PD-1) and Programmed Death Ligand-1 (PD-L1) under physiological conditions.

18. The method of claim 17, wherein the subject is suffering from a cancer selected from the group consisting of melanoma, non-small-cell lung cancer (NSCLC), renal cell carcinoma (RCC), and bladder cancer.

19. The method of claim 17, wherein the subject is suffering from acute myeloid leukemia.

20. The method of claim 17, wherein the subject is a human.


SEQUENCE LISTING (63 sequences)

SEQ ID NO: 1 - DNA, 17 bp, Artificial Sequence
  Synthetic construct
  misc_feature (4)..(4): n is A T or C
gggnttggtgtggtggg

SEQ ID NO: 2 - DNA, 19 bp, Artificial Sequence
  Synthetic construct
tacctgatagcgtatgcga

SEQ ID NO: 3 - DNA, 21 bp, Artificial Sequence
  Synthetic construct
ctctcagtaggtgcataagcg

SEQ ID NO: 4 - DNA, 65 bp, Artificial Sequence
  Synthetic construct
  misc_feature (20)..(44): N is any nucleotide
tacctgatagcgtatgcgannnnnnnnnnnnnnnnnnnnnnnnnctctcagtaggtgcataagcg

SEQ ID NO: 5 - DNA, 21 bp, Artificial Sequence
  Synthetic construct
cgcttatgcacctactgagag

SEQ ID NO: 6 - DNA, 83 bp, Artificial Sequence
  Synthetic construct
ccgtatacatacctgatagcgtatgcgagggtttggtgtggtggggctcttacctctcagtaggtgcataagcgcatgcatgc

SEQ ID NO: 7 - DNA, 83 bp, Artificial Sequence
  Synthetic construct
ccgtatacatacctgatagcgtatgcgagagggtttggtgtggtggggctctcctctcagtaggtgcataagcgcatgcatgc

SEQ ID NO: 8 - DNA, 83 bp, Artificial Sequence
  Synthetic construct
ccgtatacatacctgatagcgtatgcgagggtttggtgtggtggggctcttacctctcagtaggtgcataagcggcatgcata

SEQ ID NO: 9 - DNA, 83 bp, Artificial Sequence
  Synthetic construct
ccgtatacatacctgatagcgtatgcgagggattggtgtggtggggctcgcggctctcagtaggtgcataagcgcatgcatgc

SEQ ID NO: 10 - DNA, 83 bp, Artificial Sequence
  Synthetic construct
ccgtatacatacctgatagcgtatgcgagggtttggtgtggtggggctcggtactctcagtaggtgcataagcgcatgcatgc

SEQ ID NO: 11 - DNA, 83 bp, Artificial Sequence
  Synthetic construct
ccgtatacatacctgatagcgtatgcgaggaagggcttggtgtggtggggcttctctcagtaggtgcataagcgcatgcatgc

SEQ ID NO: 12 - DNA, 83 bp, Artificial Sequence
  Synthetic construct
ccgtatacatacctgatagcgtatgcgaggagagggattggtgtggtggggctctctcagtaggtgcataagcgcatgcatgc

SEQ ID NO: 13 - DNA, 83 bp, Artificial Sequence
  Synthetic construct
ccgtatacatacctgatagcgtatgcgaggaagggcttggtgtggtggggcttctctcagtaggtgcataagcggcatgcata

SEQ ID NO: 14 - DNA, 83 bp, Artificial Sequence
  Synthetic construct
ccgtatacatacctgatagcgtatgcgaccaagggcttggtgtggtggggcttctctcagtaggtgcataagcgcatgcatgc

SEQ ID NO: 15 - DNA, 83 bp, Artificial Sequence
  Synthetic construct
ccgtatacatacctgatagcgtatgcgaccaagggcttggtgtggtggggcttctctcagtaggtgcataagcggcatgcata

SEQ ID NO: 16 - DNA, 84 bp, Artificial Sequence
  Synthetic construct
ccgtatacatacctgatagcgtatgcgaggaagggcttggtgtggtgggggcttctctcagtaggtgcataagcgcatgcatgc

SEQ ID NO: 17 - DNA, 124 bp, Artificial Sequence
  Synthetic construct
ccgtatacatacctgatagcgtatgcgatataggagggtttggtgtggtgggctctcagtaggtgcataagcgttttgggatcctgggttcgtcctctcagtaggtgcataagcgcatgcatgc

SEQ ID NO: 18 - DNA, 84 bp, Artificial Sequence
  Synthetic construct
ccgtatacatacctgatagcgtatgcgaggaagtgggcttggtgtggtgggcttctctcagtaggtgcataagcgcatgcatgc

SEQ ID NO: 19 - DNA, 84 bp, Artificial Sequence
  Synthetic construct
ccgtatacatacctgatagcgtatgcgaggaagtgggcttggtgtggtgggcttctctcagtaggtgcataagcggcatgcata

SEQ ID NO: 20 - DNA, 83 bp, Artificial Sequence; identical to SEQ ID NO: 12
  Synthetic construct

SEQ ID NO: 21 - DNA, 83 bp, Artificial Sequence; identical to SEQ ID NO: 14
  Synthetic construct

SEQ ID NO: 22 - DNA, 128 bp, Artificial Sequence
  Synthetic construct
ccgtatacatacctgatagcgtatgcgatataggagggtttggtgtggtggggctctcagtaggtgcataagcgttttgggatcctgggttcgtcctctcagtaggtgcataagcgcatgcatgcatc

SEQ ID NO: 23 - DNA, 83 bp, Artificial Sequence; identical to SEQ ID NO: 6
  Synthetic construct

SEQ ID NO: 24 - DNA, 83 bp, Artificial Sequence; identical to SEQ ID NO: 8
  Synthetic construct

SEQ ID NO: 25 - DNA, 83 bp, Artificial Sequence; identical to SEQ ID NO: 7
  Synthetic construct

SEQ ID NO: 26 - DNA, 83 bp, Artificial Sequence; identical to SEQ ID NO: 10
  Synthetic construct

SEQ ID NO: 27 - DNA, 83 bp, Artificial Sequence
  Synthetic construct
ggcatatgttacctgatagcgtatgcgaggagggtttggtgtggtggggctcgctctcagtaggtgcataagcgcatgcatgc

SEQ ID NO: 28 - DNA, 83 bp, Artificial Sequence
  Synthetic construct
ggcatatgttacctgatagcgtatgcgaggagggtttggtgtggtggggctcgctctcagtaggtgcataagcggcatgcata

SEQ ID NO: 29 - DNA, 83 bp, Artificial Sequence
  Synthetic construct
ggcatatgttacctgatagcgtatgcgaggaagggcttggtgtggtggggcttctctcagtaggtgcataagcgcatgcatgc

SEQ ID NO: 30 - DNA, 83 bp, Artificial Sequence
  Synthetic construct
ggcatatgttacctgatagcgtatgcgaggagagggattggtgtggtggggctctctcagtaggtgcataagcgcatgcatgc

SEQ ID NO: 31 - DNA, 83 bp, Artificial Sequence
  Synthetic construct
ccgtatacatacctgatagcgtatgcgagagggtttggtgtggtggggctctcctctcagtaggtgcataagcggcatgcata

SEQ ID NO: 32 - DNA, 83 bp, Artificial Sequence
  Synthetic construct
ccgtatacatacctgatagcgtatgcgagggtttggtgtggtggggctcggtactctcagtaggtgcataagcggcatgcata

SEQ ID NO: 33 - DNA, 83 bp, Artificial Sequence
  Synthetic construct
ccgtatacatacctgatagcgtatgcgatataggagggtttggtgtggtggggctctcagtaggtgcataagcgcatgcatgc

SEQ ID NO: 34 - DNA, 83 bp, Artificial Sequence
  Synthetic construct
ccgtatacatacctgatagcgtatgcgaggtgggagggattggtgtggtggggctctcagtaggtgcataagcgcatgcatgc

SEQ ID NO: 35 - DNA, 83 bp, Artificial Sequence
  Synthetic construct
ccgtatacatacctgatagcgtatgcgacctaggagggtttggtgtggtggggctctcagtaggtgcataagcgcatgcatgc

SEQ ID NO: 36 - DNA, 83 bp, Artificial Sequence
  Synthetic construct
ccgtatacatacctgatagcgtatgcgacctaggagggtttggtgtggtggggctctcagtaggtgcataagcggcatgcata

SEQ ID NO: 37 - DNA, 83 bp, Artificial Sequence
  Synthetic construct
ccgtatacatacctgatagcgtatgcgatataggagggtttggtgtggtggggctctcagtaggtgcataagcggcatgcata

SEQ ID NO: 38 - DNA, 83 bp, Artificial Sequence
  Synthetic construct
ccgtatacatacctgatagcgtatgcgaggtgggagggattggtgtggtggggctctcagtaggtgcataagcggcatgcata

SEQ ID NO: 39 - DNA, 83 bp, Artificial Sequence
  Synthetic construct
ccgtatacatacctgatagcgtatgcgaggagagggattggtgtggtggggctctctcagtaggtgcataagcggcatgcata

SEQ ID NO: 40 - DNA, 83 bp, Artificial Sequence; identical to SEQ ID NO: 39
  Synthetic construct

SEQ ID NO: 41 - DNA, 83 bp, Artificial Sequence; identical to SEQ ID NO: 15
  Synthetic construct

SEQ ID NO: 42 - DNA, 126 bp, Artificial Sequence
  Synthetic construct
ccgtatacatacctgatagcgtatgcgaggaagtgggcttggtgtggtgggcttctctcagtaggtgcataagcgttttgggatcctgggttcgtcctctcagtaggtgcataagcgcatgcatgc

SEQ ID NO: 43 - DNA, 83 bp, Artificial Sequence; identical to SEQ ID NO: 31
  Synthetic construct

SEQ ID NO: 44 - DNA, 83 bp, Artificial Sequence; identical to SEQ ID NO: 32
  Synthetic construct

SEQ ID NO: 45 - DNA, 128 bp, Artificial Sequence
  Synthetic construct
ccgtatacatacctgatagcgtatgcgaggaagggcttggtgtggtggggcttctctcagtaggtgcataagcgttttgggatcctgggttcgtcctctcagtaggtgcataagcgcatgcatgcatc

SEQ ID NO: 46 - DNA, 128 bp, Artificial Sequence
  Synthetic construct
ccgtatacatacctgatagcgtatgcgaggagagggattggtgtggtggggctctctcagtaggtgcataagcgttttgggatcctgggttcgtcctctcagtaggtgcataagcgcatgcatgcatc

SEQ ID NO: 47 - DNA, 128 bp, Artificial Sequence
  Synthetic construct
ccgtatacatacctgatagcgtatgcgagagggtttggtgtggtggggctctcctctcagtaggtgcataagcgttttgggatcctgggttcgtcctctcagtaggtgcataagcgcatgcatgcatc

SEQ ID NO: 48 - DNA, 125 bp, Artificial Sequence
  Synthetic construct
ccgtatacatacctgatagcgtatgcgagggtttggtgtggtggggctcttacctctcagtaggtgcataagcgttttgggatcctgggttcgtcctctcagtaggtgcataagcgcatgcatgc

SEQ ID NO: 49 - DNA, 125 bp, Artificial Sequence; identical to SEQ ID NO: 48
  Synthetic construct

SEQ ID NO: 50 - DNA, 125 bp, Artificial Sequence
  Synthetic construct
ccgtatacatacctgatagcgtatgcgagagggtttggtgtggtggggctctcctctcagtaggtgcataagcgttttgggatcctgggttcgtcctctcagtaggtgcataagcgcatgcatgc

SEQ ID NO: 51 - DNA, 83 bp, Artificial Sequence
  Synthetic construct
ccgtatacatacctgatagcgtatgcgagggattggtgtggtggggctcgcggctctcagtaggtgcataagcggcatgcata

SEQ ID NO: 52 - DNA, 134 bp, Artificial Sequence
  Synthetic construct
ccgtatacatacctgatagcgtatgcgaggtgggagggattggtgtggtggggctctcagtaggtgcataagcggagttcgttcttctgttgtgtttctctctctcagtaggtgcataagcgcatgcatgcatc

SEQ ID NO: 53 - DNA, 82 bp, Artificial Sequence
  Synthetic construct
ccgtatacatacctgatagcgtatgcgatataggagggtttggtgtggtgggctctcagtaggtgcataagcggcatgcata

SEQ ID NO: 54 - DNA, 82 bp, Artificial Sequence
  Synthetic construct
ccgtatacatacctgatagcgtatgcgatataggagggtttggtgtggtgggctctcagtaggtgcataagcgcatgcatgc

SEQ ID NO: 55 - DNA, 83 bp, Artificial Sequence; identical to SEQ ID NO: 36
  Synthetic construct

SEQ ID NO: 56 - DNA, 83 bp, Artificial Sequence
  Synthetic construct
ggcatatgttacctgatagcgtatgcgaatcgtgagggtttggtgtggtggggctctcagtaggtgcataagcgcatgcatgc

SEQ ID NO: 57 - DNA, 83 bp, Artificial Sequence
  Synthetic construct
ggcatatgttacctgatagcgtatgcgaatcgtgagggtttggtgtggtggggctctcagtaggtgcataagcggcatgcata

SEQ ID NO: 58 - DNA, 127 bp, Artificial Sequence
  Synthetic construct
ccgtatacatacctgatagcgtatgcgaggagagggattggtgtggtggggctctctcagtaggtgcataagcgatagcgtatgcgatttgtttcgttcggatctgtatattctctcagtaggtgca

SEQ ID NO: 59 - DNA, 130 bp, Artificial Sequence
  Synthetic construct
ccgtatacatacctgatagcgtatgcgaggtgggagggattggtgtggtgggctctcagtaggtgcataagcggagttcgttcttctgttgtgtttctctctctcagtaggtgcataagcgcatgcatgc

SEQ ID NO: 60 - DNA, 110 bp, Artificial Sequence
  Synthetic construct
ccgtatacatacctgatagcgtatgcgatgaccctcgttcgtctctctctcccctctcagtcgttggtagtctcttttgtctctcagtaggtgcataagcgcatgcatgc

SEQ ID NO: 61 - DNA, 83 bp, Artificial Sequence; identical to SEQ ID NO: 35
  Synthetic construct

SEQ ID NO: 62 - DNA, 111 bp, Artificial Sequence
  Synthetic construct
ccgtatacatacctgatagcgtatgcgatgaccctcgttcgtctctctctcccctctcagtcgttggtagtctcttttgtctctcagtaggtgcataagcgcatgcatgcc

SEQ ID NO: 63 - DNA, 118 bp, Artificial Sequence
  Synthetic construct
gcgaggagagggattggtgtggtggggctctctcagtaggtgcataagcgatagcgtatgcgatttgtttcgttcggatctgtatattctctcagtaggtgcataagcgcatgcatgc